(19) United States
(12) Patent Application Publication
(10) Pub. No.: US 2015/0164835 A1
(43) Pub. Date: Jun. 18, 2015

(54) Opioid Salts with Release Properties and Characteristics Useful for Abuse Deterrent Drug Product Formulations

(71) Applicant: Pisgah Laboratories, Inc., Pisgah Forest, NC (US)

(72) Inventors: Clifford Riley King, Pisgah Forest, NC (US); Stephen G. D'Ambrosio, Pisgah Forest, NC (US); Michael L. English, Pisgah Forest, NC (US); David W. Bristol, Pisgah Forest, NC (US)

(73) Assignee: Pisgah Laboratories, Inc.

(21) Appl. No.: 14/628,344

(22) Filed: Feb. 23, 2015

Related U.S. Application Data
(63) Continuation-in-part of application No. 13/723,370, filed on Dec. 21, 2012.
Application No. 13/723,370 is a continuation-in-part of application No. 12/080,513, filed on Apr. 3, 2008, now Pat. No. 8,859,622.
Application No. 12/080,513 is a continuation-in-part of application No. 12/080,514, filed on Apr. 3, 2008, now Pat. No. 8,575,151.
Application No. 12/080,514 is a continuation-in-part of application No. 12/080,531, filed on Apr. 3, 2008, now abandoned.
Application No. 12/080,531 is a continuation-in-part of application No. 13/334,842, filed on Dec. 22, 2011, now Pat. No. 8,334,322.
Application No. 13/334,842 is a continuation-in-part of application No. 11/805,225, filed on May 22, 2007, now abandoned.
Application No. 13/723,370 is a continuation-in-part of application No. 13/313,870, filed on Dec. 7, 2011.
Application No. 13/313,870 is a division of application No. 11/973,252, filed on Oct. 5, 2007.
Application No. 13/723,370 is a continuation-in-part of application No. 12/537,664, filed on Aug. 7, 2009.
Application No. 12/537,664 is a division of application No. 11/973,252, filed on Oct. 5, 2007.

(51) Int. Cl. A61K 31/194; A61K 31/192; A61K 45/06; A61K 9/20; A61K 9/48; A61K 9/00; A61K 31/485; A61K 31/4748

(52) U.S. Cl. CPC A61K 31/194; A61K 31/485; A61K 31/192; A61K 31/4748; A61K 9/20; A61K 9/48; A61K 9/0019; A61K 45/06

(57) ABSTRACT

A drug substance, and drug products comprising the drug substance, wherein the drug substance is selected from the group consisting of amorphous oxymorphone pamoate; polymorphic oxymorphone pamoate; oxymorphone xinafoate; amorphous codeine pamoate; codeine xinafoate; amorphous levorphanol pamoate; polymorphic levorphanol pamoate; levorphanol xinafoate; amorphous naltrexone pamoate; polymorphic naltrexone pamoate and naltrexone xinafoate.

### CROSS-REFERENCE TO RELATED APPLICATIONS

This application is a continuation-in-part of pending U.S. patent application Ser. No. 13/723,370 filed Dec. 21, 2012 which in turn is a continuation-in-part of each of U.S. patent application Ser. No. 12/080,513 filed Apr. 3, 2008 now U.S. Pat. No. 8,859,622 issued Oct. 14, 2014; U.S. patent application Ser. No. 12/080,514 filed Apr. 3, 2008 now U.S. Pat. No. 8,575,151 issued Nov. 5, 2013; U.S. patent application Ser. No. 12/080,531 filed Apr. 3, 2008 which is abandoned and U.S. patent application Ser. No. 13/334,842 filed Dec. 22, 2011 now U.S. Pat. No. 8,334,322 issued Dec. 18, 2012 each of which is incorporated herein by reference. Each of U.S. patent application Ser. No. 12/080,513; U.S. patent application Ser. No. 12/080,514; U.S. patent application Ser. No. 12/080,531 and U.S. patent application Ser. No. 13/334,842 is, in turn, a divisional application of abandoned U.S. patent application Ser. No. 11/805,225 filed May 22, 2007 which is incorporated herein by reference. U.S. patent application Ser. No. 13/723,370 filed Dec. 21, 2012 is also a continuation-in-part of pending U.S. patent application Ser. No. 13/313,870 filed Dec. 7, 2011 which is, in-turn, a divisional application of pending U.S. patent application Ser. No. 11/973,252 filed Oct. 5, 2007 each of which is incorporated herein by reference. U.S. patent application Ser. No. 13/723,370 filed Dec. 21, 2012 is also a continuation-in-part of pending U.S. patent application Ser. No. 12/537,664 filed Aug. 7, 2009 which is, in-turn, a divisional application of pending U.S. patent application Ser. No. 11/973,252 filed Oct. 5, 2007 each of which is incorporated herein by reference.

### BACKGROUND

The present invention is related to salts of oxymorphone, codeine, levorphanol and naltrexone. More specifically, the present invention is related to organic acid addition salts of oxymorphone, codeine, levorphanol and naltrexone which are less resistant to abuse or dose dumping. Even more specifically, the present invention is directed to amorphous and polymorphic forms of oxymorphone pamoate, oxymorphone xinafoate, codeine pamoate, codeine xinafoate, levorphanol pamoate, levorphanol xinafoate, naltrexone pamoate and naltrexone xinafoate.

Abuse deterrent performance features found in abuse potential drug products are generally introduced by five methodologies that, in one form or another, interfere with intentional abuse by someone seeking to get “high”. These methodologies are: 1) drug product formulation with excipients that interfere with the extraction of the active ingredient, 2) formation of the drug product tablet to resist physical manipulation such as grinding or milling to facilitate isolation of the active ingredient, 3) introduction of one or more active ingredients which release upon product manipulation and interfere with an abuser getting “high”, 4) introduction of abuse deterrent features at the drug substance level, and 5) some combination of the previous four methodologies. Of course, one other method to reduce abuse of drug products is by administrative or bureaucratic means which are generally ineffective, costly, and provide an undue hardship and burden on the citizenry of the Nation that are not abusing the drug. This latter approach is not considered herein and the present invention focuses on expanding the existing knowledge base of abuse deterrent features introduced at the drug substance level. The inclusion of naltrexone relates to methodology “3” listed above. For explicit understanding of the invention herein, the introduction of abuse deterrent features at the drug substance level are certainly compatible with other lines of defense against abuse that are often obtained through formulation and manufacturing techniques.

Herein the term “drug substance” is used as in the art to define the pharmaceutical active amine with counterions as necessary to achieve charge neutrality without any other additional compound and is therefore specific to the pharmaceutically active amine as it would be formulated into a drug product wherein a drug product is a combination of a drug substance and additional components. The current, commercially available salts preferably used to prepare the compounds of interest herein are: oxymorphone hydrochloride, codeine sulfate, levorphanol tartrate and naltrexone hydrochloride. These compounds are all highly water soluble over all biologically relevant pH ranges and are also quite soluble in acidic ethanol media. Therefore, formulation and processing techniques are typically relied upon to provide dosage strengths and dissolution profiles other than immediate release, wherein release of the drug substance from the drug product is retarded. Despite the ongoing efforts, it is generally not a viable route to impede intentional drug abuse by a would-be abuser.

Pamoate and xinafoate salt families of amine-containing medicinally useful active ingredients have received relatively little attention and have experienced limited commercial success. Only one xinafoate salt, salmeterol xinafoate, is currently listed in the US Food and Drug Administration's Orange Book of drug products as having received market approval. Similarly, only four pamoate salts have current market approval: hydroxyzine, imipramine, olanzapine and triptorelin. Pyrvinium pamoate once had FDA market approval but has been discontinued.

Hydroxyzine pamoate in a drug product is available as a capsule or suspension for oral administration. Originally, hydroxyzine was provided as the dihydrochloride salt. The drug substance contained the rather lipophilic aryl chloride substituent and the compound's two basic nitrogen constituents, as hydrochloride salts, helped overcome insolubility of the active. However, one nitrogen contained a two ethylene oxide residue substituent with this hydroxyethyl ethyl ether providing substantial water solubility. With an enhanced water solubility profile, water soluble products were formulated as syrups and for injection. In fact, the Merck Index reports the dihydrochloride salt as having a solubility in water of <700 mg/mL whereas the pamoate salt is “practically insoluble in water”.

Additional information related to the historical development of pamoate salts may be found in U.S. Pat. No. 8,039,461 to Audia et al. entitled, “Physical States of a Pharmaceutical Drug Substance”, the disclosure of which is incorporated herein by reference in its entirety. U.S. Pat. No. 7,718,649 to King et al., also incorporated herein in its entirety, wherein described are various amorphous and polymorphic compositions of imipramine pamoate.

Amorphous materials of other, non-opioid, pamoate salts have been reported in the literature. In U.S. Pat. No. 4,076,942 to Smith et al. entitled “Crystalline Dipilocarpinium Pamoate”, describes the amorphous form of this compound as presenting “a drawback in not being readily and easily handleable, in being difficult to formulate in an appropriate ocular delivery system and in being difficult to generate stoichiometrically”. Solvated forms of the title compounds were prepared which, through a de-solvating process, yielded crystalline dipilocarpinium pamoate.

U.S. Pat. No. 8,846,766 to King et al. entitled, “Abuse-Deterrent Methadone for the Safe Treatment of Drug Abuse and Pain Relief”, the disclosure of which is incorporated herein in its entirety, addresses what could be considered a cross-over compound from being used for pain relief to a drug abuse treatment medication. Methadone is used extensively in the United States for the treatment of drug abuse, but unfortunately, it too is abused and often leads to death of the abuser. Safe forms of methadone, as the pamoate salt, are taught in the referenced patent.

History has shown that principally, abuse deterrent drug product features are dependent upon the difficulty of separating the drug substance from excipients used to formulate the dosage product. Solubilizing extraction techniques are generally employed. Further, the physical nature of dosage presentation, particularly the inability of the tablet to be finely ground can influence how well an extraction can occur. Since the readily available commercial drug substances are essentially completely water soluble, extraction of the drug substance from the formulated product is usually easy. Use of extraction interfering excipients, or the pressing of hard tablets merely frustrates the potential abuser, but no real barrier has been constructed against the would-be abuser.

Dose dumping is a popular method for abusers to obtain a quick release of the drug substance from the formulated product. In this case, the abuser swallows the drug product along with ethanol of their liking to effectively release the drug content immediately from the formulated drug product. If this activity is performed with extended release products or with multiple immediate release formulations, then it is highly likely death may ensue. Essentially all of the traditional drug substance salts of interest to an abuser will dose dump.

In conjunction with, or absent extraction techniques employed by the abuser, milling the drug product to a fine powder can be advantageous for getting high with drug products based on highly water soluble drug substances. Besides oral routes of abuse, application of finely ground drug product, or drug substance extracted from drug products, directly to the mucosal membranes is a superior way of achieving a “high”. The mucosal membranes include nasal, buccal, rectal, vaginal and optic interfaces to the blood stream. Drug substance solubility and pH dependent release is critical for these routes of administration to be effective for the drug abuser.

The United States Food and Drug Administration has categorized different levels of abuse deterrence for drug product in a Guidance for Industry document entitled, “Abuse Deterrent Opioids—Evaluation and Labeling”. In this document the FDA outlines four Tiers of abuse deterrence with respect to drug product labeling. Tier 1 covers claims that a product is formulated with physicochemical barriers to abuse. Tier 2 addresses claims that a product is expected to reduce or block the effects of the opioid when the product is manipulated. Tier 3 covers claims that a product is expected to result in a meaningful reduction in abuse, and Tier 4 label claims indicate that a product has demonstrated reduced abuse in the community.

In spite of the ongoing effort those of skill in the art still do not have an adequate way to mitigate abuse of controlled substances. Those of skill in the art especially seek a platform for mitigating abuse which is not dependent on the matrix since it is well established that abuse resistance, or dose dumping, based on the matrix is easily defeated by separating the drug substance from the matrix.

### SUMMARY OF THE INVENTION

It is an object of the present invention to provide organic acid addition salts of opioid compounds selected from the group consisting of but not limited to oxymorphone, codeine, levorphanol and naltrexone.

A particular feature of the present invention is that the organic acid addition salts of the opioid family of alkaloids are available in amorphous and polymorphic forms.

It is an object of the present invention to provide drug substances possessing abuse deterrent properties useful for formulation in abuse deterrent drug products.

It is an object of the present invention to provide drug substances which are essentially insoluble in human mucosal membranes particularly, human nasal, buccal, optic, vaginal or rectal membranes.

A particular feature of the present invention is that the amine-containing active pharmaceutical ingredients do not readily release form the drug substance in an aqueous solution within a pH window of about 4 to about 9 which interferes with the direct isolation of the active pharmaceutical (API) outside of the pH window.

A pharmaceutical composition is provided which is particularly advantageous with regards to patient safety. The composition comprises a salt of an opioid wherein the salt prohibits the opioid from being susceptible to abuse particularly with regards to dose dumping.

An embodiment of the invention is provided by a process for forming a drug substance wherein at least one equivalent of the amine containing drug substance is reacted per mole of disodium pamoate to yield the drug substance pamoic acid salt, preferably in a ratio of 2:1, 1:1, or mixtures thereof. An aqueous acidic solution of the amine containing drug substance is combined with a basic solution of pamoic acid or disodium pamoate. The acid/base reaction ensues and the insoluble organic acid salt precipitates from the aqueous solution. Optionally, the salt can be purified, dried and milled to obtain a drug substance ready for formulation into the desired delivery format. The drug product formulated with the drug substances then possesses the targeted delivery characteristics of the drug substance and the potential for abuse of either the drug substance and/or drug product is eliminated or greatly reduced when abuse is attempted via the mucosal surfaces or by injection.

It is an object of the present invention to provide oxymorphone pamoate, oxymorphone xinafoate, codeine pamoate, codeine xinafoate, levorphanol pamoate, levorphanol xinafoate, naltrexone pamoate, and naltrexone xinafoate drug substances which are not susceptible to dose dumping.

It is an object of the present invention to provide both amorphous and polymorphic oxymorphone pamoate and oxymorphone xinafoate suitable for use in abuse deterrent products.

It is an object of the present invention to provide amorphous codeine pamoate and codeine xinafoate suitable for use in abuse deterrent products.

It is an object of the present invention to provide both amorphous and polymorphic levorphanol pamoate and levorphanol xinafoate suitable for use in abuse deterrent products.

It is an object of the present invention to provide both amorphous and polymorphic naltrexone pamoate and naltrexone xinafoate suitable for use in abuse deterrent products.

It is an object of the present invention to provide a formulation compatible naltrexone derivative for use with organic acid addition salts of amine containing opioids wherein the naltrexone derivative is selected from the group consisting of amorphous naltrexone pamoate, polymorphic naltrexone pamoate and naltrexone xinafoate.

It is an object of the present invention to provide extended release drug products controlled by the release rates of the pamoate or xinafoate drug substance salt in 0.1N HCl where the release rate is less than eighty percent of the release rate of the corresponding mineral acid or tartrate salt form of the drug substance.

It is an object of the present invention to provide extended release drug products wherein the drug product is enterically coated and release occurs predominantly in the bowel.

It is an object of the present invention to provide an immediate release drug product comprising polymorphic oxymorphone pamoate as a drug substance.

It is an object of the present invention to provide drug products that do not dose dump defined as less than about ninety percent of the active being released from its salt form relative to the comparable mineral acid, or tartaric acid salt of the drug substance in the presence of alcohol.

It is an object of the present invention to provide organic acid addition salts of opioid compounds wherein the organic acid component is defined as Structure A further herein.

A particular feature of the present invention is that the organic acid addition salts of the opioid family of alkaloids are available in amorphous and polymorphic forms.

A feature of the present invention is robust and stable drug product formulations prepared from the organic acid addition salts of the opioid family of alkaloids.

It is yet another feature of the present invention to provide tamper resistant and/or tamper proof drug product formulations employing the organic acid addition salts of the opioid family of alkaloids.

It is another feature of the present invention to provide organic acid addition salts of the opioid family of alkaloids, when employed with an anti-abuse formulation technique, impart at least two abuse deterrent mechanisms into the drug product.

It is a feature of the present invention to employ physical and chemical means to prepare abuse deterrent controlled substance formulations.

In an embodiment of the present invention, the controlled drug substance is an amine-containing organic salt which releases from the organic salt slowly in the pH window of about 4 to about 9.

These and other advantages, as will be realized, are provided in a drug substance comprising a pharmaceutically acceptable organic acid addition salt of an active pharmaceutical selected from oxymorphone, codeine, levorphanol and naltrexone wherein said organic acid is selected from Structure A:

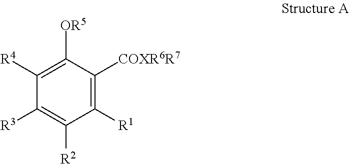

wherein R¹-R⁴ are independently selected from H, alkyl or substituted alkyl of 1-6 carbons, adjacent groups may be taken together to form a cyclic alkyl, cyclic alkyl-aryl, or cyclic aryl moiety;
R⁵ is selected from H, or an alkali earth cation;
R⁶ and R⁷ are independently selected from H, alkyl of 1-6 carbons, an alkali earth cation, and aryl of 6 to 12 carbons, in a number sufficient to complete the valence bonding of X, and
wherein X is selected from nitrogen, oxygen or sulfur; and
wherein the drug substance has a morphology selected from amorphous and crystalline.

Yet another embodiment is provided in a drug product comprising a drug substance comprising a pharmaceutically acceptable organic acid addition salt of an active pharmaceutical selected from oxymorphone, codeine, levorphanol and naltrexone wherein the organic acid is selected from Structure A:

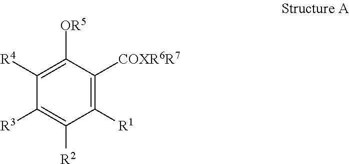

wherein R¹-R⁴ are independently selected from H, alkyl or substituted alkyl of 1-6 carbons, adjacent groups may be taken together to form a cyclic alkyl, cyclic alkyl-aryl, or cyclic aryl moiety;
R⁵ is selected from H, or an alkali earth cation;
R⁶ and R⁷ are independently selected from H, alkyl of 1-6 carbons, an alkali earth cation, and aryl of 6 to 12 carbons, in a number sufficient to complete the valence bonding of X, and
wherein X is selected from nitrogen, oxygen or sulfur and
wherein less than 85 wt % of said opioid is released at a biological pH in 1 hour.

Yet another embodiment is provided in a method of administering an active pharmaceutical comprising providing an opioid containing pharmaceutically active compound in a dose suitable for achieving a therapeutic dose of said opioid in a predetermined time wherein said therapeutic dose is not exceeded by ingestion of alcohol at biological pH.

These and other advantages, as will be realized, are provided in a drug substance consisting essentially of a pharmaceutically acceptable organic acid addition salt of an amine containing pharmaceutically active compound wherein said drug substance is selected from the group consisting of:

amorphous oxymorphone pamoate characterized by at least one method selected from the group consisting of:
a differential scanning calorimeter thermogram of FIG. 1;

### an FTIR of FIG. 2;

an X-ray diffraction diffractogram of FIG. 3; and
a ¹H NMR spectrum of FIG. 4;
polymorphic oxymorphone pamoate characterized by at least one method selected from the group consisting of:
a differential scanning calorimeter thermogram of FIG. 5;

### an FTIR of FIG. 6;

an X-ray diffraction diffractogram of FIG. 7; and
a ¹H NMR spectrum of FIG. 8;
oxymorphone xinafoate characterized by at least one method selected from the group consisting of:
a differential scanning calorimeter thermogram of FIG. 9

### an FTIR of FIG. 10;

an X-ray diffraction diffractogram of FIG. 11; and
a ¹H NMR spectrum of FIG. 12;
amorphous codeine pamoate characterized by at least one method selected from the group consisting of:
a differential scanning calorimeter thermogram of FIG. 13;

### an FTIR of FIG. 14;

an X-ray diffraction diffractogram of FIG. 15; and
a ¹H NMR spectrum of FIG. 16;
codeine xinafoate characterized by at least one method selected from the group consisting of:
a differential scanning calorimeter thermogram of FIG. 17;

### an FTIR of FIG. 18;

an X-ray diffraction diffractogram of FIG. 19; and
a ¹H NMR spectrum of FIG. 20;
amorphous levorphanol pamoate characterized by at least one method selected from the group consisting of:
a differential scanning calorimeter thermogram of FIG. 21;

### an FTIR of FIG. 22;

an X-ray diffraction diffractogram of FIG. 23; and
a ¹H NMR spectrum of FIG. 24;
polymorphic levorphanol pamoate characterized by at least one method selected from the group consisting of:
a differential scanning calorimeter thermogram of FIG. 25;

### an FTIR of FIG. 26;

an X-ray diffraction diffractogram of FIG. 27; and
a ¹H NMR spectrum of FIG. 28;
levorphanol xinafoate characterized by at least one method selected from the group consisting of:
a differential scanning calorimeter thermogram of FIG. 29;

### an FTIR of FIG. 30;

an X-ray diffraction diffractogram of FIG. 31; and
a ¹H NMR spectrum of FIG. 32;
amorphous naltrexone pamoate characterized by at least one method selected from the group consisting of:
a differential scanning calorimeter thermogram of FIG. 33;

### an FTIR of FIG. 34;

an X-ray diffraction diffractogram of FIG. 35; and
a ¹H NMR spectrum of FIG. 36;
polymorphic naltrexone pamoate characterized by at least one method selected from the group consisting of:
a differential scanning calorimeter thermogram of FIG. 37;

### an FTIR of FIG. 38;

an X-ray diffraction diffractogram of FIG. 39; and
a ¹H NMR spectrum of FIG. 40; and
naltrexone xinafoate characterized by at least one method selected from the group consisting of:
a differential scanning calorimeter thermogram of FIG. 41;

### an FTIR of FIG. 42;

an X-ray diffraction diffractogram of FIG. 43; and
a ¹H NMR spectrum of FIG. 44.

Yet another embodiment is provided in an oral dose drug product exhibiting preferential release in the bowel wherein the drug product comprises a drug substance selected from the group consisting of oxymorphone xinafoate and codeine xinafoate.

Yet another embodiment is provided in a solid oral dose drug product comprising at least one drug substance selected from the group consisting of: amorphous oxymorphone pamoate characterized by:

an extended release of said oxymorphone from said pamoate in 0.1 N HCl; or
a rate of release of said oxymorphone from said pamoate in 0.1 N HCl which is not exceeded by a rate of release of said oxymorphone from said pamoate in 0.1 N HCl with 5% ethanol;
polymorphic oxymorphone pamoate characterized by at one of:
immediate release of said oxymorphone from said pamoate in 0.1 N HCl; or
extended release of said oxymorphone from said pamoate at pH 4.5;
oxymorphone xinafoate characterized by at least one of:
extended release of said oxymorphone from said xinafoate in water; or
immediate release of said oxymorphone from said xinafoate at pH 6.8;
amorphous codeine pamoate characterized by at least one of:
extended releases of said codeine from said pamoate in 0.1 N HCl; or
a rate of release of said codeine from said pamoate in 0.1 N HCl which is not exceeded by a rate of release of said codeine from said pamoate in 0.1 N HCl with 5% ethanol; codeine xinafoate characterized by at least one of:
extended release of said codeine from said xinafoate in water; or
immediate release of said codeine from said xinafoate at pH 6.8;
amorphous levorphanol pamoate characterized by at least one of:
extended release of said levorphanol from said pamoate in 0.1 N HCl; or
extended release of said levorphanol from said pamoate in 0.1 N HCl with 5% ethanol;
polymorphic levorphanol pamoate characterized by at least one of:
extended release of said levorphanol from said pamoate in 0.1 N HCl; or
a rate of release of said levorphanol from said pamoate in 0.1 N HCl which is not exceeded by a rate of release of said levorphanol from said pamoate in 0.1 N HCl with 20% ethanol;
levorphanol xinafoate characterized by at least one of:
extended release of said levorphanol from said xinafoate in 0.1 N HCl; or
a rate of release of said levorphanol from said xinafoate in 0.1 N HCl which is not exceeded by a rate of release of said levorphanol from said xinafoate in 0.1 N HCl with 5% ethanol;
amorphous naltrexone pamoate characterized by at least one of:
extended release of said naltrexone from said pamoate in 0.1 N HCl; or
extended release of said naltrexone from said pamoate in 0.1 N HCl with ethanol;
polymorphic naltrexone pamoate characterized by at least one of:
extended release of said naltrexone from said pamoate in 0.1 N HCl; or
   extended release of said naltrexone from said pamoate at pH 4.5; and
   naltrexone xinafoate characterized by at least one of:
   extended release of said naltrexone from said xinafoate in 0.1 N HCl; or
   a rate of release of said naltrexone from said xinafoate in 0.1 N HCl which is not exceeded by a rate of release of said naltrexone from said xinafoate in 0.1 N HCl with 5% ethanol.

Yet another embodiment is provided in a drug product comprising: a drug substance consisting of an organic acid addition salt of naltrexone wherein said organic acid addition salt is defined by Structure A:

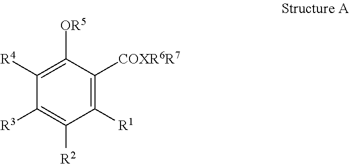

wherein:
R¹-R⁴ are independently selected from H, alkyl or substituted alkyl of 1-6 carbons, adjacent groups may be taken together to form a cyclic alkyl or cyclic aryl moiety;
R⁵ represents H, alkyl, alkylacyl or arylacyl;
R⁶ and R⁷ are independently selected from H, alkyl of 1-6 carbons, aryl of 6-12 carbons, alkylacyl or arylacyl analogues sufficient to satisfy the valence of X;
X is selected from nitrogen, oxygen or sulfur, and when X═O, R⁶+R⁷ may represent an alkali earth cation, ammonium or together form a heterocyclic moiety.

Yet another embodiment is provided in a method of administering an active pharmaceutical comprising: providing a drug substance selected from the group consisting of amorphous oxymorphone pamoate, amorphous codeine pamoate, polymorphic levorphanol pamoate and levorphanol xinafoate; forming a drug product comprising said drug substance suitable for achieving a therapeutic dose of said drug substance in a predetermined time; and wherein when administered said therapeutic dose is not exceeded in said predetermined time by ingestion of alcohol at biological pH.

Yet another embodiment is provided in a solid oral dose drug product comprising a mixture of polymorphic oxymorphone pamoate and oxymorphone xinafoate drug substances providing an immediate release therapeutic dosage of the oxymorphone from the pamoate within 30 minutes under gastric conditions and providing extended release of the oxymorphone from the oxymorphone xinafoate.

Yet another embodiment is provided in a solid oral dose dug product comprising a mixture of drug substances selected from the group consisting of codeine sulfate, codeine pamoate and codeine xinafoate providing immediate release therapeutic dosage of the codeine released from the codeine sulfate under gastric conditions, a pulsed dosage release corresponding to release of the codeine from the codeine xinafoate at a point from thirty minutes to three hours after ingestion, and an extended release of codeine from the codeine pamoate for patient treatment up to twenty-four hours after the drug product ingestion.

Yet another embodiment is provided in a solid oral dose drug product comprising a mixture of drug substances selected from levorphanol tartrate, levorphanol pamoate, and levorphanol xinafoate providing an immediate release therapeutic dosage of the levorphanol from the levorphanol tartrate under gastric conditions and an extended release of the levorphanol from levorphanol pamoate or xinafoate, the extended release providing therapeutic dosage up to twenty-four hours after ingestion by the patient.

### BRIEF DESCRIPTION OF THE FIGURES

FIG. 1 is the Differential Scanning calorimetry (DSC) diffractogram of amorphous oxymorphone pamoate.

FIG. 2 is the Fourier Transform Infrared (FTIR) spectrum of amorphous oxymorphone pamoate.

FIG. 3 is the Powder X-ray Diffraction (PXRD) diffractogram of amorphous oxymorphone pamoate.

FIG. 4 is the Proton Nuclear Magnetic Resonance (¹H NMR) spectrum of amorphous oxymorphone pamoate.

FIG. 5 is the Differential Scanning calorimetry (DSC) diffractogram of polymorphic oxymorphone pamoate.

FIG. 6 is the Fourier Transform Infrared (FTIR) spectrum of polymorphic oxymorphone pamoate.

FIG. 7 is the Powder X-ray Diffraction (PXRD) diffractogram of polymorphic oxymorphone pamoate.

FIG. 8 is the Proton Nuclear Magnetic Resonance (¹H NMR) spectrum of polymorphic oxymorphone pamoate.

FIG. 9 is the Differential Scanning calorimetry (DSC) diffractogram of oxymorphone xinafoate.

FIG. 10 is the Fourier Transform Infrared (FTIR) spectrum of oxymorphone xinafoate.

FIG. 11 is the Powder X-ray Diffraction (PXRD) diffractogram of oxymorphone xinafoate.

FIG. 12 is the Proton Nuclear Magnetic Resonance (¹H NMR) spectrum of oxymorphone xinafoate.

FIG. 13 is the Differential Scanning calorimetry (DSC) diffractogram of amorphous codeine pamoate.

FIG. 14 is the Fourier Transform Infrared (FTIR) spectrum of amorphous codeine pamoate.

FIG. 15 is the Powder X-ray Diffraction (PXRD) diffractogram of amorphous codeine pamoate.

FIG. 16 is the Proton Nuclear Magnetic Resonance (¹H NMR) spectrum of amorphous codeine pamoate.

FIG. 17 is the Differential Scanning calorimetry (DSC) diffractogram of codeine xinafoate.

FIG. 18 is the Fourier Transform Infrared (FTIR) spectrum of codeine xinafoate.

FIG. 19 is the Powder X-ray Diffraction (PXRD) diffractogram of codeine xinafoate.

FIG. 20 is the Proton Nuclear Magnetic Resonance (¹H NMR) spectrum of codeine xinafoate.

FIG. 21 is the Differential Scanning calorimetry (DSC) diffractogram of amorphous levorphanol pamoate.

FIG. 22 is the Fourier Transform Infrared (FTIR) spectrum of amorphous levorphanol pamoate.

FIG. 23 is the Powder X-ray Diffraction (PXRD) diffractogram of amorphous levorphanol pamoate.

FIG. 24 is the Proton Nuclear Magnetic Resonance (¹H NMR) spectrum of amorphous levorphanol pamoate.

FIG. 25 is the Differential Scanning calorimetry (DSC) diffractogram of polymorphic levorphanol pamoate.

FIG. 26 is the Fourier Transform Infrared (FTIR) spectrum of polymorphic levorphanol pamoate.

FIG. 27 is the Powder X-ray Diffraction (PXRD) diffractogram of polymorphic levorphanol pamoate.

FIG. 28 is the Proton Nuclear Magnetic Resonance (¹H NMR) spectrum of polymorphic levorphanol pamoate.

FIG. 29 is the Differential Scanning calorimetry (DSC) diffractogram of levorphanol xinafoate.

FIG. 30 is the Fourier Transform Infrared (FTIR) spectrum of levorphanol xinafoate.

FIG. 31 is the Powder X-ray Diffraction (PXRD) diffractogram of levorphanol xinafoate.

FIG. 32 is the Proton Nuclear Magnetic Resonance (¹H NMR) spectrum of levorphanol xinafoate.

FIG. 33 is the Differential Scanning calorimetry (DSC) diffractogram of amorphous naltrexone pamoate.

FIG. 34 is the Fourier Transform Infrared (FTIR) spectrum of amorphous naltrexone pamoate.

FIG. 35 is the Powder X-ray Diffraction (PXRD) diffractogram of amorphous naltrexone pamoate.

FIG. 36 is the Proton Nuclear Magnetic Resonance (¹H NMR) spectrum of amorphous naltrexone pamoate.

FIG. 37 is the Differential Scanning calorimetry (DSC) diffractogram of polymorphic naltrexone pamoate.

FIG. 38 is the Fourier Transform Infrared (FTIR) spectrum of polymorphic naltrexone pamoate.

FIG. 39 is the Powder X-ray Diffraction (PXRD) diffractogram of polymorphic naltrexone pamoate.

FIG. 40 is the Proton Nuclear Magnetic Resonance (¹H NMR) spectrum of polymorphic naltrexone pamoate.

FIG. 41 is the Differential Scanning calorimetry (DSC) diffractogram of naltrexone xinafoate.

FIG. 42 is the Fourier Transform Infrared (FTIR) spectrum of naltrexone xinafoate.

FIG. 43 is the Powder X-ray Diffraction (PXRD) diffractogram of naltrexone xinafoate.

FIG. 44 is the Proton Nuclear Magnetic Resonance (¹H NMR) spectrum of naltrexone xinafoate.

FIG. 45 is the graphical representation of the dissolution profiles for oxymorphone hydrochloride as a function of pH.

FIG. 46 is the graphical representation of the dissolution profiles for oxymorphone hydrochloride as a function of ethanol concentration.

FIG. 47 is the graphical representation of the dissolution profiles for amorphous oxymorphone pamoate as a function of pH.

FIG. 48 is the graphical representation of the dissolution profiles for amorphous oxymorphone pamoate as a function of ethanol concentration.

FIG. 49 is the graphical representation of the dissolution profiles for polymorphic oxymorphone pamoate as a function of pH.

FIG. 50 is the graphical representation of the dissolution profiles for polymorphic oxymorphone pamoate as a function of ethanol concentration.

FIG. 51 is the graphical representation of the dissolution profiles for oxymorphone xinafoate as a function of pH.

FIG. 52 is the graphical representation of the dissolution profiles for oxymorphone xinafoate as a function of ethanol concentration.

FIG. 53 is the graphical representation of the dissolution profiles for codeine sulfate as a function of pH.

FIG. 54 is the graphical representation of the dissolution profiles for codeine sulfate as a function of ethanol concentration.

FIG. 55 is the graphical representation of the dissolution profiles for amorphous codeine pamoate as a function of pH.

FIG. 56 is the graphical representation of the dissolution profiles for amorphous codeine pamoate as a function of ethanol concentration.

FIG. 57 is the graphical representation of the dissolution profiles for codeine xinafoate as a function of pH.

FIG. 58 is the graphical representation of the dissolution profiles for codeine xinafoate as a function of ethanol concentration.

FIG. 59 is the graphical representation of the dissolution profiles for levorphanol tartrate as a function of pH.

FIG. 60 is the graphical representation of the dissolution profiles for levorphanol tartrate as a function of ethanol concentration.

FIG. 61 is the graphical representation of the dissolution profiles for amorphous levorphanol pamoate as a function of pH.

FIG. 62 is the graphical representation of the dissolution profiles for amorphous levorphanol pamoate as a function of ethanol concentration.

FIG. 63 is the graphical representation of the dissolution profiles for polymorphic levorphanol pamoate as a function of pH.

FIG. 64 is the graphical representation of the dissolution profiles for polymorphic levorphanol pamoate as a function of ethanol concentration.

FIG. 65 is the graphical representation of the dissolution profiles for levorphanol xinafoate as a function of pH.

FIG. 66 is the graphical representation of the dissolution profiles for levorphanol xinafoate as a function of ethanol concentration.

FIG. 67 is the graphical representation of the dissolution profiles for naltrexone hydrochloride as a function of pH.

FIG. 68 is the graphical representation of the dissolution profiles for naltrexone hydrochloride as a function of ethanol concentration.

FIG. 69 is the graphical representation of the dissolution profiles for amorphous naltrexone pamoate as a function of pH.

FIG. 70 is the graphical representation of the dissolution profiles for amorphous naltrexone pamoate as a function of ethanol concentration.

FIG. 71 is the graphical representation of the dissolution profiles for polymorphic naltrexone pamoate as a function of pH.

FIG. 72 is the graphical representation of the dissolution profiles for polymorphic naltrexone pamoate as a function of ethanol concentration.

FIG. 73 is the graphical representation of the dissolution profiles for naltrexone xinafoate as a function of pH.

FIG. 74 is the graphical representation of the dissolution profiles for naltrexone xinafoate as a function of ethanol concentration.

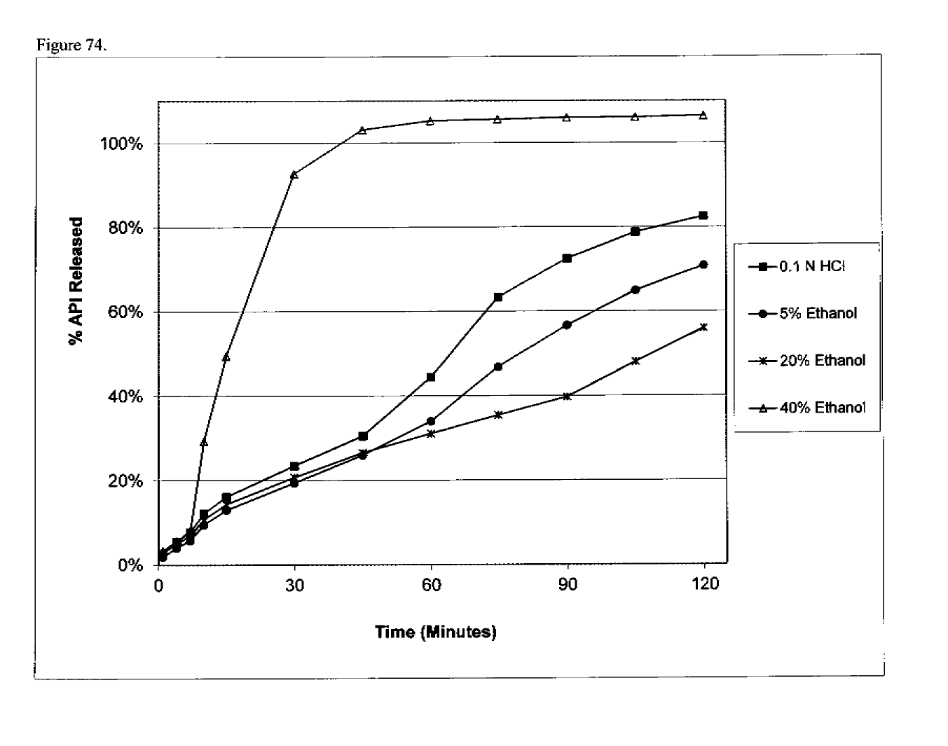

### DESCRIPTION

The instant invention is specific to drug substances, and drug products comprising the drug substance, wherein the drug substance is resistant to at least one of abuse or dose dumping. More specifically, the present invention is related to organic acid addition salts of oxymorphone, codeine, levorphanol and naltrexone which are less resistant to abuse or dose dumping. Even more specifically, the present invention is directed to amorphous and polymorphic forms of oxymorphone pamoate, oxymorphone xinafoate, codeine pamoate, codeine xinafoate, levorphanol pamoate, levorphanol xinafoate, naltrexone pamoate and naltrexone xinafoate.

Drug abuse has reached epidemic proportions. Drug product formulations to mitigate abuse, typically based on either retarding release of the drug substance from the drug product or by the use of an antagonist, have been proven to be totally ineffective since the drug substance is easily separated from the drug product in an activity known in the art as “free-basing”. The present invention is directed at efforts to mitigate abuse, and dose dumping, at the drug substance level. The new approach relies upon a more refined view of API solubility. Instead of the drug substance having high solubility in nearly all relevant media, the drug substance dissolution rate is modified to have a dissolution profile which interferes with intentional drug abuse.

For the purposes of the instant invention, two releases are defined. The first release is release of the drug substance from the drug product. This first release is typically sufficient to define the bioavailability of an active pharmaceutical since the second release is typically essentially instant. For the purposes of clarity, the first release would be release of a drug substance, oxymorphone pamoate for example, from the formulated drug product as a drug substance. The second release is the release of the active pharmaceutical of the drug substance from the salt form of the drug substance. Using oxymorphone pamoate as an example, the second release is the release of oxymorphone from the pamoate. Though not bound by theory, it is hypothesized herein that an active pharmaceutical, oxymorphone for example, is not bioavailable as the salt but must first be dissociated into the free oxymorphone as the active pharmaceutical. For salts typically utilized in the art this second step is essentially instantaneous.

A drug formulation which is selected for the prevention of drug abuse is specifically a drug substance which is bio-unavailable or not easily isolated if efforts to alter the intended or established route of administration are undertaken. In a preferred embodiment the active pharmaceutical of the drug substance is very slowly released from the drug substance under aqueous conditions at a pH of about 4 to about 9 and generates a solid of an organic acid at pH below about 4. At pH above about 9, the organic acid as its alkali, alkali earth, or transition metal salt and the amine containing active pharmaceutical ingredient, as its free base, are sufficiently soluble as to prevent separation of the components and thus inhibiting direct isolation of the API, as its free base, without additional processing.

In contrast to the prior art, it is relevant to the present invention to note the importance of pH in controlling the release of the active pharmaceutical from the drug substance to achieve absorption and consequently, the medicinal effect. The pH of the gastrointestinal tract essentially remains highly acidic, such as below a pH of 3, with the exception of the lower colon which reaches pH 8; vaginal pH is typically around 5.8 and the nasal cavity is approximately pH 4.5. More generally, each of the mucosal surfaces, particularly ocular, nasal, pulmonary, buccal, sublingual, gingival, rectal and vaginal are receptive to drug absorption if release can occur. A dominating feature of the present invention is the severely retarded release of the active pharmaceutical from the drug substance in the pH range of about 4 to 9 which encompasses the physiological pH of the mucosa. These release properties were an unexpected finding recognized and observed after performing dissolution tests over a wide pH range on several unrelated compounds. The release properties and solubility profiles are a means to evaluate a reasonable dosage application to the mucosa. The non-release of the active pharmaceutical from the drug substance in the 4 to 9 pH range negates absorption and prevents the physical act of abuse. For the amine-containing hydrochloride salts, an abuse mechanism remains operative since these salts do not exhibit the discriminating “on/off” switch of the present invention as the active pharmaceutical is readily released as the hydrochloride salt at all pH's.

The pamoate salts are quite insoluble in water and highly resistant to extraction techniques. With respect to hydrocodone salts, a simple comparison of the bitartrate and pamoate, confirms this abuse deterrent feature. At 25° C. hydrocodone bitartrate has water solubility of greater than 126 mg/mL while the pamoate salt has water solubility of only 0.916 mg/mL. For all practical purposes, crushing, grinding or milling a drug product containing hydrocodone bitartrate and then subjecting the material to water extraction will lead to isolation of the active ingredient. In contrast, similar treatment of drug product containing hydrocodone pamoate would result in very poor yields and hence hydrocodone pamoate would be unfavorable for abuse purposes. Beyond water extraction are free-basing and solvent partitioning extractions. Here too, the pamoate favors formation of intractable gels, and gums and the use of non-aqueous solvents for partitioning purposes creates complexity and ultimately, yield loss. The yield loss issue is important in constructing abuse deterrent products. In the case of the opioids, the active ingredient is available in relatively low amounts per tablet due to the high pharmacological activity of the drug. Even with extended release drug products which can contain drug substance amounts sufficient for twelve or twenty-four hour intervals between dosing, yield loss becomes significant when the pamoate drug substance salt is employed.

The water insolubility of pamoate and xinafoate drug substance salts, and their often slow release around neutral and basic pH conditions, make these drug substances nearly impossible to abuse by mucosal membrane administration. The pamoates and xinafoates are highly resistant to dose dumping and analogously, alcohol is therefore a poor solvent choice for extracting pamoate and/or xinafoate based actives from formulated drug products.

The unexpected and novel characteristics of the pamoate and xinafoate drug substance salts, in conjunction with their unique amorphous and polymorphic release rate responses, allows for nearly perfect application to abuse deterrent medications. In addition to imparting tamper/extraction resistance, and essentially removing drug substance physiological activity when applied to mucosal membranes, an abuser's attempt to inject the insoluble pamoate or xinafoate drug substance salts would fail. Poor extraction efficiency, lack of absorption effectiveness to abuse routes of administration, and pH dependent release profiles of the pamoate and xinafoate drug substance salts provide abuse deterrent properties while providing safe and efficacious medication to a patient in need of such treatment if the drug product is used as intended.

Design features available to achieving abuse deterrent properties at the drug substance level and not necessarily through drug product formulation techniques include: a) choice of organic acid family from which to make a salt of the amine-containing active ingredient, b) the stoichiometric choices available for salt formation, c) the amorphous or crystalline form of the salt, and even perhaps d) water content or residual amounts of inorganic species left in the compound after processing. Herein, the xinafoates and pamoates were selected as the salt family. Salts based on beta-oxy-naphthoic acid (BON acid), also known as xinafoate salts, are formed. They are 1:1 salts of amine-containing active ingredient: BON component. In contrast, salts based on pamoic acid optionally form 1:1 salts or 2:1 salts. The 2:1 pamoate salts are addressed herein and represent two equivalents of amine-containing active ingredient for every mole of pamoate component. The literature is replete with suggestion and examples of providing both the 1:1 and 2:1 pamoate salts; however, few exemplars of 1:1 salts are available despite the claims. Many have desired that stoichiometric adjustment of the reagents is sufficient to obtain the 2:1 or the 1:1 pamoate salts, but nature is far from that accommodating. Often the 1:1 pamoate salts presumed to have been prepared are not substantiated by corresponding analytical data confirming the stoichiometry, or the functionality of the “open” position remaining on the pamoate moiety. The unreacted site may be as an ionized carboxylate or may be present as the free acid, but structurally, this substituent functionality is left ambiguous. Such an ambiguity has significant impact on the performance features of the theorized 1:1 drug substance pamoate salt and would likely impact release properties. Most certainly, for reasonable commercial development, the functionality must be defined even if it is a defined mixture of carboxylic acid and carboxylate salt.

Naltrexone hydrochloride, which is the N-cyclopropylmethyl derivative of oxymorphone, is utilized as an opioid receptor antagonist. As such, its use in combination with orally administered, extended release morphine sulfate has been commercialized in the combination product Embeda®. While the product was recalled in 2011, the technological intent was to provide an abuse deterrent form of morphine. The Embeda® capsules were formulated with morphine sulfate and an indigestible inner core containing the naltrexone hydrochloride. The concept was a means to provide morphine in an extended release format, but should the product be crushed for inhalation or intravenous injection, the drug abuser would be denied their “high” by the competitive binding properties of naltrexone to the exclusion of morphine. Herein, organic acid addition salts of naltrexone have been disclosed for their compatibility in drug product formulations containing drug substance salts also derived from identical families of organic acids. While much of the discussion focuses on abuse deterrence through use of specific drug substances, the inclusion of naltrexone derivatives is to support combination drug product development wherein two or more methodologies of abuse deterrence are incorporated into the dosage product. The invention herein provides compounds possessing abuse deterrence, but which can be employed under specific circumstances to give immediate release, extended release and controlled release drug product characteristics wherein the release of the drug substance from the drug product is controlled. The disclosed compounds are also amenable to various processing techniques to achieve selected release profiles without compromising the abuse deterrent properties. By way of example, an enteric coating on drug products containing drug substances prepared as the organic acid addition salts of amine containing active ingredients, such as the drug substance pamoates and xinafoates, provides products for targeted release in the bowel, and with dosage strengths suitable for immediate release or extended release wherein the drug substance contained therein is not susceptible to abuse.

Preparation of amorphous and polymorphic forms of oxymorphone pamoate, oxymorphone xinafoate, codeine pamoate, codeine xinafoate, levorphanol pamoate, levorphanol xinafoate, naltrexone pamoate and naltrexone xinafoate was directed toward imparting unique physical and chemical properties, and performance features to these drug substances. Subsequent inclusion of the drug substance into a dosage formulation to produce a drug product transfers these unique properties and features to the drug product. The organic acid addition salts of the well-known active ingredients have been demonstrated to provide the pathway for achieving performance features such as drug substance release profiles otherwise unavailable from the traditional and commercially available mineral acid salts or highly water soluble tartrate salts.

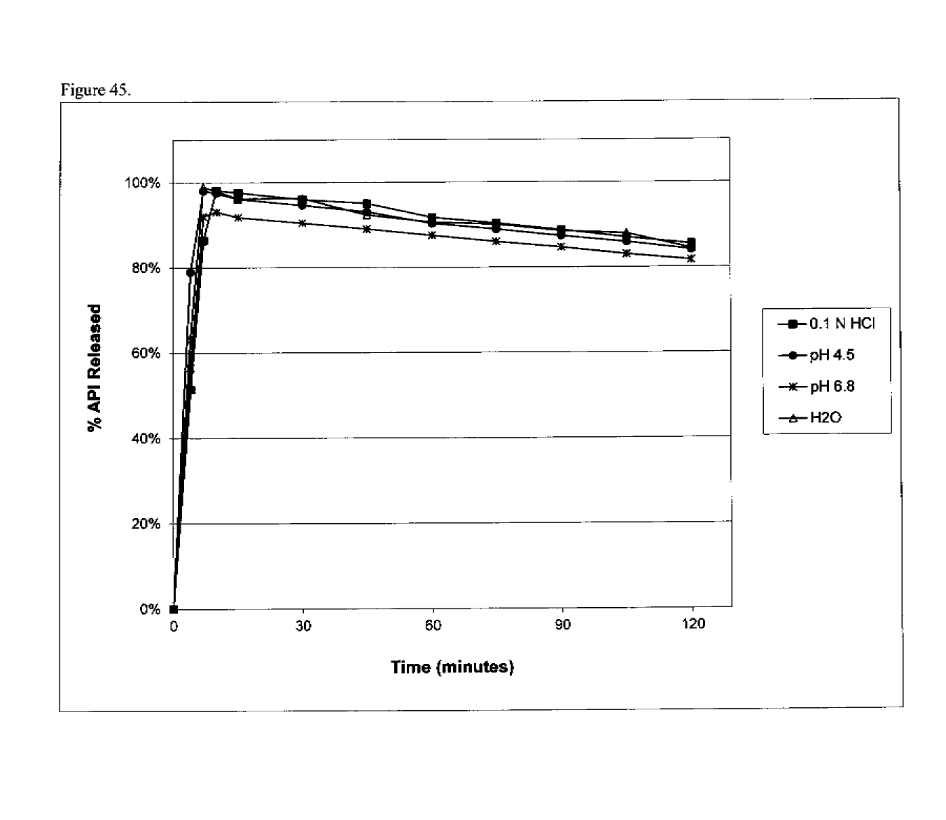

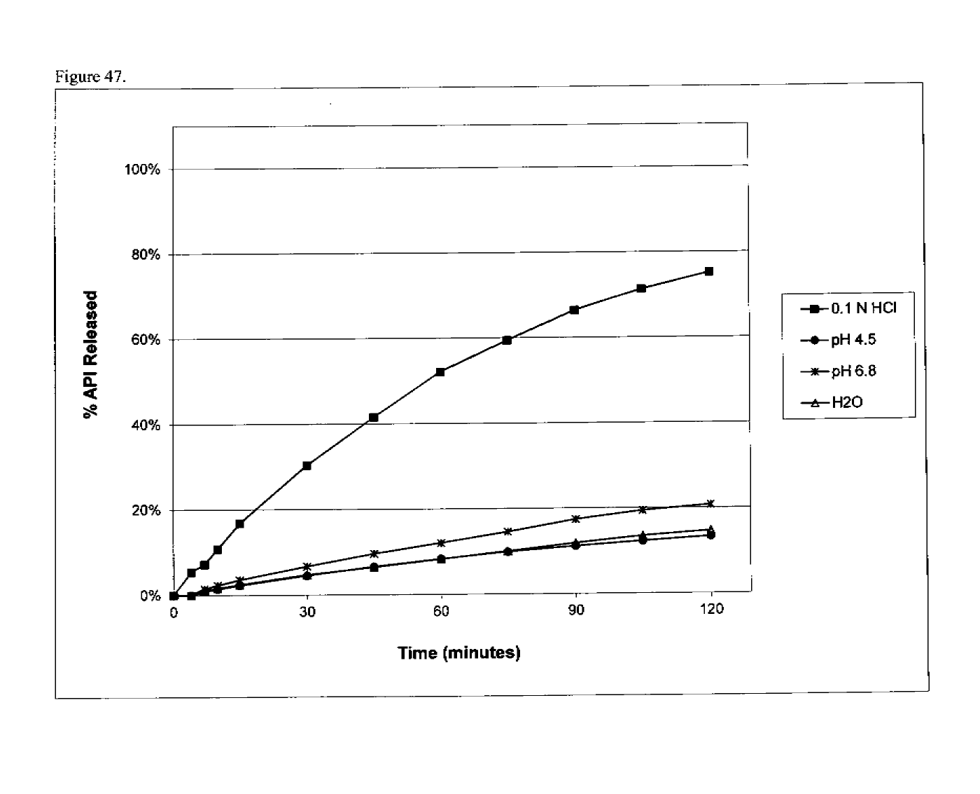

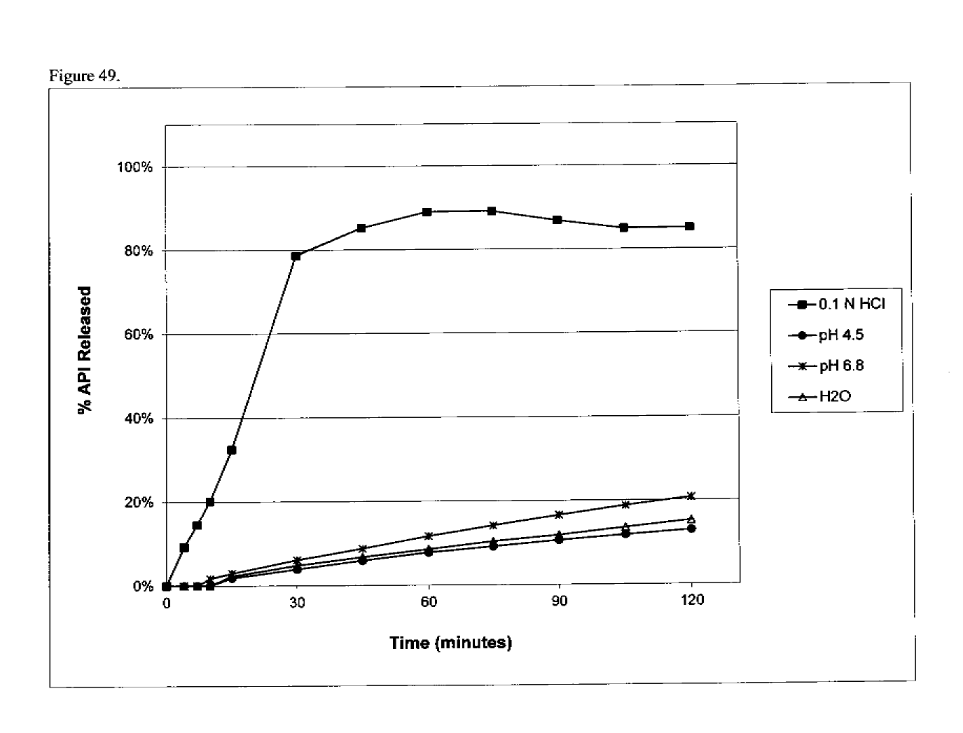

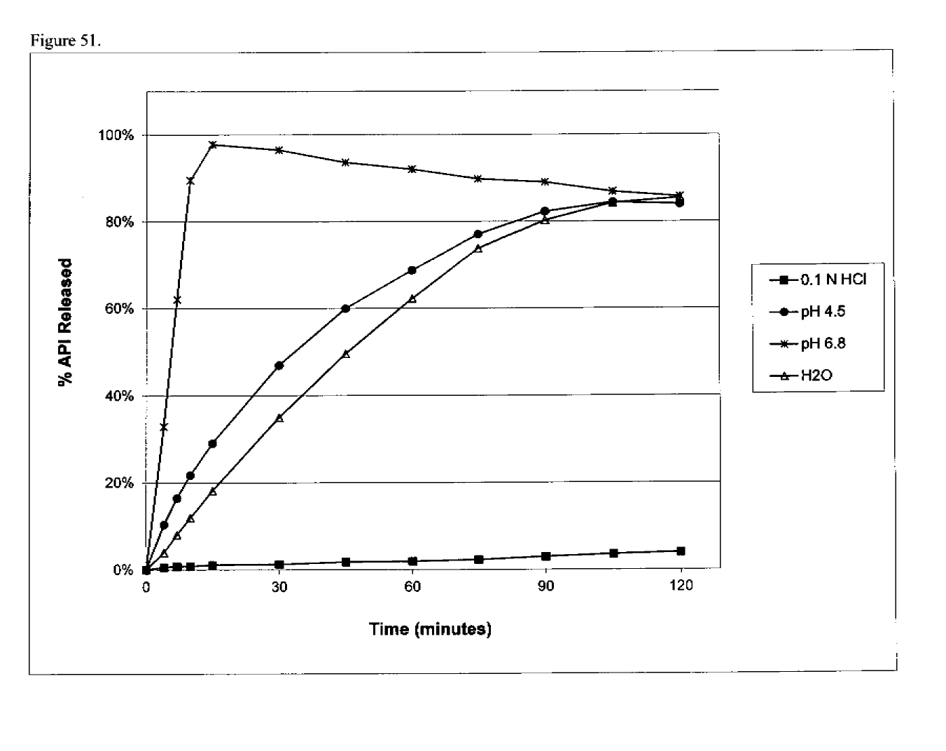

By way of example, the pH dependent dissolution profile of oxymorphone HCl drug substance is illustrated in FIG. 45. As a drug substance, the hydrochloride salt is highly soluble under a broad range of pH conditions, and unto itself, is consistent with the traditional drug substance design features of possessing an immediate release profile. Oxymorphone hydrochloride exhibits an immediate release drug dissolution profile after only fifteen minutes. In contrast, the organic acid addition salts offer dissolution profiles suitable for alternative dosing regimens of release. For instance, amorphous oxymorphone pamoate has a pH dependent dissolution profile as summarized in FIG. 47. The amorphous pamoate salt behaves quite differently than the analogous hydrochloride salt in that less than 30% of the oxymorphone active pharmaceutical is released from the salt form, as the drug substance, in less than thirty minutes under all pH conditions. In contrast, the polymorphic form of oxymorphone pamoate has a different pH dissolution profile than its amorphous analog as illustrated in FIG. 49, and illustrates a dissolution anomaly often observed with the pamoate salts of active ingredients. For the 0.1 N HCl dissolution condition, polymorphic oxymorphone pamoate actually has a faster dissolution profile than the amorphous form; the observation is contrary to traditional pharmaceutical practice wherein the amorphous form of a drug substance is desired because of a faster dissolution rate than the crystalline form. However, the polymorphic and amorphous forms of oxymorphone pamoate have a similar dissolution profile in water, pH 4.5 and pH 6.8 dissolution test conditions. The dissolution profile of oxymorphone xinafoate has an intriguing aspect as can be seen in FIG. 51. The xinafoate unexpectedly exhibits little-to-no release under the 0.1 N HCl condition despite the expectation salt dissociation would occur under this condition. Further, the xinafoate exhibited immediate release characteristics for the pH 6.8 condition.

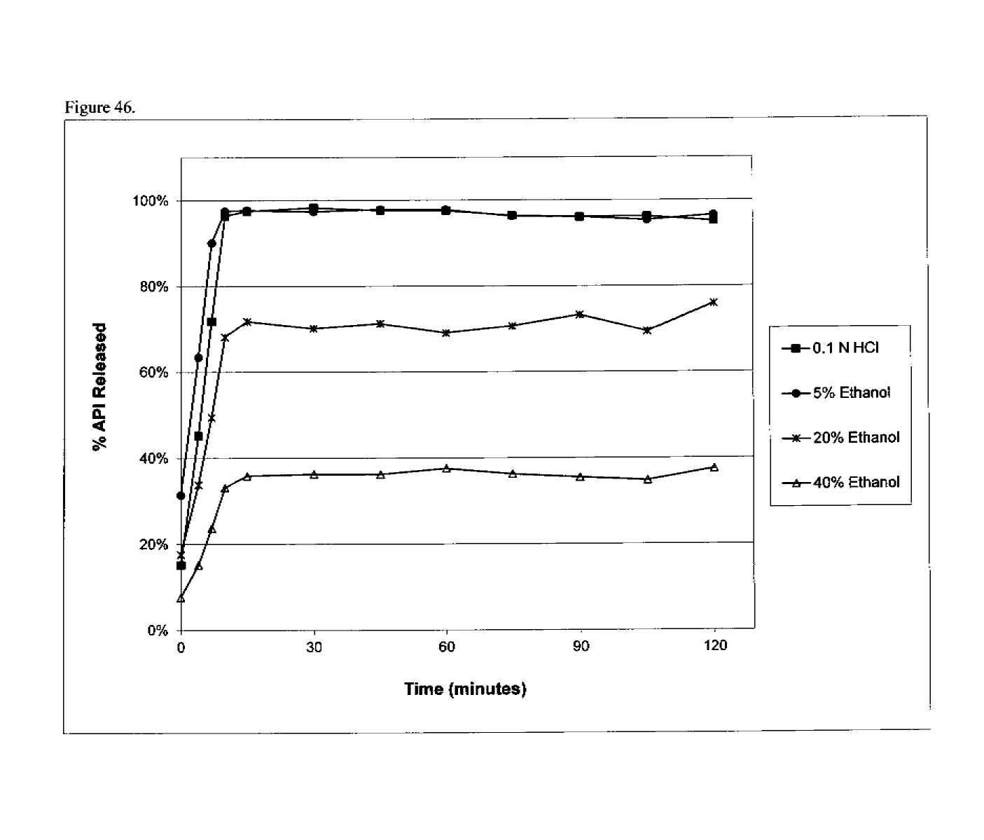

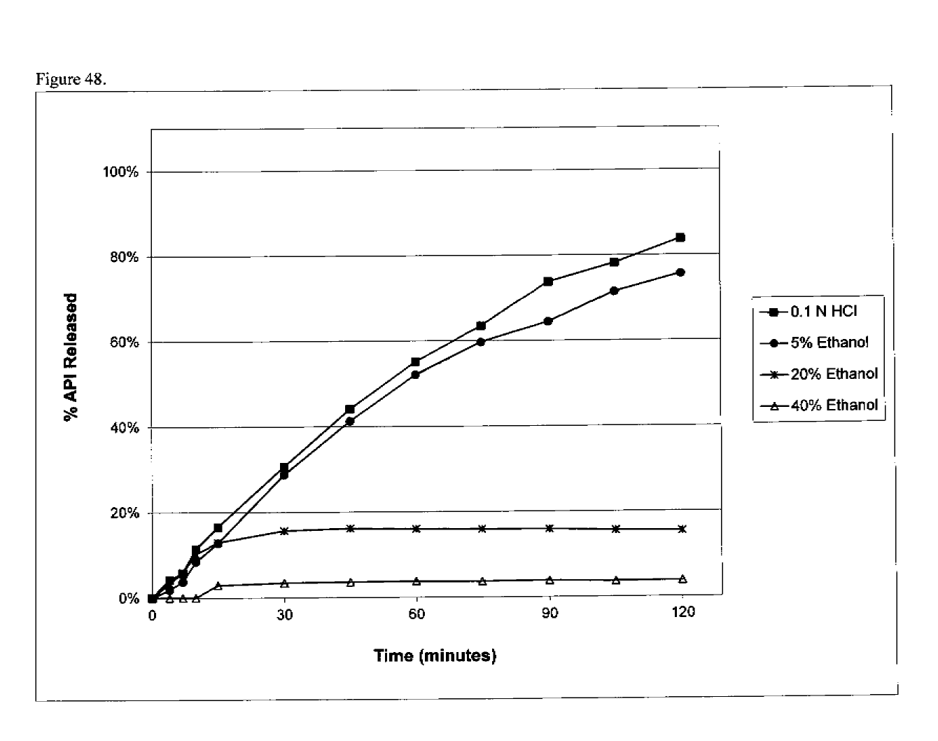

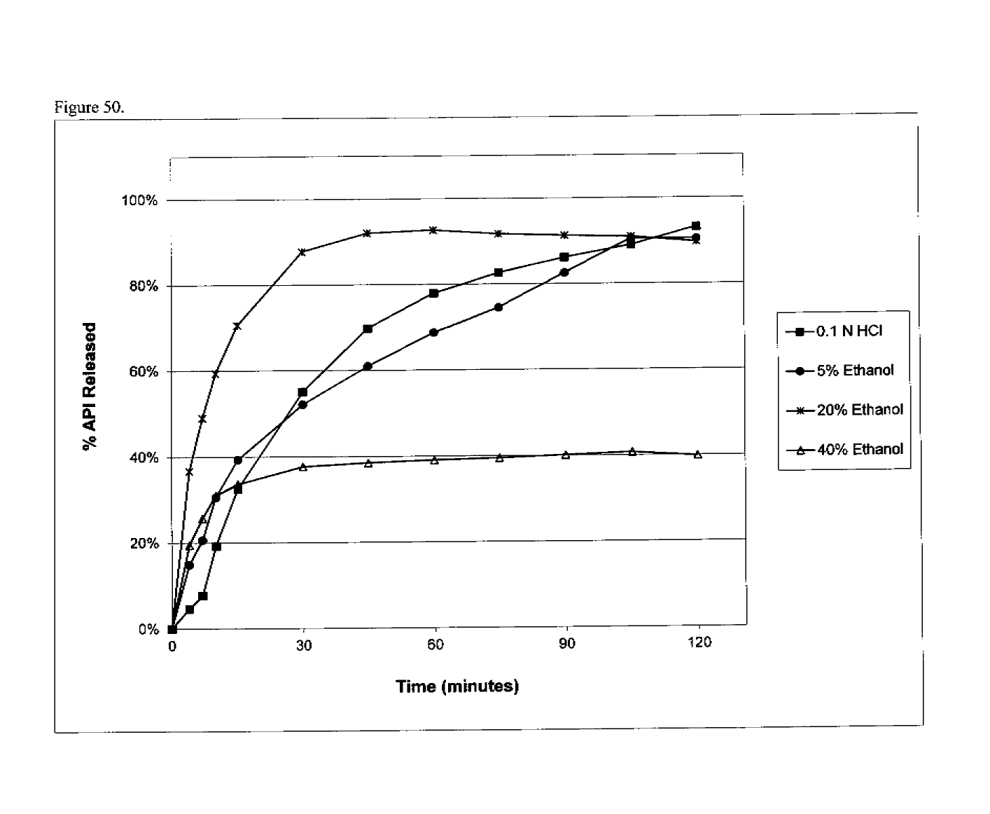

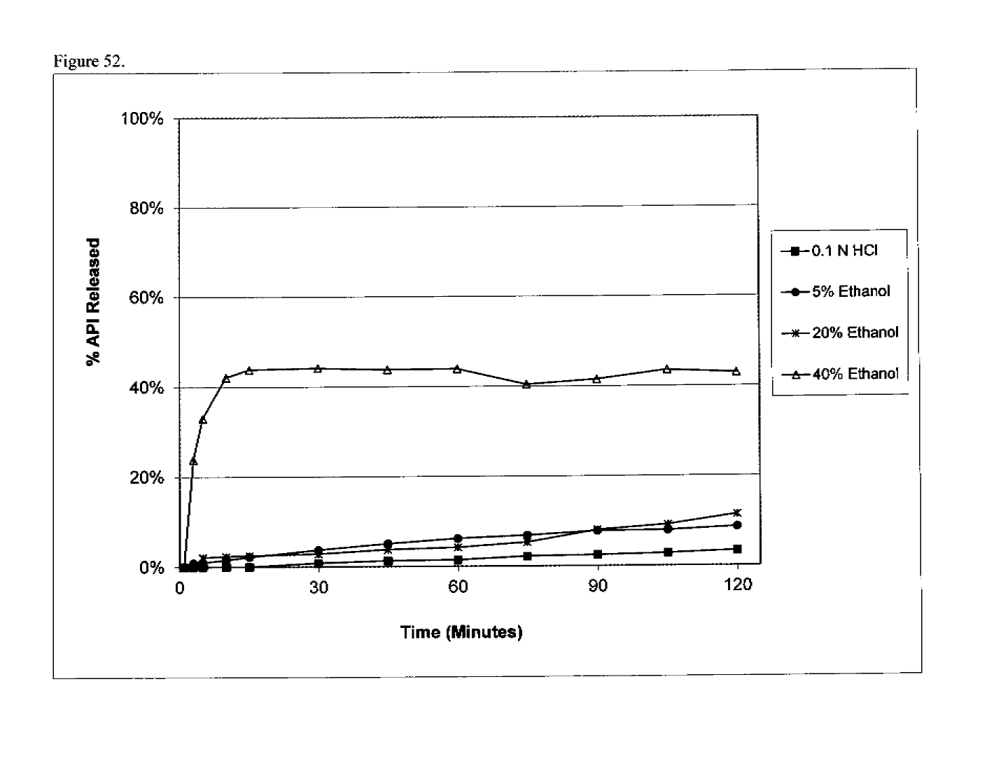

The oxymorphone salts were evaluated for their propensity toward dose dumping and were evaluated according to a dissolution test procedure wherein the sample is subjected to 0.1 N HCl with progressive increases in ethanol concentration. Clearly, oxymorphone HCl exhibits dose dumping properties under the 0.1 N HCl condition, as illustrated in FIG. 45, and when in the presence of the acid containing 5% ethanol as illustrated in FIG. 46. However, increasing levels of ethanol appear to decrease the overall solubility of oxymorphone and hence leads to a slower dissolution rate. The dose dumping evaluation is performed to mimic what an intended abuser would want to accomplish—the use of alcohol, co-ingested with the drug product—to release the active ingredient quickly and achieve a “high”. Inspection of FIG. 48, which summarizes the dose dumping dissolution profile of amorphous oxymorphone pamoate, demonstrates how a potential abuser would be highly frustrated at attempts to dose dump the active pharmaceutical, oxymorphone, from its pamoate salt. Even after thirty minutes, at most only about 30% of the active pharmaceutical is released from the drug substance under the “best” conditions, such as just stomach acid, or with 5% ethanol present such as expected from drinking the equivalent of about a beer. However, it can be understood from FIG. 50 that utilization of polymorphic oxymorphone pamoate in a drug product might provide a product which could be abused, or misused, through dose dumping. In the presence of an acidic 20% ethanol environment, the polymorphic oxymorphone pamoate appears to dose dump similarly to the traditional oxymorphone hydrochloride drug substance but, under the other conditions, only modest dose dumping occurs. For oxymorphone xinafoate the dose dumping profile is summarized in FIG. 52 and nearly under all conditions there is no chance of dose dumping the active ingredient as extended release is indicated, but strangely, its solubility under the 40% ethanol condition resembles that of the corresponding hydrochloride salt.

For the oxymorphone series of salts, the screening techniques of pH dependent and dose dumping dissolution profiles provide for interesting observations and conclusions. In order to produce an abuse deterrent drug product, the amorphous oxymorphone pamoate appears to be the best choice principally because it does not dose dump, and an extended release (ER) drug release profile is available as particularly noted for the 0.1 N HCl condition, which is approximately pH 1 and corresponding to stomach pH. The organic acid addition salt approach also demonstrates an important refinement to the prior art. Historically, the literature has implied that pamoates are useful for providing slow release salts of active ingredients. This observation, which may have appeared true at that point and time of pharmaceutical development and understanding, is not a universal truth regarding the pamoates. For instance, most formulators would agree that polymorphic oxymorphone pamoate could be easily formulated to produce an immediate release drug product given the 0.1 N HCl condition release rate for this compound. However as stated above, and shown in FIG. 49, the amorphous analog would be well suited for an extended release product given the 0.1 N HCl condition release rate shown in FIG. 47. Contrary to expectations from the art, the polymorphic oxymorphone pamoate has a faster dissolution rate than the amorphous form and is sufficient for use in an immediate release drug product. The amorphous oxymorphone pamoate, which, by definition does not contain an ordered crystalline structure requiring a higher enthalpy of dissolution and hence should exhibit a faster dissolution rate than its polymorphic analog, is instead suitable for an extended release dosage product independent of formulation techniques to alter the dissolution profile. Clearly, there is substantially more to the story than that promulgated by the prior art and further, the use of polymorphic differentiation provides the potential for enhanced drug product performance features including but not limited to abuse deterrence and assorted release profiles.

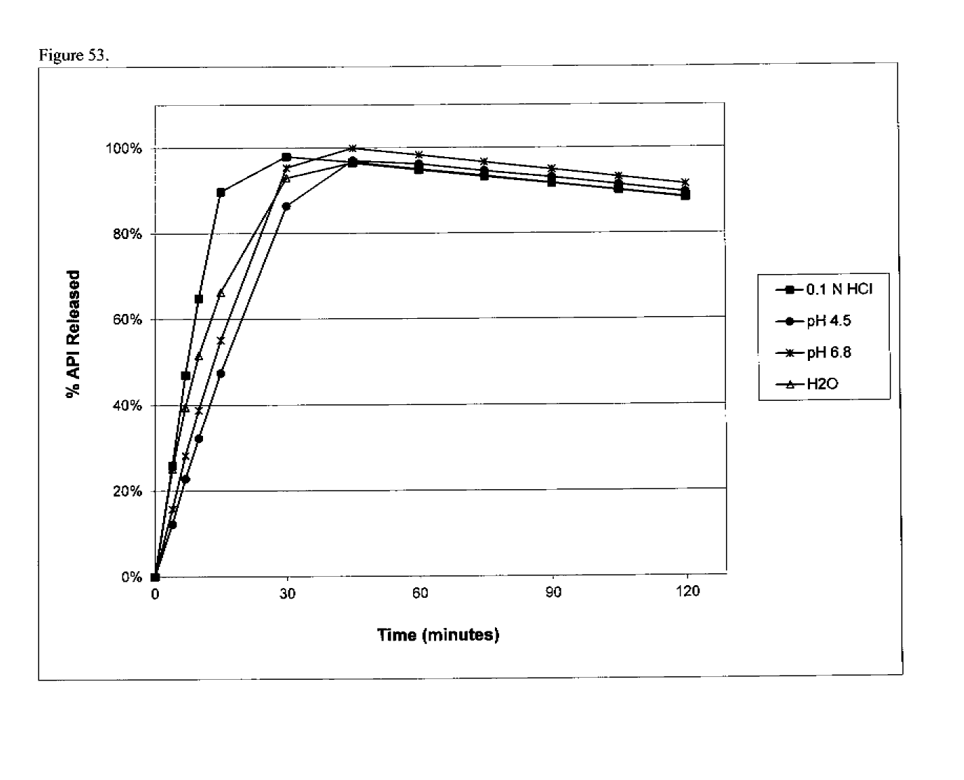

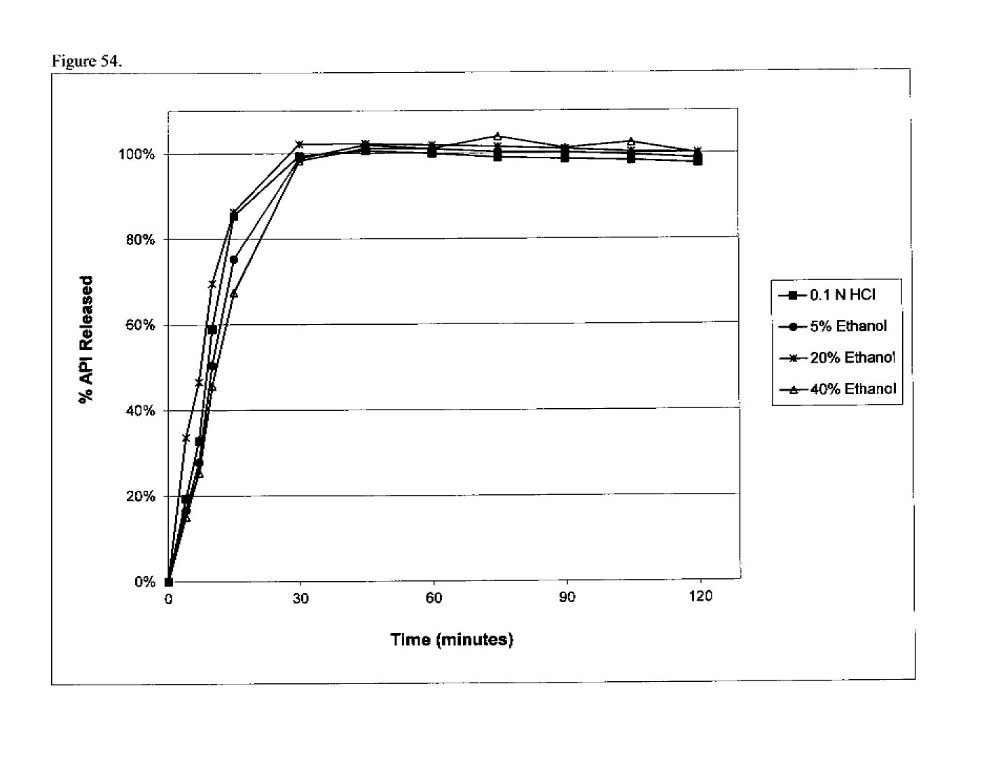

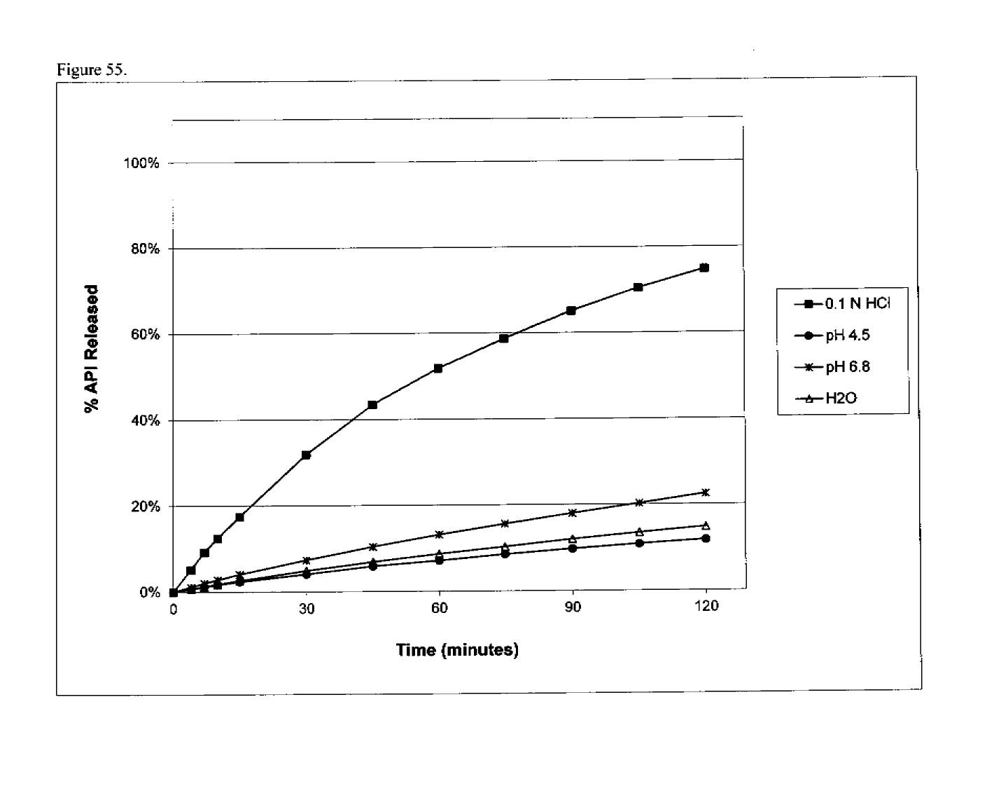

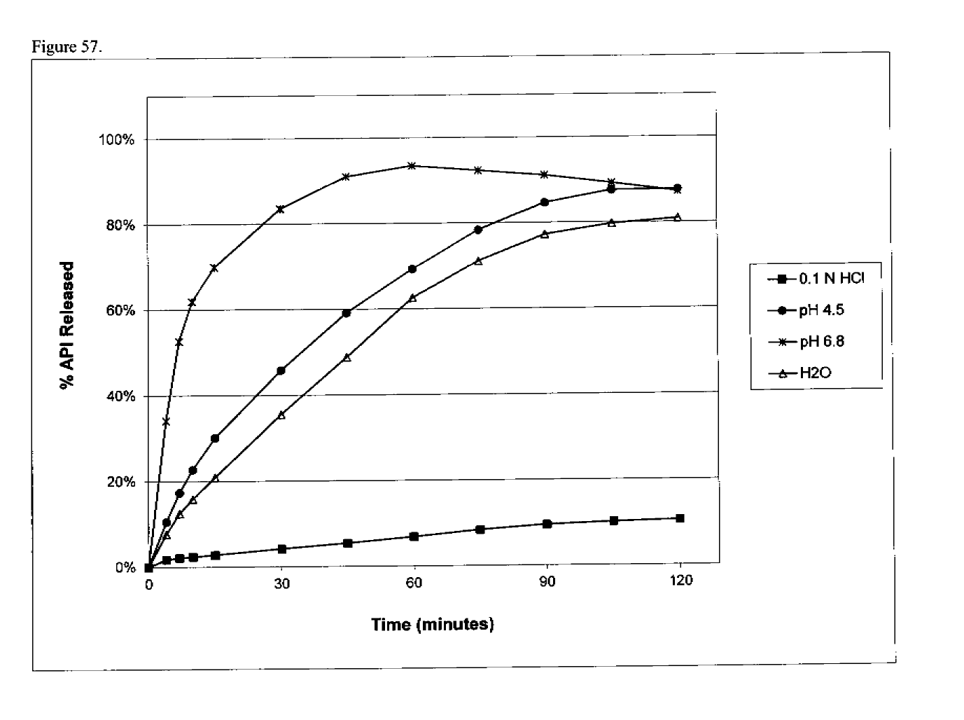

The codeine salt series was not absent of dissolution profile anomalies either when comparing codeine's mineral acid sulfate salt with the organic acid addition salts. The pH dependent dissolution profile of codeine sulfate is summarized in FIG. 53 wherein, not surprisingly, codeine sulfate exhibits an immediate release profile at all pH conditions. FIG. 54 summarizes the dose dumping dissolution profile of codeine sulfate and here too, it dose dumps under all conditions of acidic ethanol. Contrarily, amorphous codeine pamoate has a pH dissolution profile as summarized in FIG. 55 and exhibits an extended release profile under the 0.1 N HCl condition. At the other conditions, amorphous codeine pamoate's dissolution rate is highly attenuated in stark contrast to the corresponding sulfate salt. Codeine xinafoate has a surprising anomaly in its pH dependent dissolution profile in that the xinafoate exhibits an immediate release profile at the pH 6.8 condition, an extended release profile at pH 4.5 and in water, and like oxymorphone xinafoate, very poor release under pH 1 conditions as seen in FIG. 57. Similarly, and discussed in more depth below, levorphanol xinafoate also has a highly attenuated (defined as less than twenty percent release of the active ingredient) release profile at pH 1. There is an advantage available associated with this peculiar observation related to xinafoate salts since for products based on drug substance xinafoates, the principal location of active ingredient release would be in the bowel and not in the stomach.

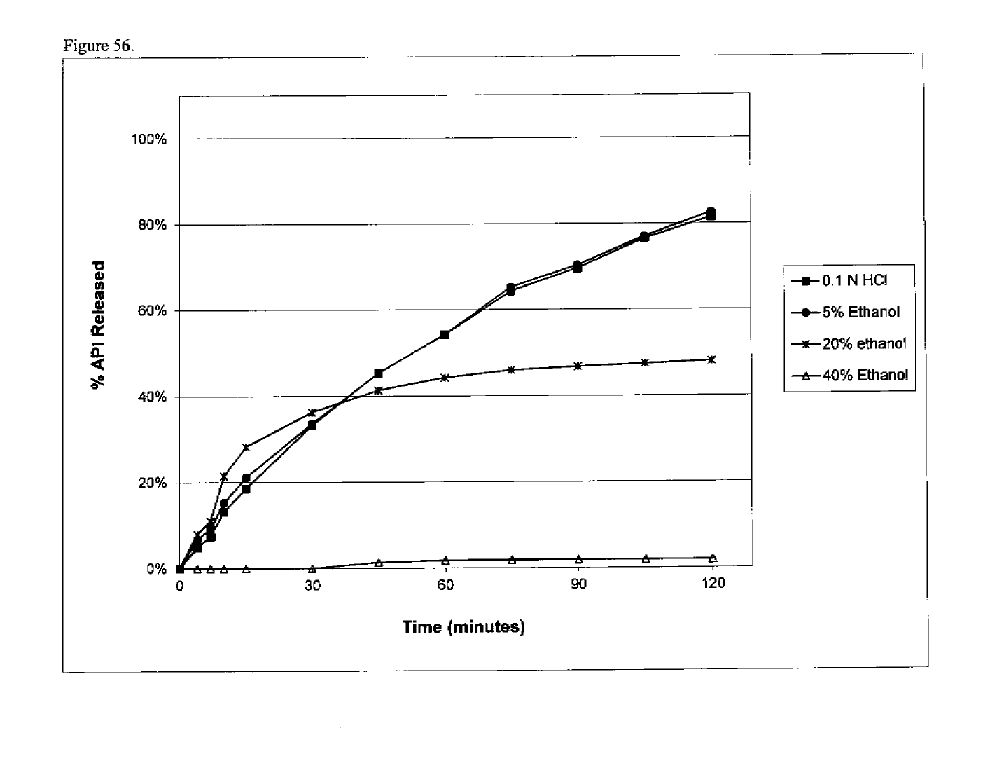

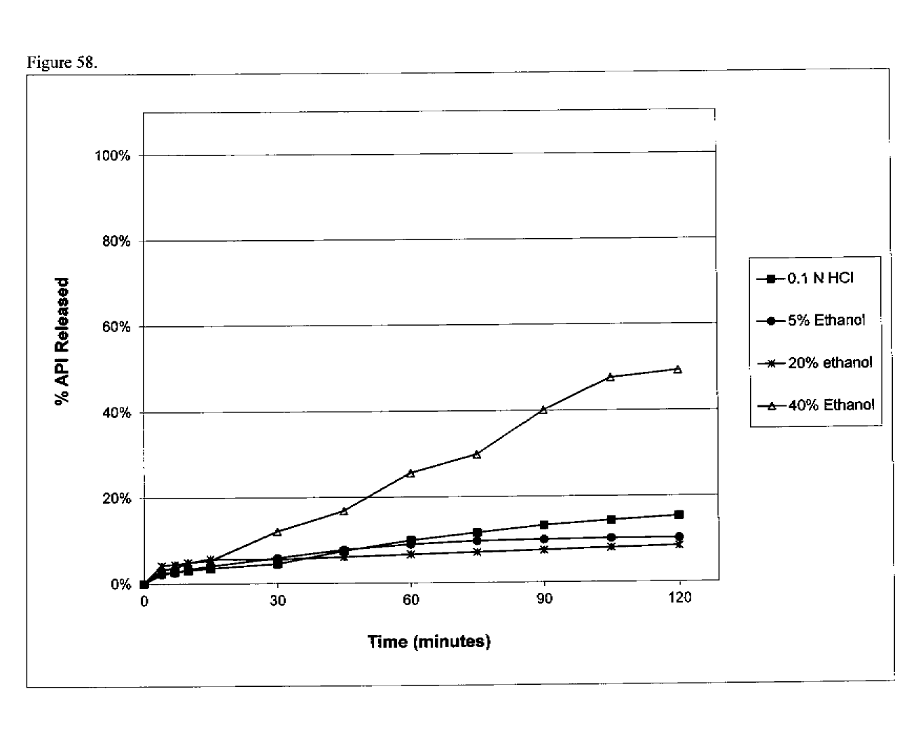

With respect to dose dumping, codeine pamoate and codeine xinafoate are both resistant to dose dumping. FIG. 56 summarizes the dose dumping profile of amorphous codeine pamoate wherein under any condition, no more than 40% of the active ingredient is released after thirty minutes. With the xinafoate salt, less than about 10% of the active is released within thirty minutes as illustrated in FIG. 58. For a preferred drug substance selection for a codeine based drug product, codeine pamoate would offer the best in an extended release drug product presentation possessing abuse deterrent characteristics.

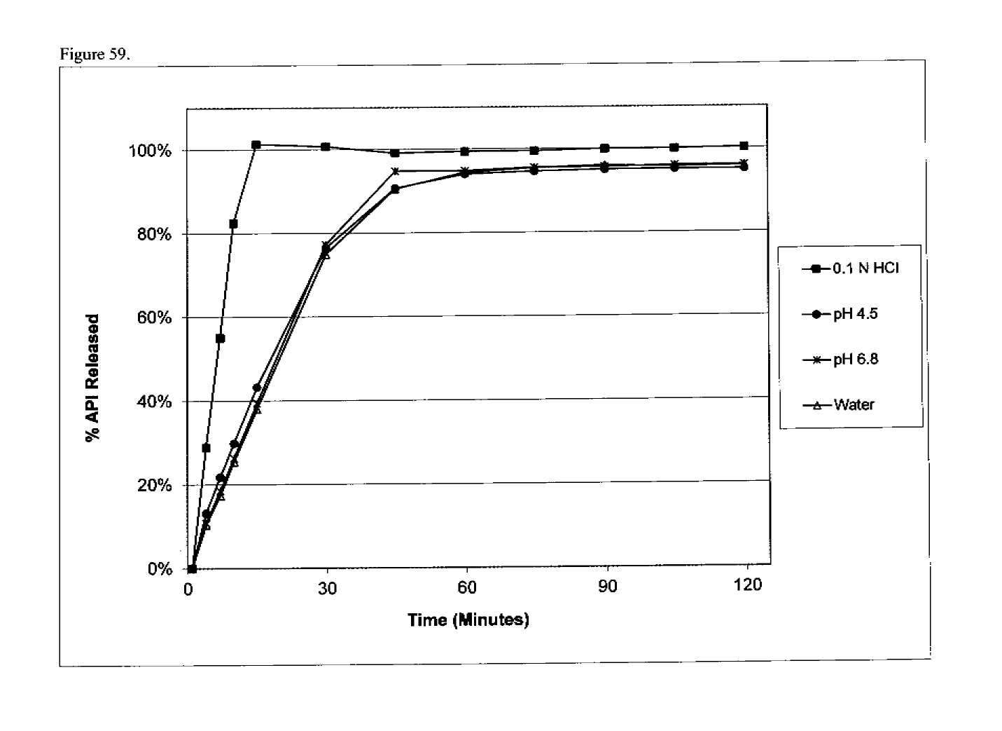

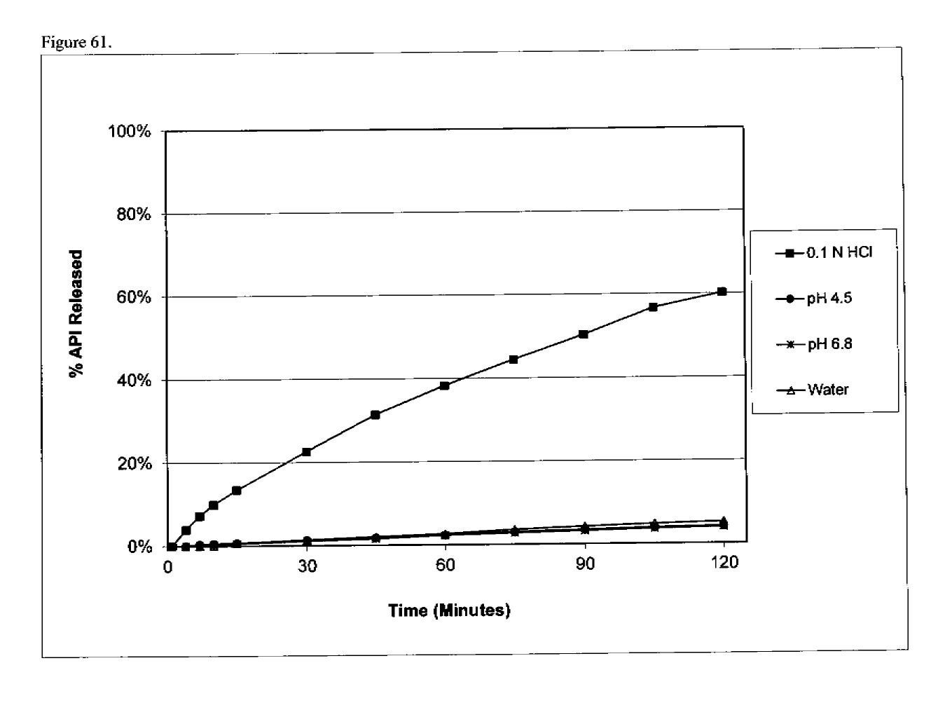

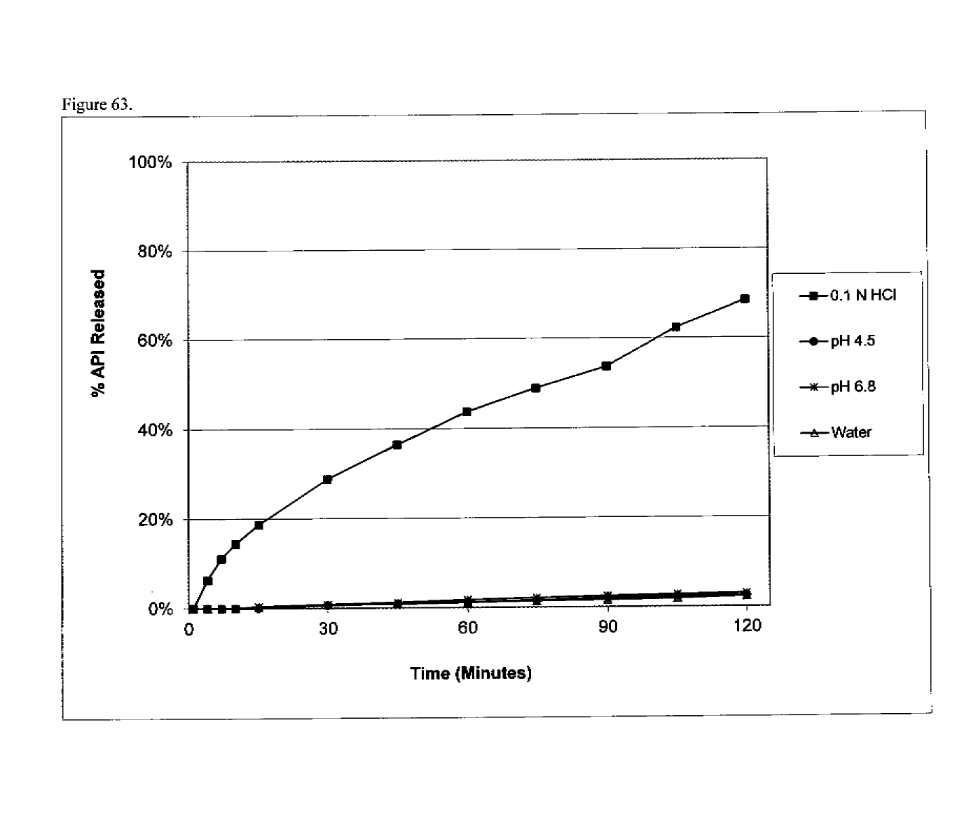

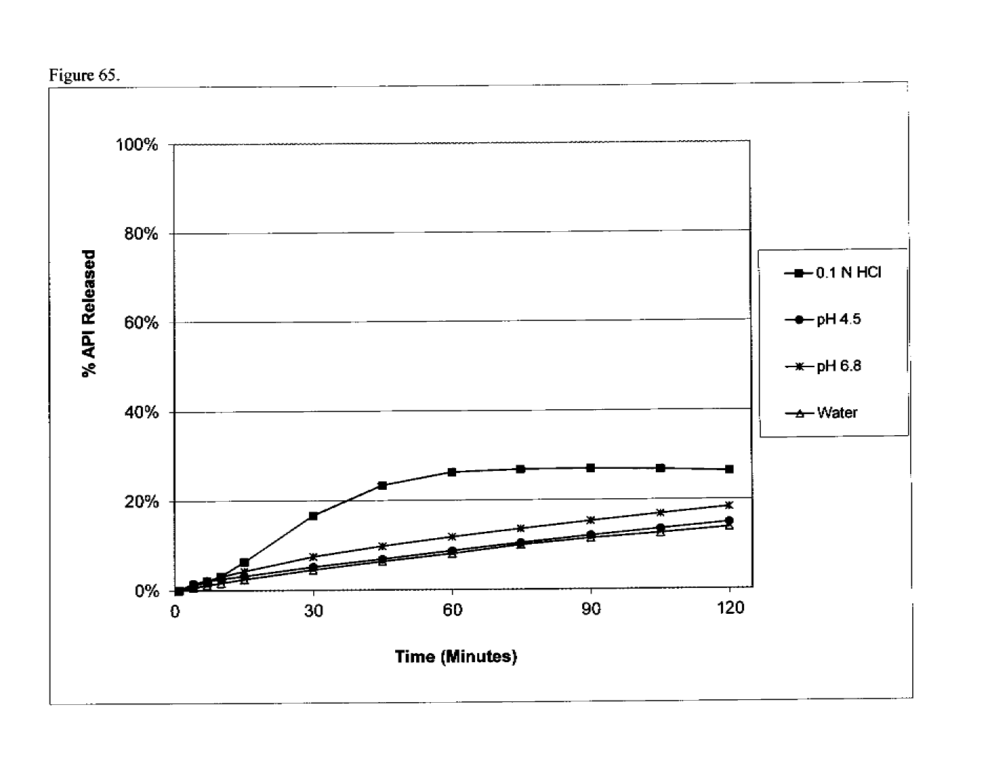

FIG. 59 summarizes the pH dependent dissolution profile of levorphanol tartrate. As can be seen, levorphanol tartrate exhibits an immediate release profile in 0.1 N HCl and about 80% release after 30 minutes in water, and in 4.5 or 6.8 pH media. True to form, traditional drug substance design, and for all practical purposes other than abuse deterrence, levorphanol tartrate exhibits an immediate release. FIG. 61 and FIG. 63 summarize the pH dependent dissolution profiles of amorphous levorphanol pamoate and polymorphic levorphanol pamoate, respectively. While there is little morphic differentiation observed by comparing these two figures, the pamoates exhibit quite a different dissolution profile than the tartrate. The pamoates release only about 30% of the active from the drug substance after thirty minutes for the 0.1 N HCl condition and considerably less under the remaining pH conditions represented by water, 4.5 or 6.8 pH media. In contrast levorphanol tartrate has an immediate release profile. FIG. 65 illustrates the pH dependent dissolution profile of levorphanol xinafoate and the release of levorphanol from the xinafoate is highly attenuated, less than 20%, under all pH conditions.

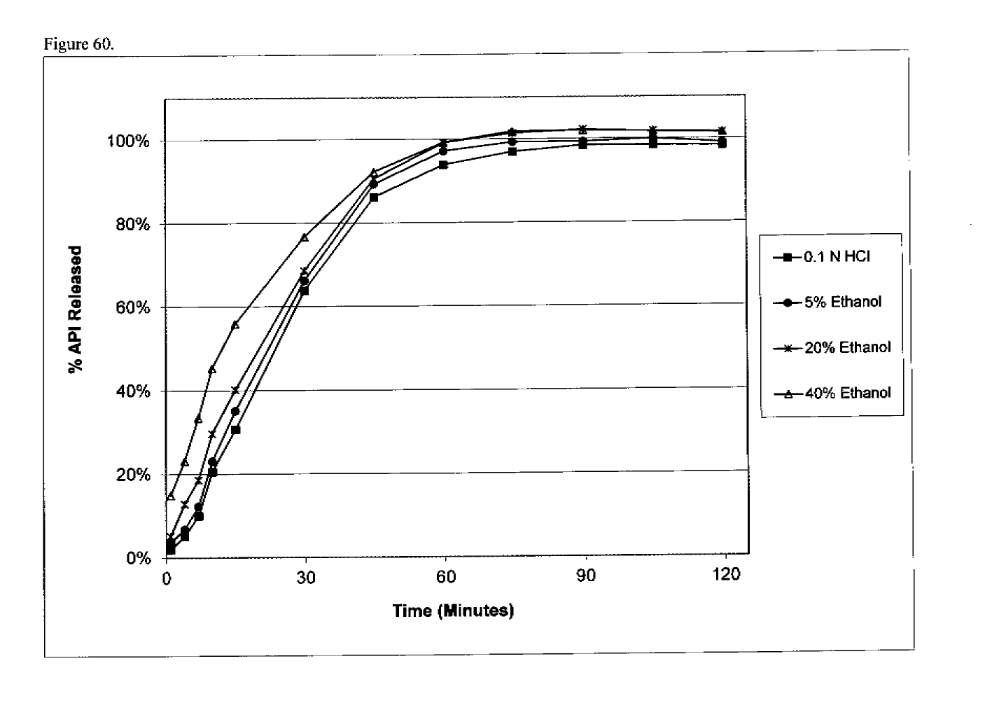

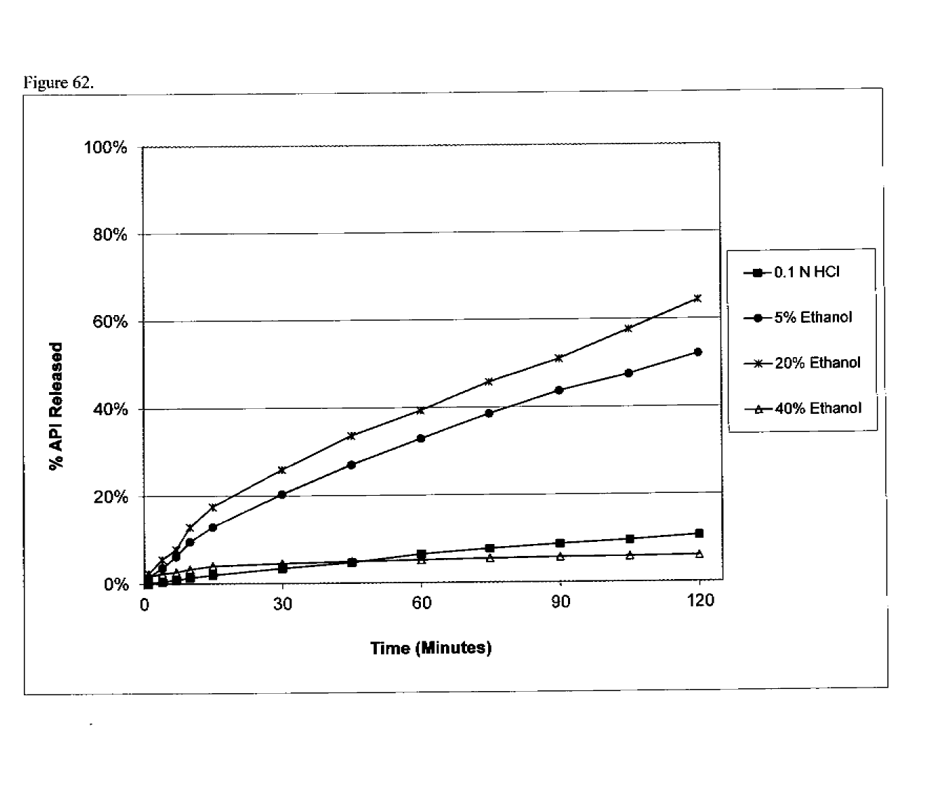

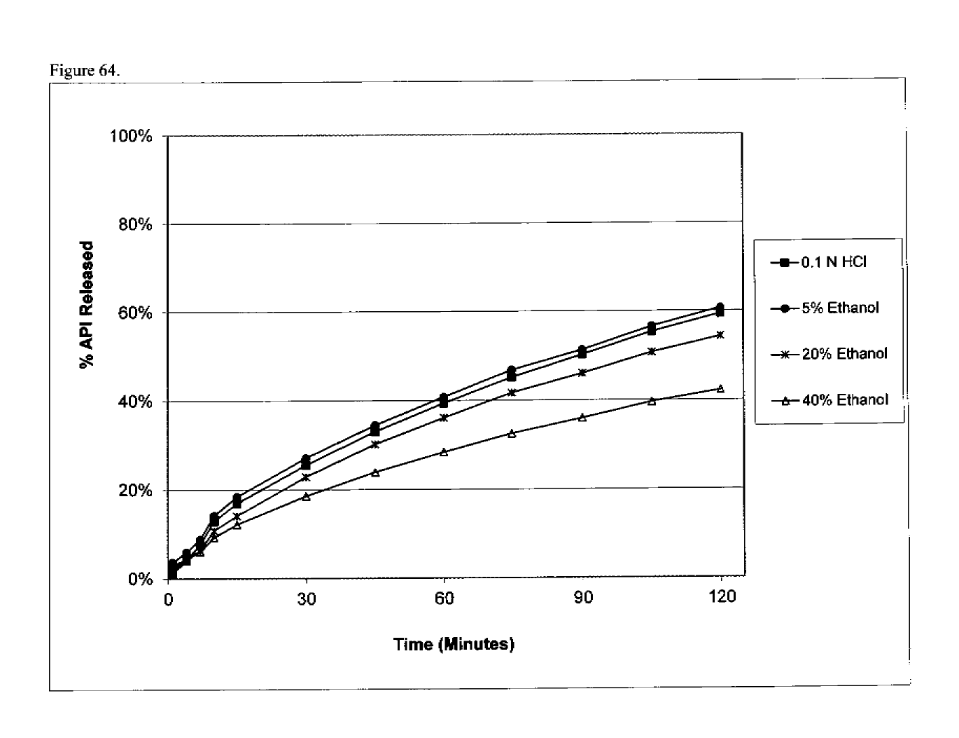

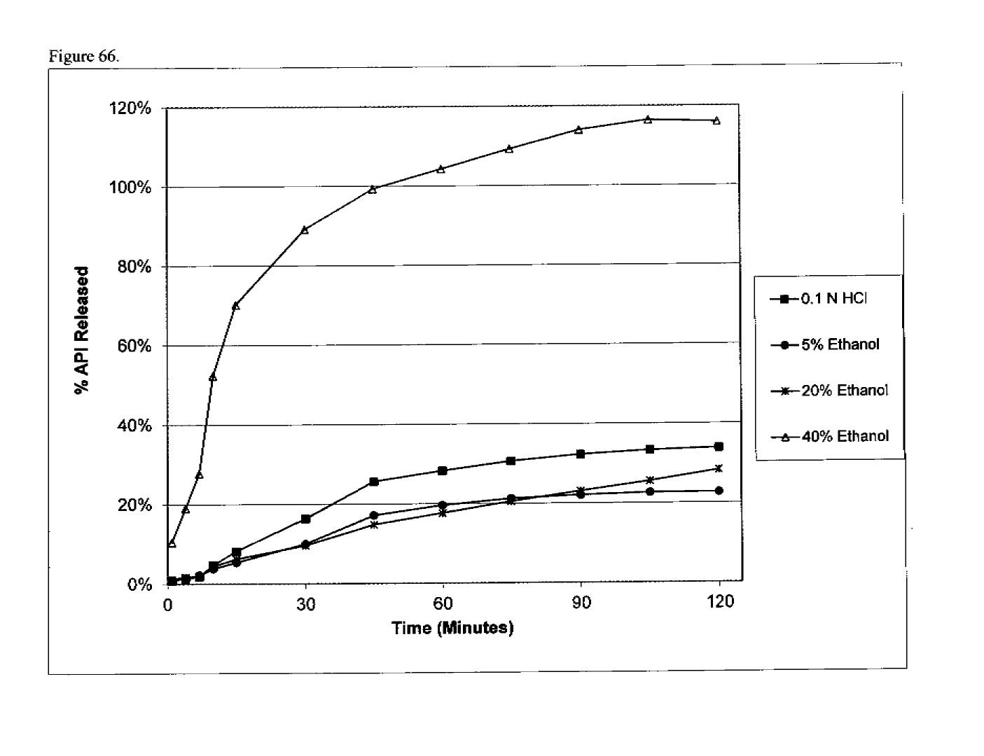

Levorphanol tartrate has a dose dumping profile as summarized in FIG. 60. The tartrate releases at least 60% of the active pharmaceutical after thirty minutes under all ethanol concentrations tested. In contrast, the amorphous levorphanol pamoate's dose dumping profile, FIG. 62, and the polymorphic levorphanol pamoate's dose dumping profile, FIG. 64, while slightly different, still show no propensity for dose dumping. Less than 30% of the active is released after thirty minutes. The two pamoate profiles are modestly different and the minor effect is indicative of their polymorphic differences. FIG. 66 illustrates the dose dumping profile of levorphanol xinafoate, which does not exhibit dose dumping characteristics except for the 40% ethanol condition wherein it could be considered as having immediate release behavior. Clearly, for levorphanol pamoate, no singular advantage can be attributed to a particular amorphous or polymorphic form. However, either of the disclosed pamoates would be far superior at providing an abuse deterrent, extended release levorphanol-based drug product instead of its comparable tartrate salt.

Naltrexone is beneficial for its use in abuse deterrent formulations which also would contain a potentially abused drug substance. Preferably, the naltrexone salt of choice would have formulation and stability compatibility in the dosage presentation, and have physical and chemical properties analogous to abuse deterrent drug substances available as organic acid addition salts, such as pamoate and/or xinafoate salts. In the event an abuse deterrent formulation was devised containing an abused narcotic or opioid and a naltrexone salt, separation of the naltrexone component from the other drug substance is not desired. Hence, formulation compatibility is enhanced, and the potential for separation of naltrexone from the other active is diminished if they have similar chemical and physical properties.

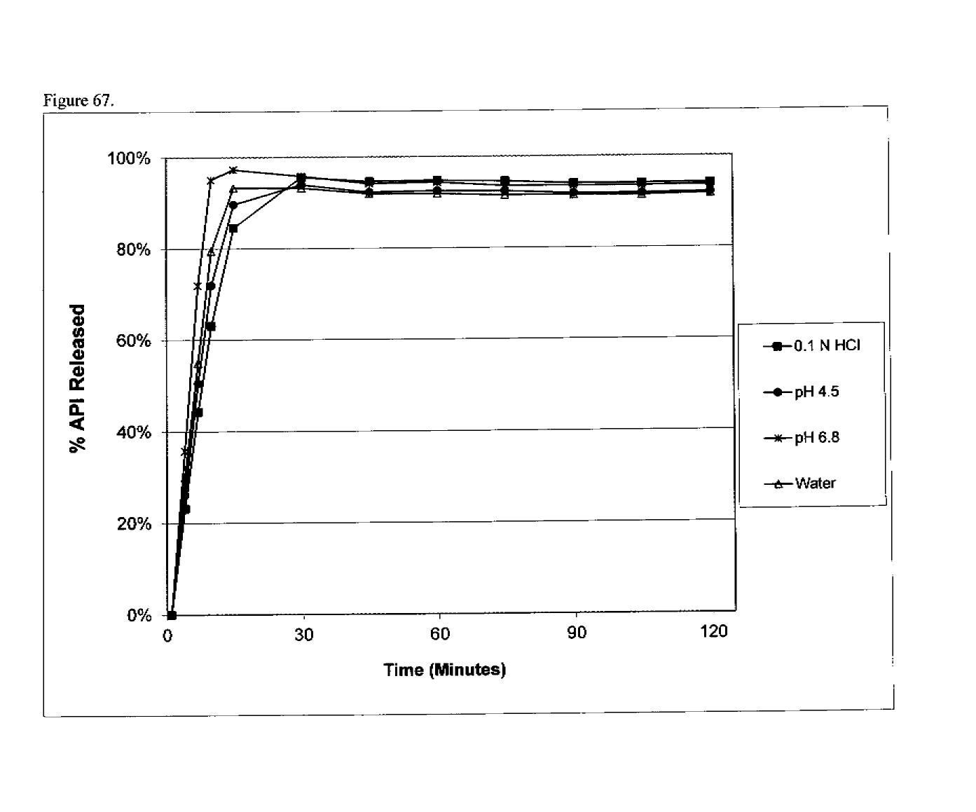

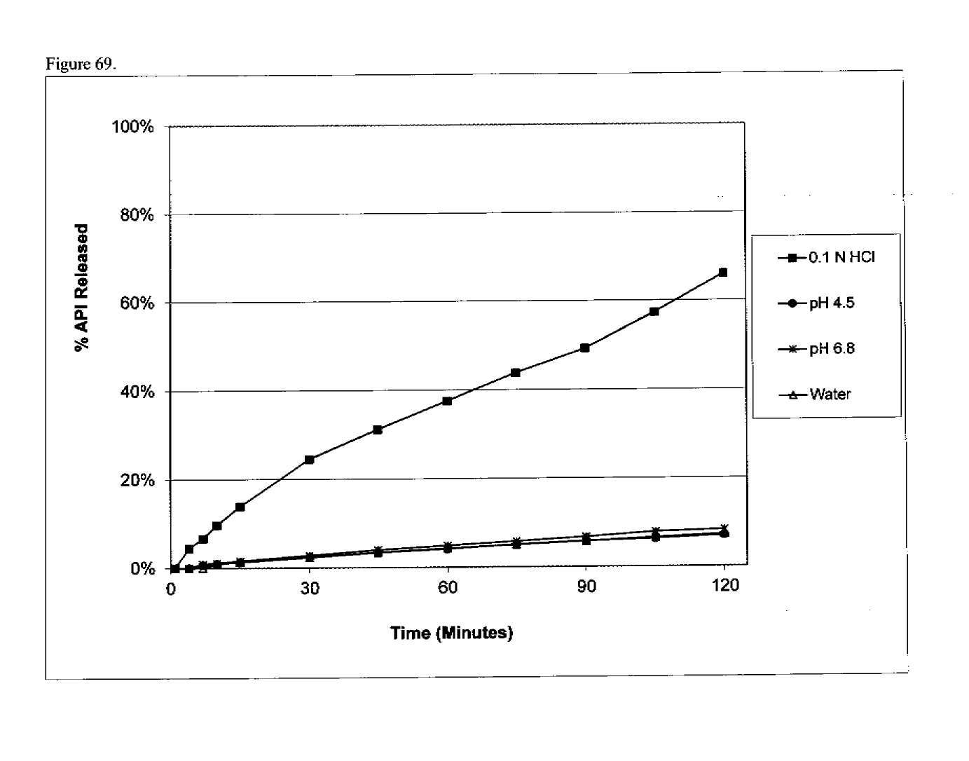

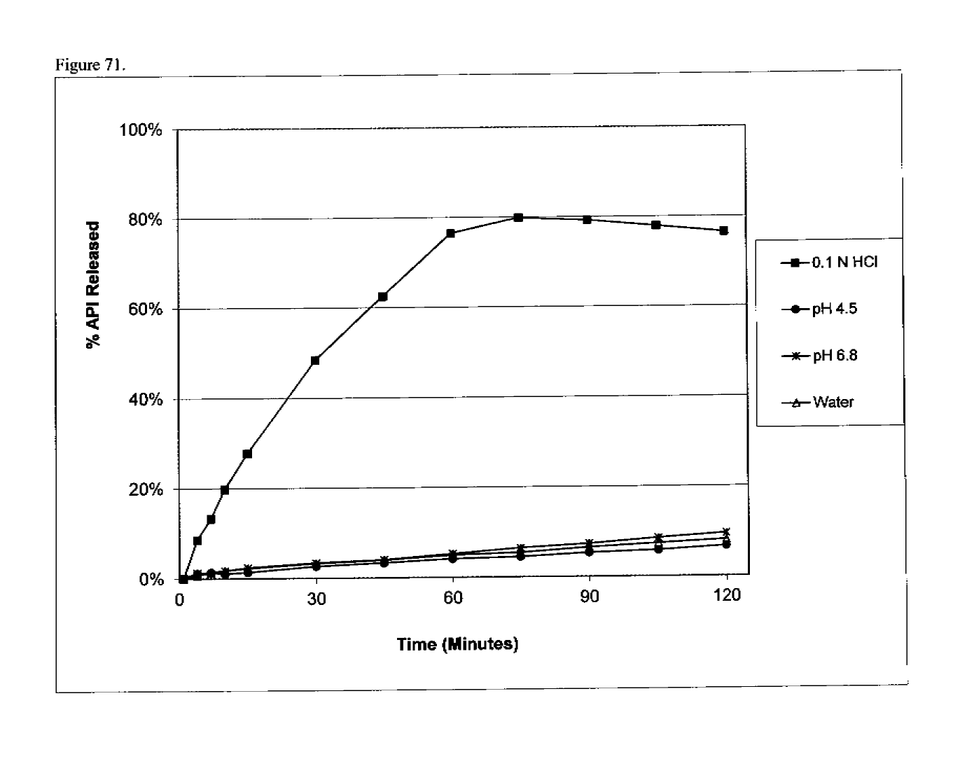

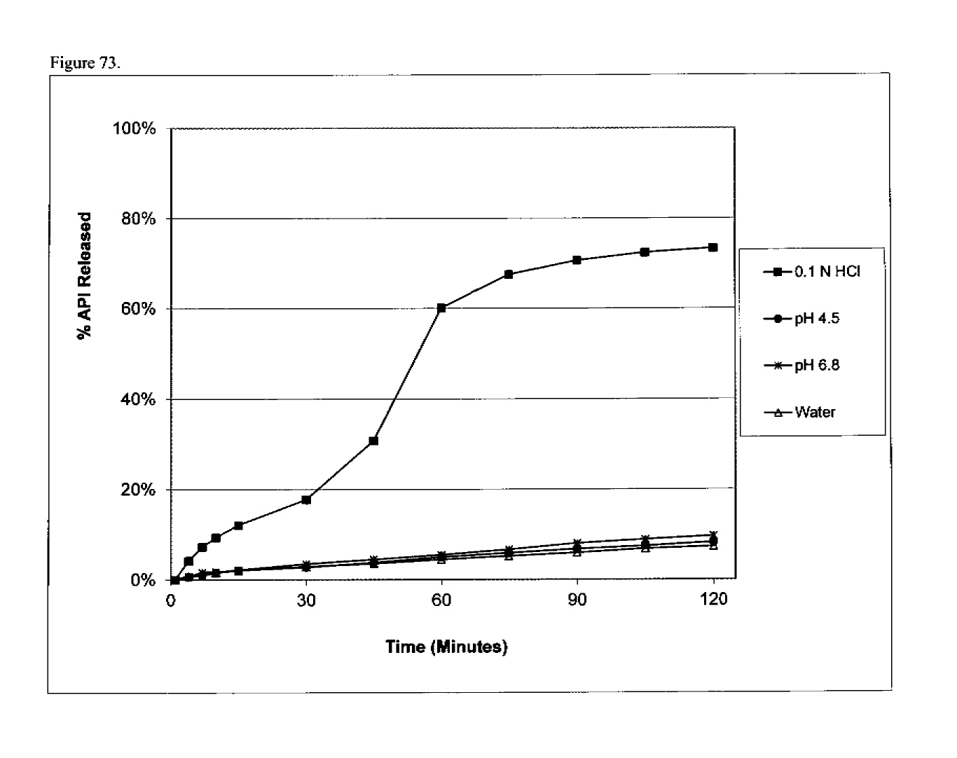

Naltrexone hydrochloride has a pH dependent dissolution profile as illustrated in FIG. 67 and as can be evidenced, the active exhibits immediate release at all pH conditions. In contrast, amorphous naltrexone pamoate, FIG. 69, and polymorphic naltrexone pamoate, FIG. 71, each possess highly attenuated pH dependent dissolution profiles. Some difference is detected between the two pamoates at the pH 1 condition wherein the amorphous form has a release of about 25% at thirty minutes while the polymorphic form at the same time point exhibited a 45% release. Here too, it is unexpected that the polymorphic form would release faster than the amorphous form and this observation is contrary to traditional teaching. Naltrexone xinafoate's pH dependent dissolution profile is strongly attenuated under all pH test conditions; even after thirty minutes there was no appreciable dissolution in 0.1N HCl as illustrated in FIG. 73.

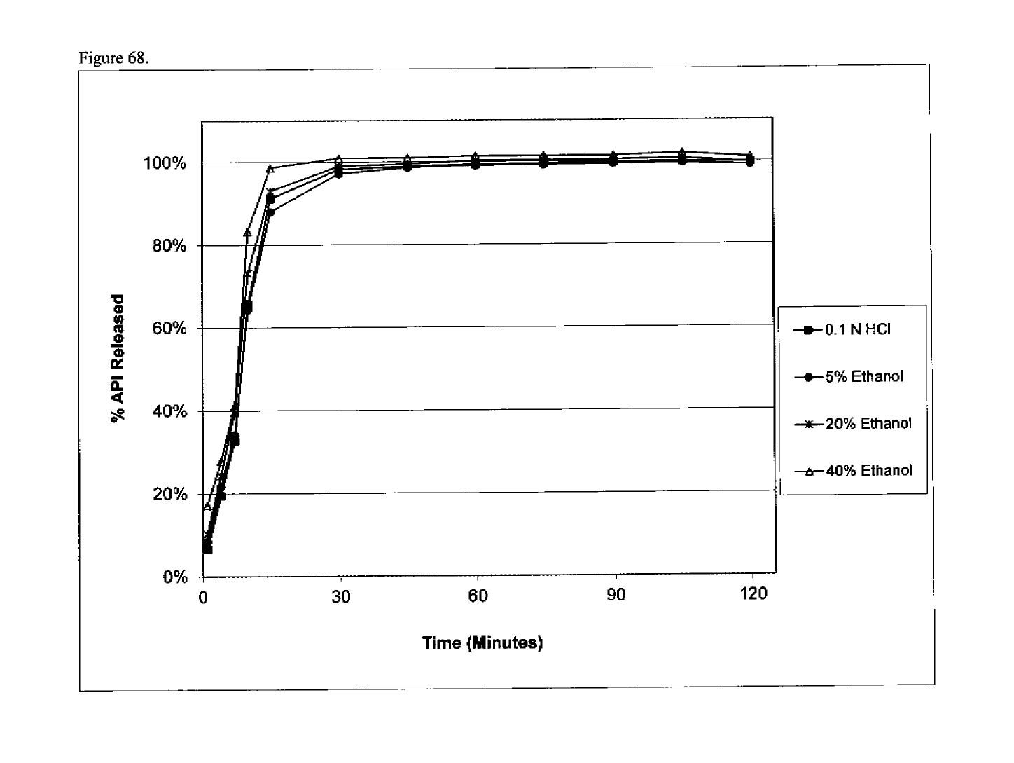

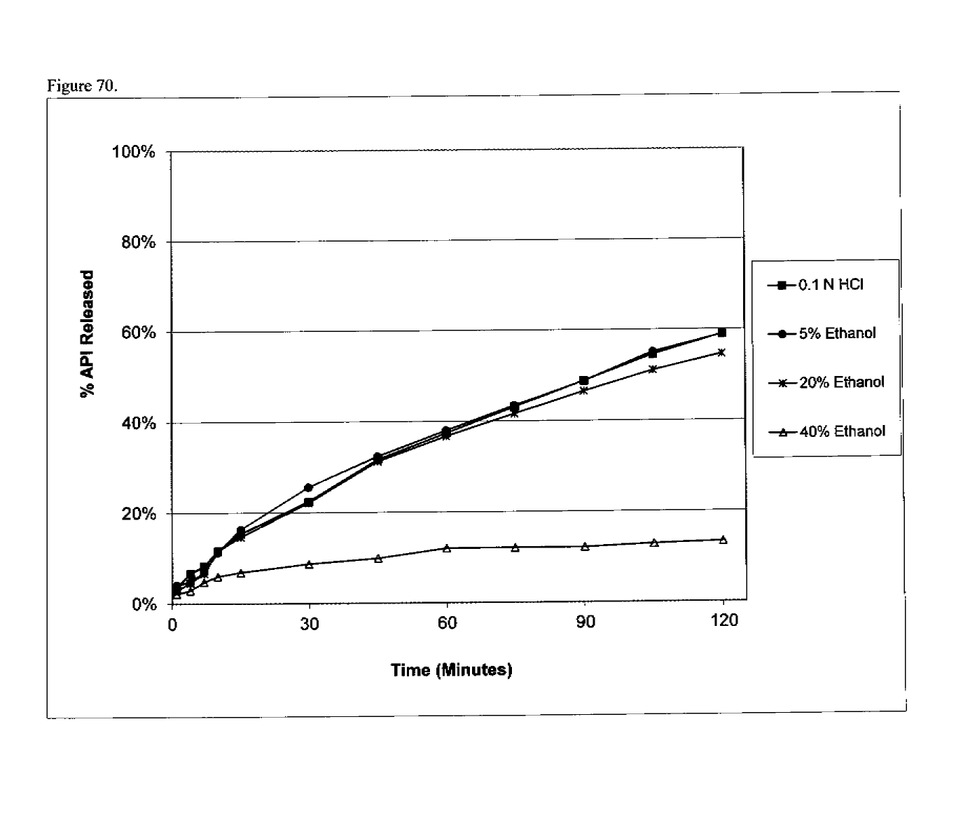

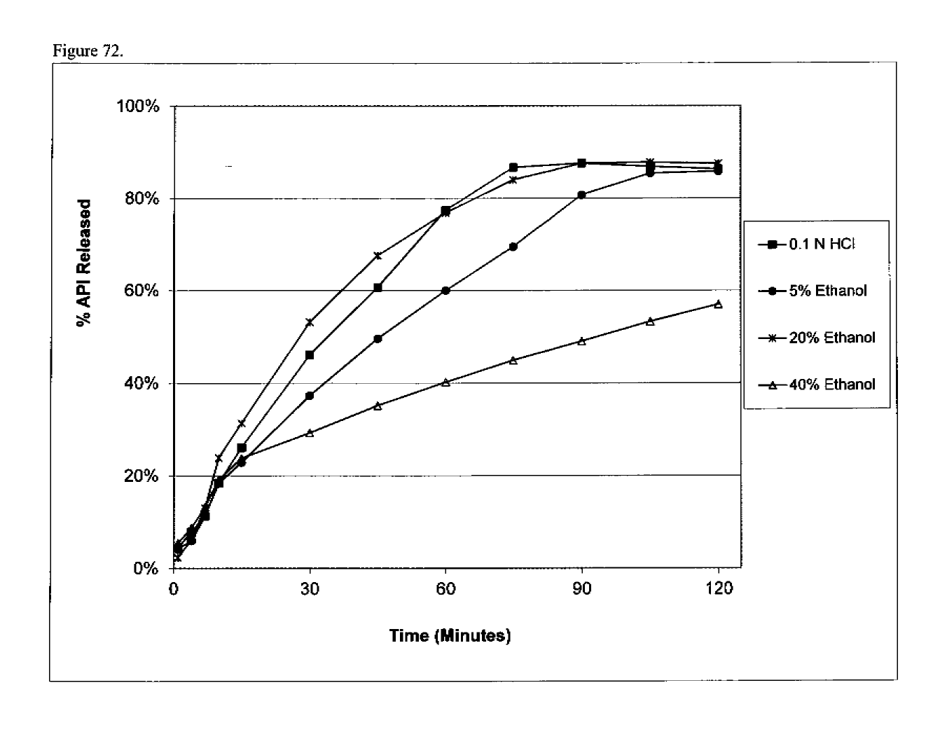

Naltrexone hydrochloride has a dose dumping profile summarized in FIG. 68 and clearly dose dumps under all of the test conditions. In theory, a formulation containing naltrexone hydrochloride, included as a compound to thwart abuse of another medication also in the formulation, could be easily extracted with ethanol to provide the potential abuser with the desired opioid, narcotic, or mind-altering drug substance. In contrast, herein, the discussion will continue to use the terminology of “dose dumping” related to naltrexone for purposes of consistency, but let it be understood that a potential abuser of a combination drug product, such as a formulated dosage containing an abused drug substance and naltrexone, would not be intending to dose dump naltrexone which would inhibit getting “high”. However, the would-be abuser would want to remove naltrexone from the drug product and the ability to do that with ethanol would be desirous for an abuse purpose of the remaining drug product/substance. Amorphous naltrexone pamoate shows no propensity for dose dumping, illustrated in FIG. 70. Polymorphic naltrexone pamoate appears more susceptible to dose dumping, illustrated in FIG. 72, but is still a much better selection than naltrexone hydrochloride if the prevention of dose dumping and/or ethanol extraction is desired. For naltrexone xinafoate, the 40% ethanol condition provides a medium wherein naltrexone is highly dissociated from the xinafoate component at thirty minutes; at the other conditions, naltrexone is only modestly soluble.

An embodiment of the invention is the combination of organic acid addition salts of naltrexone in combination with opioids which are frequently abused. Drug products can be prepared comprising at least one of naltrexone pamoate or naltrexone xinafoate with at least one opioid selected from the group consisting of codeine, hydrocodone, propoxyphene, fentanyl, hydromorphone, levorphanol, meperidine, methadone, morphine, oxycodone, oxymorphone, buprenorphine, butorphanol, nalbuphine, and pentazocine. More particularly, amorphous or polymorphic naltrexone pamoate can be used in a drug product comprising an opioid and an opioid antagonist.

Many, many comparisons such as these are possible from the extensive data set, which may overshadow some fundamental conclusions: the hydrochloride, sulfate and tartrate salts exhibit an immediate release profile and are highly susceptible to dose dumping; the pamoate and xinafoate salts attenuate the pH dissolution profiles of the opioids and impart a level of extended release directly to the active substance; the pamoate and xinafoate salts are suitable for providing pH independent release drug product formulations; the pamoate salts are a dominating factor in preventing dose dumping and are independent of the particular opioid.

An embodiment of the invention is provided by a process for forming a drug substance wherein at least one equivalent of the amine containing drug substance is reacted per mole of disodium pamoate to yield the drug substance pamoic acid salt, preferably in a ratio of 2:1, 1:1, or mixtures thereof. An aqueous acidic solution of the amine containing drug substance is combined with a basic solution of pamoic acid or disodium pamoate. The acid/base reaction ensues and the insoluble organic acid salt precipitates from the aqueous solution. Optionally, the salt can be purified, dried and milled to obtain a drug substance ready for formulation into the desired delivery format. The drug product formulated with the drug substances then possesses the targeted delivery characteristics of the drug substance and the potential for abuse of either the drug substance and/or drug product is eliminated or greatly reduced when abuse is attempted via the mucosal surfaces or by injection.

The organic acid is defined by the following Structures A through G wherein Structure A represents the general family of Markush compounds embodied within the invention. Structure B represents the subset of salicylic acid and its derivatives conceived as a component of this invention. Structures C, D and E are regio-isomeric variations on Compound A wherein two adjacent substituents on Compound A form a fused aryl ring (i.e. R¹+R²; R²+R³; and R³+R⁴). Structures F and G represent a further sub-category of dimer-like compounds derived from Structure A. In Structure F, dimerization has occurred through R⁴ of two Structure A compounds with both possessing fused-aryl ring systems formed via R²+R³. In Structure G, dimerization has again occurred through R⁴ of two Structure A compounds but with both Structure A residues possessing fused-aryl ring systems formed via R¹+R².

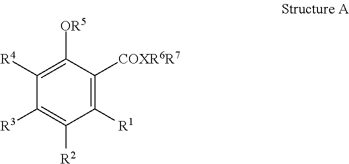

wherein R¹-R⁴ are independently selected from H, alkyl or substituted alkyl of 1-6 carbons, adjacent groups may be taken together to form a cyclic alkyl or cyclic aryl moiety; R⁵ represents H, alkyl, alkylacyl or arylacyl; R⁶ and R⁷ are independently selected from H, alkyl of 1-6 carbons, aryl of 6-12 carbons, alkylacyl or arylacyl analogues sufficient to satisfy the valence of X (e.g. to provide a mixed anhydride or carbamate); X is selected from nitrogen, oxygen or sulfur, and when X═O, R⁶+R⁷ may represent an alkali earth cation, ammonium or together form a heterocyclic moiety.

Particularly preferred organic acids include Structures B through E.

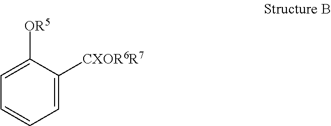

wherein R⁵, R⁶, R⁷ and X remain as defined above for Structure A;

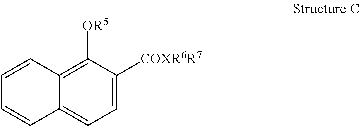

wherein X, R⁵, R⁶ and R⁷ remain as defined above for Structure A and more preferably X is O;

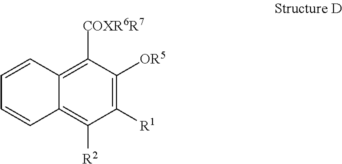

wherein X, R¹, R², R⁵, R⁶ and R⁷ remain as defined above for Structure A and more preferably X is O; R¹ and R² are hydrogen;

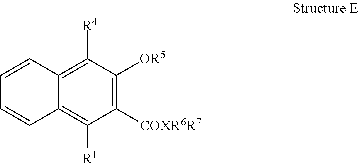

wherein X, R¹, R⁴, R⁵, R⁶ and R⁷ remain as defined above for Structure A and more preferably X is O, R¹ and R⁴ are hydrogen;

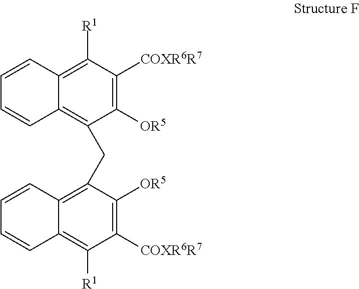

wherein X, R¹, R⁵, R⁶ and R⁷ are independently defined as above for Structure A and more preferably at least one X is O and at least one R¹ is hydrogen; and

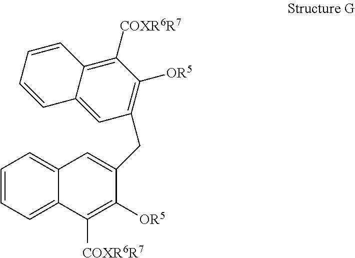

wherein X, R⁵, R⁶ and R⁷ are independently defined as above for Structure A and more preferably X is O and R⁵ is hydrogen.

Pamoic acid, or a synthetic equivalent of pamoic acid, is the preferred embodiment. Pamoic acid has a formula corresponding to Structure F wherein X is O; R¹ is independently selected from hydrogen, alkyl or substituted alkyl of 1-6 carbon atoms, and R⁵, R⁶ and R⁷ are hydrogen.

A synthetic equivalent of pamoic acid is a material that provides the structural moiety independent of its particular salt, ester, or amide form and that upon pH adjustment yields pamoate functionality suitable for reaction, optionally with one or two equivalents of an amine-containing active pharmaceutical ingredient to form a pamoate salt. Examples of synthetic equivalents of pamoic acid capable of manipulation to produce pamoate salts include but are not limited to, disodium pamoate, mono-alkali pamoate, di-ammonium pamoate, di-potassium pamoate, lower molecular weight di-alkyl and/or di-aryl amine pamoate, lower molecular weight di-alkyl and/or di-aryl esters of pamoic acid, and lower molecular weight di-alkylacyl and/or di-arylacyl O-esters of pamoic acid, i.e. those alkylacyl and arylacyl esters formed using the hydroxyl moiety of pamoic acid and not the carboxylic acid functional group. The descriptor phrase “lower molecular weight” used above means the indicated moiety has a molecular mass contribution within the pamoate derivative of less than about 200 amu.

In an embodiment an drug substance can be formulated in a drug product with a compound of Formula H which renders the drug substance less susceptible to dose dumping:

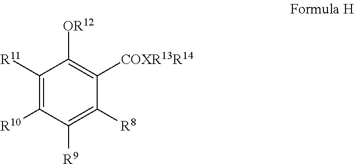

wherein R⁸-R¹¹ are independently selected from H, alkyl or substituted alkyl of 1-6 carbons, adjacent groups may be taken together to form a cyclic alkyl, cyclic alkyl-aryl, or cyclic aryl moiety;
R¹² is selected from H, or an alkali earth cation;
R¹³ and R¹⁴ are independently selected from H, alkyl of 1-6 carbons, an alkali earth cation, and aryl of 6 to 12 carbons, in a number sufficient to complete the valence bonding of X, and
wherein X is selected from nitrogen, oxygen or sulfur.

Particularly preferred examples of compounds of Formula H include 3-hydroxy-2-naphthoic acid (Bon Acid), disodium pamoate (OTHERS??)

For clarity, the use of lower molecular weight di-alkyl or di-aryl amine pamoate allows for the exchange of higher molecular weight amines, or drug free bases, to be exchanged for the lower molecular weight amine component during the salt formation reaction. Similarly, the use of lower molecular weight di-alkylacyl and/or di-arylacyl pamoates allow for their conversion through ester hydrolysis to the pamoic/pamoate moiety followed by reaction with the desired drug free base.

A particularly preferred embodiment and method of administering the amine-containing pharmaceutically active compound is by oral dose. The oral dose is prepared by first preparing an organic acid salt of the active compound. The organic salt is then formulated into a carrier matrix to provide an oral dose drug product. The carrier matrix is composed of ingredients, or excipients, optionally selected from the group, but not limited to binders, fillers, flow enhancers, surfactants, disintegrants, buffers, and the like, typically employed in the art and found in the “Handbook of Pharmaceutical Excipients”, Rowe, Sheskey and Owen (Editors), Fifth Edition, 2006, Pharmaceutical Press (publishers). When the oral dose is ingested the organic salt dissociates under physiological conditions. The organic acid portion of the amine-containing organic acid addition salt forms the insoluble organic acid while the active compound is liberated and becomes bio-available. Efforts to directly isolate the active compound from the oral dose would be thwarted as described herein.

The manufacture of a formulated drug product optionally includes, but is not limited to the following steps: wet or dry granulation; direct compression tablet pressing particle coating followed by drying; sieving and/or sizing milling; blending with additional excipients; optionally, additional wet or dry granulation; optionally, sizing and milling blending with additional excipients; tablet pressing or capsule filling; pan or tumbler coating and drying; and packaging.

The drug products of the instant invention can be formulated as a tablet, a capsule, a caplet, a suspension or an injectable. The present invention is applicable to a variety of drug delivery presentations including solid oral dose, parenteral dosage forms (depo-type products) and by devices and formulations suitable for transdermal delivery and inhalation administration. It is responsibly acknowledged that many factors may influence the overall pharmacokinetic profile of a drug product. For instance, the particle size distribution of the drug substance may markedly influence drug substance bioavailability. Hence, the optimum practice of this invention when employed for a specific drug product must account for the multitude of additional factors. The benefit of the current invention is a means to provide a dominating or controlling factor to prevent abuse while achieving efficacious and therapeutic patient dosages to which refinements, adjustments or modifications can be asserted to yield an optimal response.

In the present invention a drug product can be prescribed and administered in a manner wherein proper administration provides a therapeutic effect and the function of the API is realized. With a different manner of administration, in other words, a non-therapeutic administration of the drug product, the API does not enter the bloodstream in an amount sufficient to be active. To be effective the API must be bio-available. For the purposes of the present invention, one method of establishing a compound's bio-availability is by determining the percentage of weight API recovered from an aqueous solution at a pH representative of the method of administration described herein. For the purposes of the present invention a compound is considered to be effective when less than 85 wt % of the compound is recovered from an aqueous solution at a pH representative of the method of administration. If, by contrast for example, 85 weight percent or more of a drug compound is recovered from a solution at a pH of 4-9, pH 7 for example, the material is considered to be bio-unavailable at a mucosal membrane and is considered non-permeable at the mucosal membrane and the compound exhibits prophylactic properties. If, for example, less than 85 weight percent of a drug compound is recovered from a solution at a pH of less than 4, pH 1 for example, the material is considered to be bio-available under oral administration, for gastrointestinal tract bioavailability, and is considered permeable in, for example, the gastrointestinal tract due to the release of the API at the pH of the gastrointestinal tract. For the purposes of the present invention therapeutic dose is characterized as immediate dose, slow dose and controlled dose. An immediate dose is defined as a formulation wherein at least 85 wt % of the active ingredient is bioavailable at 1 hour at a representative pH, as for example, 0.1 N HCl. For the purposes of the present invention bioavailable is defined as the weight percent which is not recovered by filtration. Slow release is defined as a formulation wherein at least 50 wt % to less than 85 wt % of the active ingredient is bioavailable at 1 hour at a representative pH. Controlled release is defined as a formulation wherein no more than 50 wt % of the active ingredient is bioavailable at 1 hour at a representative pH. More preferably, with controlled, or extended, release at least 12.5 wt % to no more than 42.5 wt % is bioavailable at 1 hour at a representative pH. In one embodiment the representative pH approximates the stomach pH which corresponds to 0.1 N HCl. It is particularly preferred that the representative pH be between 1.6 and 7.2.

Enteric coatings are well known in the art to inhibit release of a drug substance from a drug product at low pH, such as the pH of the stomach, thereby allowing the drug substance to pass into the intestine, which have a higher pH, before the drug substance can be released from the drug product. Enteric coatings can include various materials, typically polymeric materials, with cellulose based materials being exemplary. Without limit thereto materials suitable for demonstration of the invention include methyl or ethyl acrylate-methacrylic acid copolymers, cellulose acetate succinate, cellulose acetate phthalates, hydroxyl propyl methyl cellulose phthalate, polyvinyl acetate phthalate, methyl methacrylate-methacrylate acid copolymers, shellac, cellulose acetate trimellitate, sodium aginate and copolymers or mixtures.

An embodiment of the invention is represented by oxymorphone xinafoate (FIG. 51) and codeine xinafoate (FIG. 57) both of which do not release their respective active ingredient, oxymorphone or codeine, in 0.1 N HCl which is representative of stomach pH. However at higher pH, representative of colon or intestinal pH, the active ingredient is released as realized in FIGS. 51 and 57. These drug substance therefore allow for the formulation of a drug product which passes through the stomach for release in the colon or intestines without the necessity of an enteric coating. This is a clear advance in the art

Throughout the specification the term organic acid is used generically to refer to the acid form or the salt form of a compound.

From the discussion above, the pamoate and xinafoate drug substance salts can be easily differentiated from the currently abused drug substances and would satisfy the requirements for Tiers 1 through 4 label claims. The unusual properties and characteristics of pamoate and xinafoate drug substance salts clearly establish their effectiveness at providing physical and chemical barriers to abuse; these properties would extend to products formulated with these salts and the product could include additional physicochemical barriers. With these barriers established at the drug substance level, manipulation for purposes of abuse is thwarted and statistically meaningful pharmacovigilance results would indicate lower or reduced abuse rates.

It has been demonstrated that organic acid addition salts of pharmaceutically active amines provide a route to achieving abuse deterrent design features at the drug substance level. Of specific interest herein are the pamoate and xinafoate salts of oxymorphone, codeine, levorphanol and naltrexone. The analytical characterization of the organic acid addition salts of amine containing opioids, narcotics and otherwise pain relieving medications, or mind altering active ingredients, encompasses structural elucidation, thermal characterization, amorphous or polymorphic determination and dissolution properties. Structural elucidation is accomplished by proton nuclear magnetic resonance spectroscopy (¹H NMR), Fourier transform infrared spectroscopy (FTIR), and high pressure liquid chromatography (HPLC) comparison to known standards. The amorphous or polymorphic composition of the compounds is determined principally, and jointly, by differential scanning calorimetry (DSC) and powder X-ray diffraction (PXRD). Finally, the dissolution properties of the compounds, and any (poly)morphic differentiation they exhibit toward dissolution media are evaluated. Both the pH dependent, and acidic, ethanol induced dose dumping dissolution profiles are established and the results represent an analytical characterization of how different polymorphs of the otherwise identical compounds respond to different dissolution conditions. In this manner, the physical and chemical characterization of a compound, and its (poly)morphic form, are connected to the compound's predicted response when included in an orally administered drug product.

For a given salt, the particular amorphous or polymorphic form, as uniquely characterized herein, provides another discriminating factor for selecting an abuse deterrent form of the abuse deterrent salt since significant dissolution profile differences can be observed for otherwise identical salts. The experimental contribution to successfully identifying an abuse deterrent salt and salt form should not be underestimated. Thus, the traditional expectations long established in pharmaceutical science do not always prevail. For instance, the xinafoate salts are often highly crystalline materials; however, of the xinafoate salts disclosed herein, only naltrexone xinafoate exhibits crystalline characteristics by PXRD. In another instance, the polymorphic forms of pamoate salts have significantly faster dissolution profiles in 0.1 N HCl than the corresponding amorphous form, such as oxymorphone pamoate and naltrexone pamoate. Traditionally, the physical state of a material could be quickly screened by DSC such that a broad phase transition temperature usually was consistent with an amorphous bulk structure and a sharp phase transition corresponded to the sample possessing polymorphic/crystalline characteristics. However, for the pamoate and xinafoate salt exemplars disclosed, PXRD appears to be more conclusive than DSC alone. Indeed, full analytical characterization by experimental means requires HPLC analysis, with appropriate standards, to demonstrate the stoichiometric relationship of salt formation with corroboration by ¹H NMR, and FTIR confirming salt formation and not a mere physical mixture. DSC and PXRD contribute to identifying the form of the salt to distinguish amorphous, polymorphic or a mixture. The salt forms are further differentiated by their rate of release in various dissolution media, such as the rate of release as a function of pH dependent and ethanol concentration media. The unexpected properties associated with pamoate and xinafoate salts add significant complexity to API and drug product development well beyond the problem usually trying to be solved—that is, to deliver a drug substance with therapeutic value to a patient in medical need. The problem in the context of a drug abuse epidemic is to deliver a drug substance with therapeutic value to a patient in medical need, but only in a fashion in which the product has abuse deterrent features. By any practical evaluation which acknowledges the epidemic, the “standard” drug substance salts do not possess the requisite differentiating characteristics which can be found with pamoate and xinafoate salts of abuse potential active ingredients.

### EXPERIMENTAL METHODS

### Differential Scanning Calorimetry

Samples were evaluated using a Differential Scanning calorimeter from TA Instruments (DSC 2010). Prior to analysis of samples, a single-point calibration of the TA Instruments DSC 2010 Differential Scanning calorimeter (DSC 2010) with the element indium as calibration standard (156.6±0.25° C.) was completed.

### Infrared Spectroscopy

IR Spectra were obtained in a KBr disc using a Perkin Elmer Spectrum BX Fourier Transform Infrared Spectrophotometer.

### Powder X-Ray Diffraction (PXRD)

Powder X-ray diffraction patterns were acquired on a Scintag XDS2000 powder diffractometer using a copper source and a germanium detector. A powder is defined herein as amorphous if the counts per second of the underlying broad (>2° 2θ at half height) absorption exceeds the counts per second of narrow (<5° 2θ at half height) peaks rising there above. A powder is defined herein as crystalline if the counts per second of the underlying broad (>20° 2θ at half height) absorption is less than the counts per second of narrow (<5° 2θ at half height) peaks rising there above. Crystalline and polycrystalline are not distinguished herein. Crystalline materials are defined as having a morphology even if the actual morphology is not elucidated. Polycrystalline materials are defined as being polymorphic.

### High Pressure Liquid Chromatography (HPLC)

HPLC analyses were performed on a Waters 2695 HPLC system equipped with a Waters 2996 photo diode array detector.

### ¹H NMR Spectroscopy

¹H NMR spectra were obtained on a 400 MHz Varian Inova 400 spectrometer. Spectra were referenced to solvent (DMSO-d₆).

### Dissolution

Dissolution testing was performed using a Distek Dissolution System 2100 consisting of six 1000 mL dissolution vessels with covers containing sampling ports, six stainless steel paddles and spindles, RPM control unit, and a Distek TCS0200C Water Bath, Temperature Controller Unit.

### Examples

### Example 1

### Preparation of Amorphous Oxymorphone Pamoate (2:1)

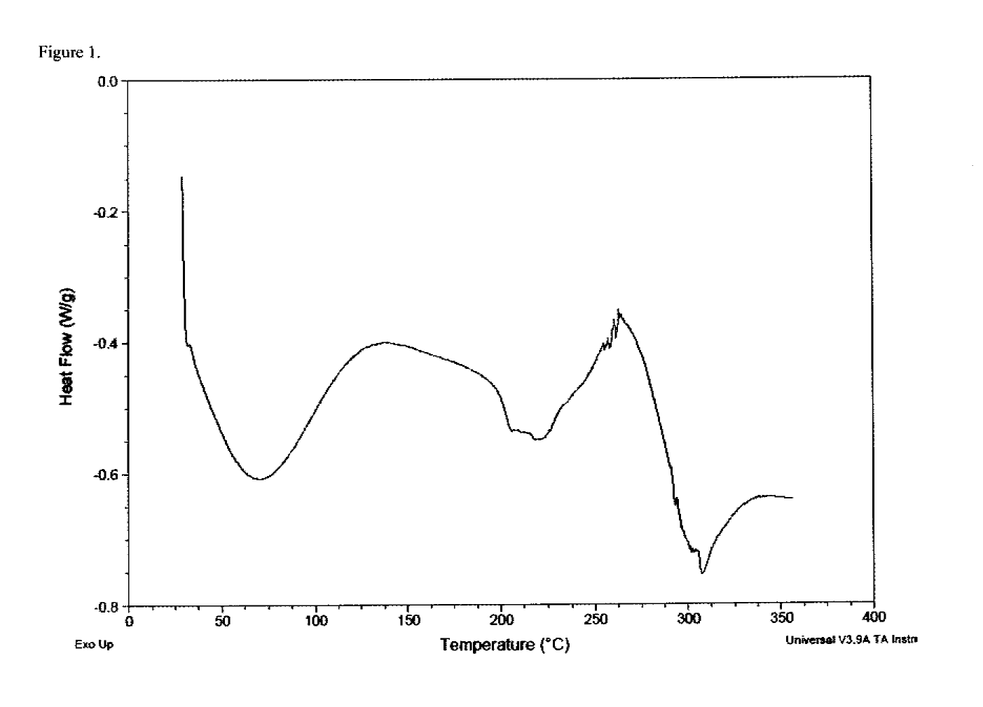

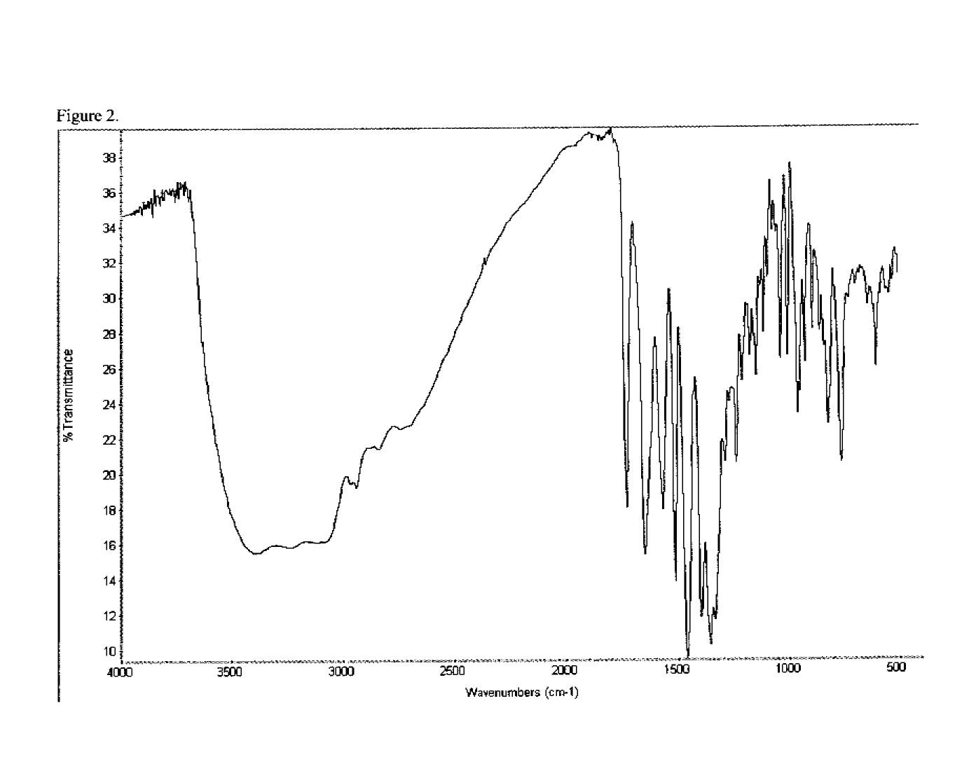

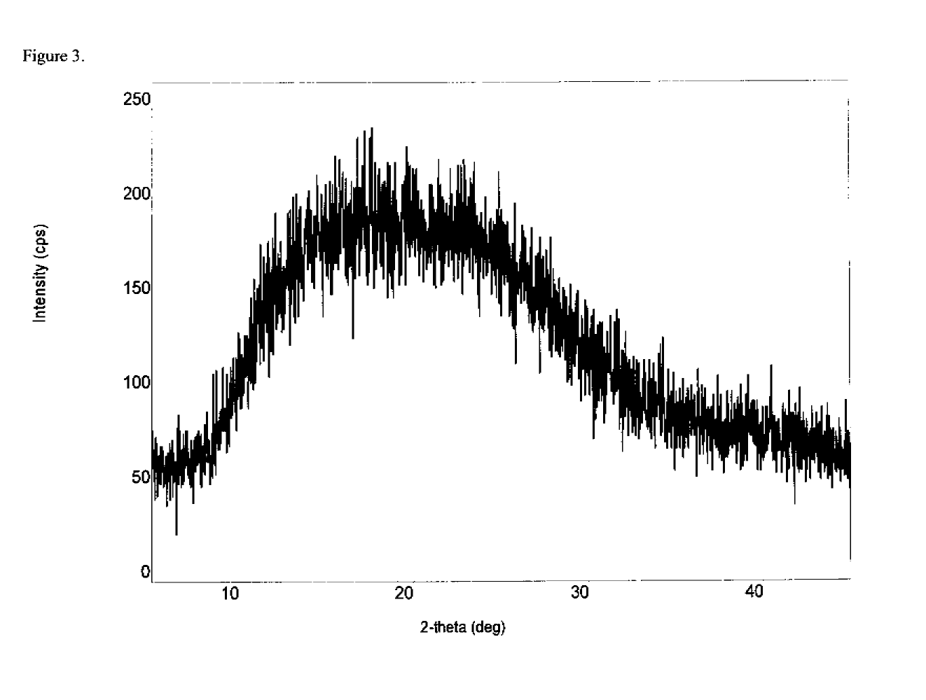

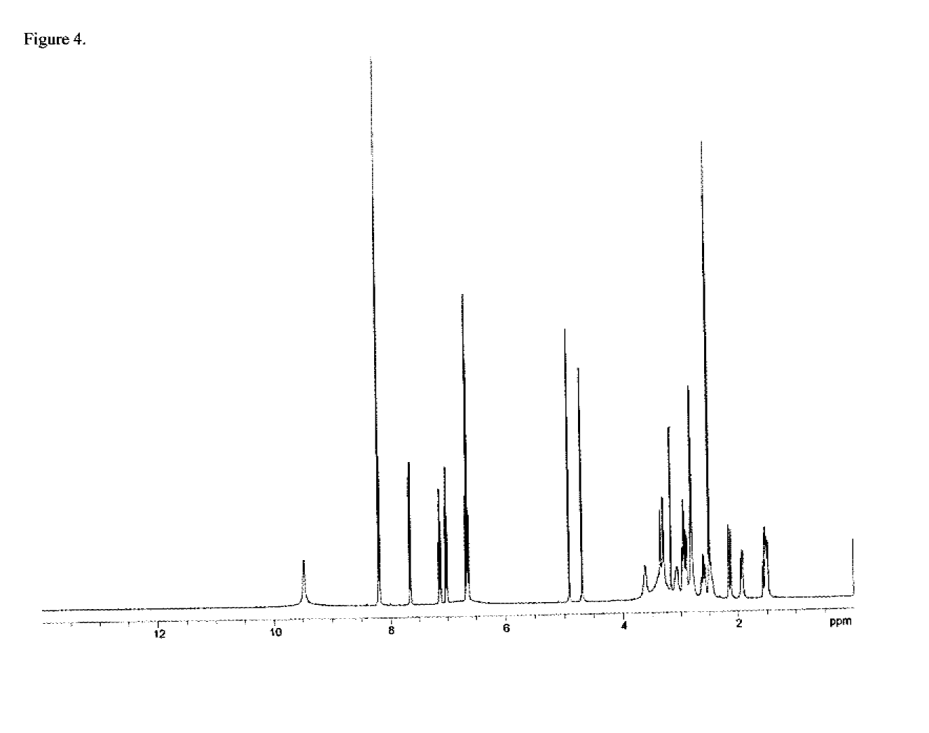

To a 250 mL 3-neck round-bottom flask equipped with a mechanical stirrer, thermowell, nitrogen inlet and addition funnel was charged disodium pamoate (1.90 g, 4.22 mmol) and water (54 g). The pH was adjusted to about 9 with 1 drop of 1N sodium hydroxide (90 mg) and stirred under nitrogen. A solution of oxymorphone hydrochloride (3.0 g, 8.88 mmol) in water (33 g) was prepared by charging to a 100 mL beaker and stirring with a magnetic stir bar. The oxymorphone hydrochloride solution was added dropwise to the disodium pamoate solution via addition funnel over 5 minutes. The resulting off-white slurry was stirred for 5 hours at ambient temperature followed by the solids collected by filtration through a medium fritted filter and thereupon washed with four 50 mL portions of water. The product was dried overnight under reduced pressure to provide 3.59 g (86% yield) of an off-white solid (Karl Fischer, or KF, 1.8% H₂O) which was analyzed by DSC (FIG. 1), FTIR (FIG. 2), PXRD (FIG. 3) and ¹H-NMR (FIG. 4). The ¹H-NMR spectrum was consistent with a compound having a 2:1 ratio of oxymorphone to pamoate and the PXRD diffractogram indicated the salt was amorphous.

### Example 2

### Preparation of Polymorphic Oxymorphone Pamoate (2:1)

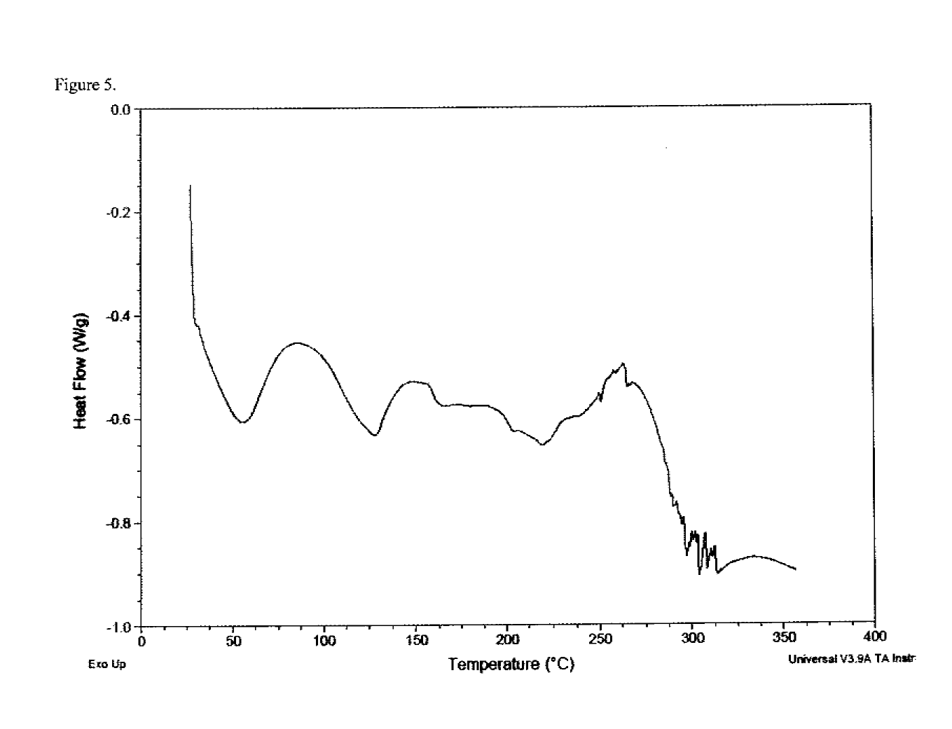

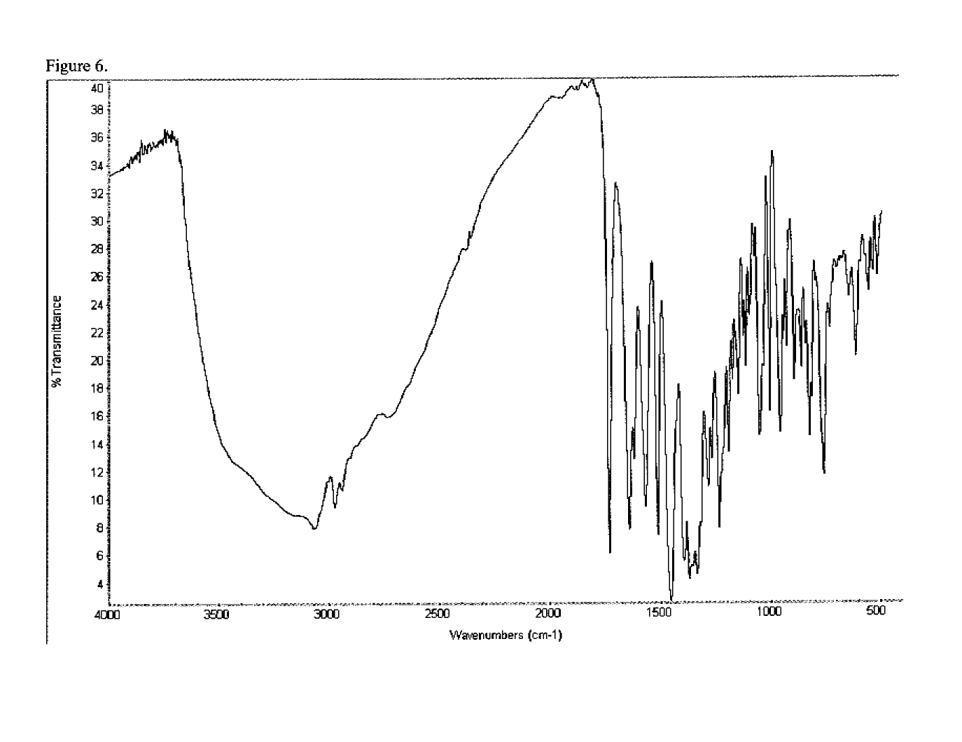

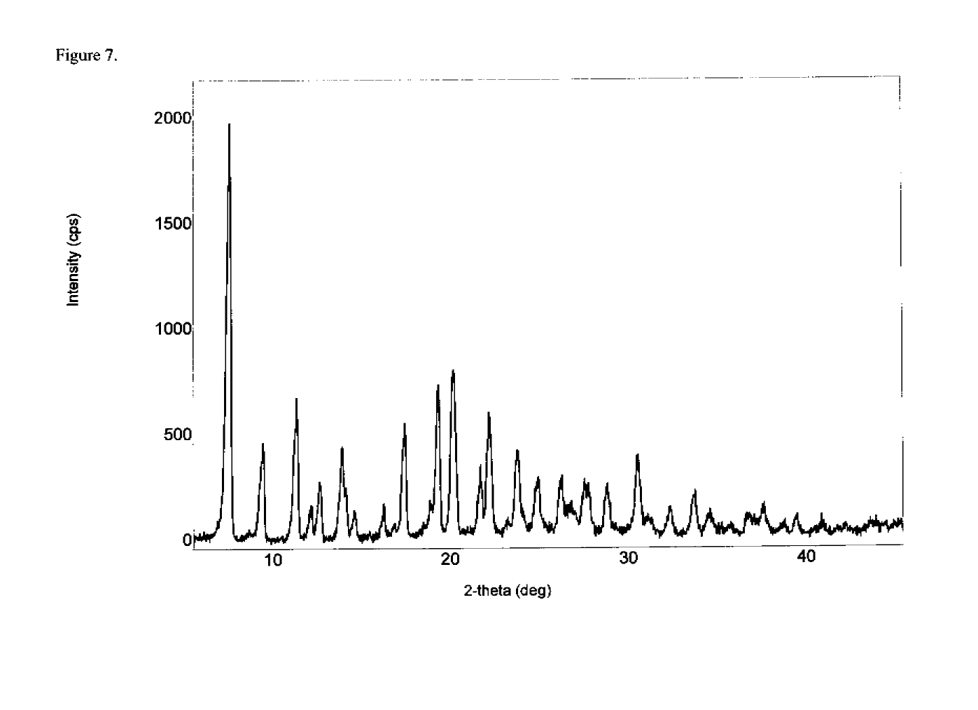

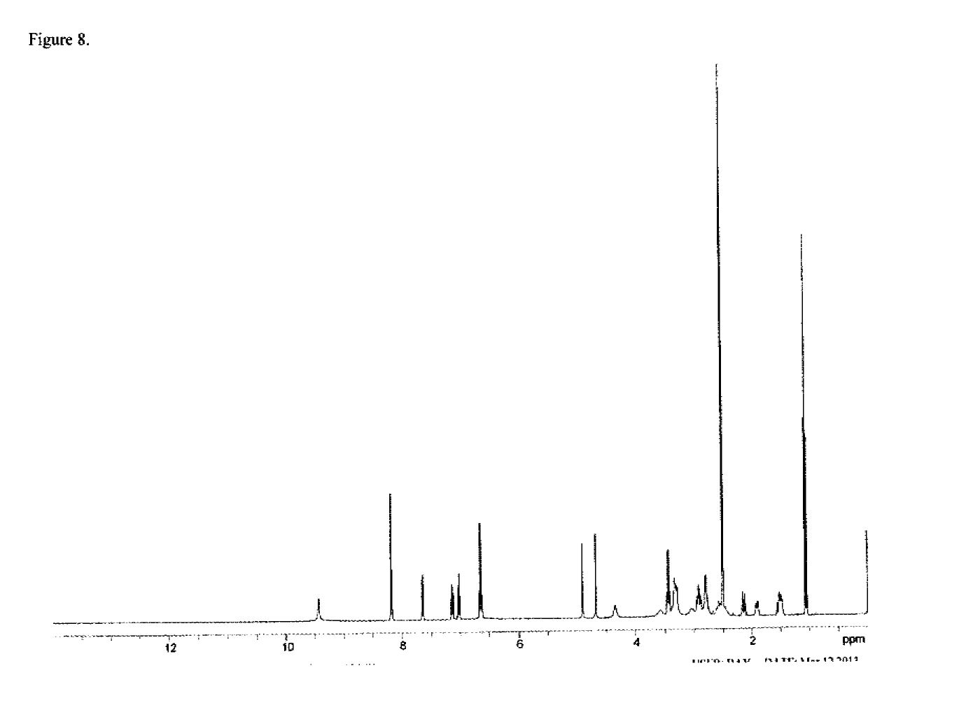

To a 100 mL round-bottom flask equipped with a magnetic stir bar, thermowell and nitrogen inlet was charged amorphous oxymorphone pamoate (1.0 g, 1.01 mmol) and a 98:2 ethanol-water solution (43.5 g). The combined solution was heated to 76° C. under nitrogen for 5-6 hours and subsequently cooled in an ice bath to about 10° C. The solids were collected by filtration through a medium fritted filter and washed with a very small portion of ethanol. The product was dried overnight under reduced pressure to provide 0.87 g (87% yield) of an off-white solid (KF 7.41% H₂O) which was characterized by DSC (FIG. 5), FTIR (FIG. 6), PXRD (FIG. 7), and ¹H-NMR (FIG. 8). The ¹H-NMR spectrum was consistent with a compound having a 2:1 ratio of oxymorphone to pamoate and the PXRD diffractogram indicated the salt was crystalline.

### Example 3

### Preparation of Oxymorphone Xinafoate

To a 100 mL three-neck round bottom flask equipped with a mechanical stirrer, reflux condenser, thermowell and nitrogen inlet was charged BON (beta-oxy-naphthoic acid, also known as 2-hydroxyl-3-carboxy naphthalene (1.63 g, 8.67 mmol) and water (23 g). Sodium hydroxide (ACS grade, 346.8 mg, 8.67 mmol) was added and the solution heated to 50° C. under nitrogen until all the solids dissolved. The sodium xinafoate solution was then cooled to ambient temperature.

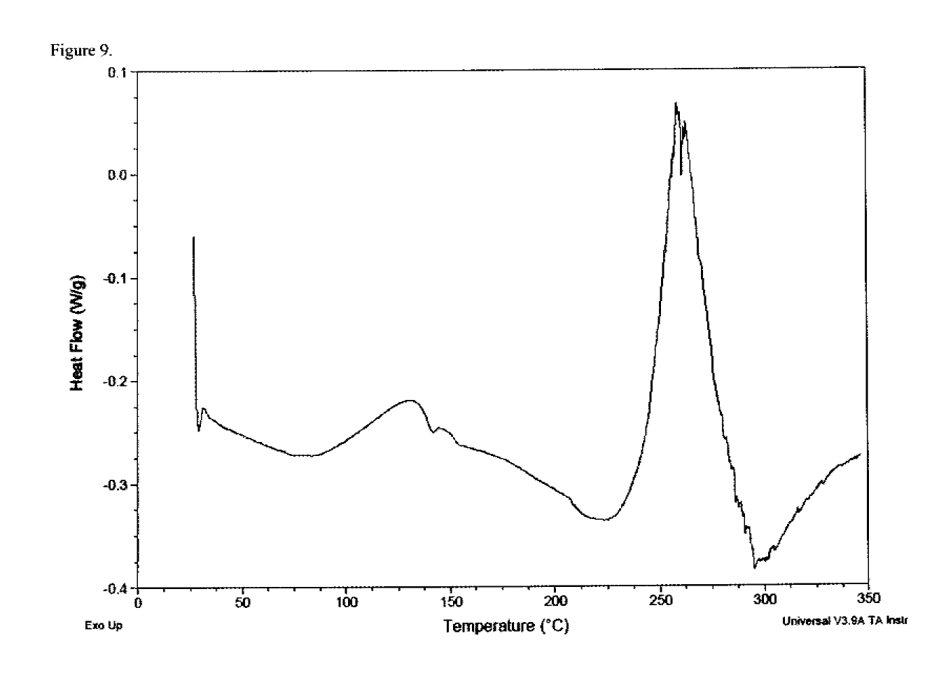

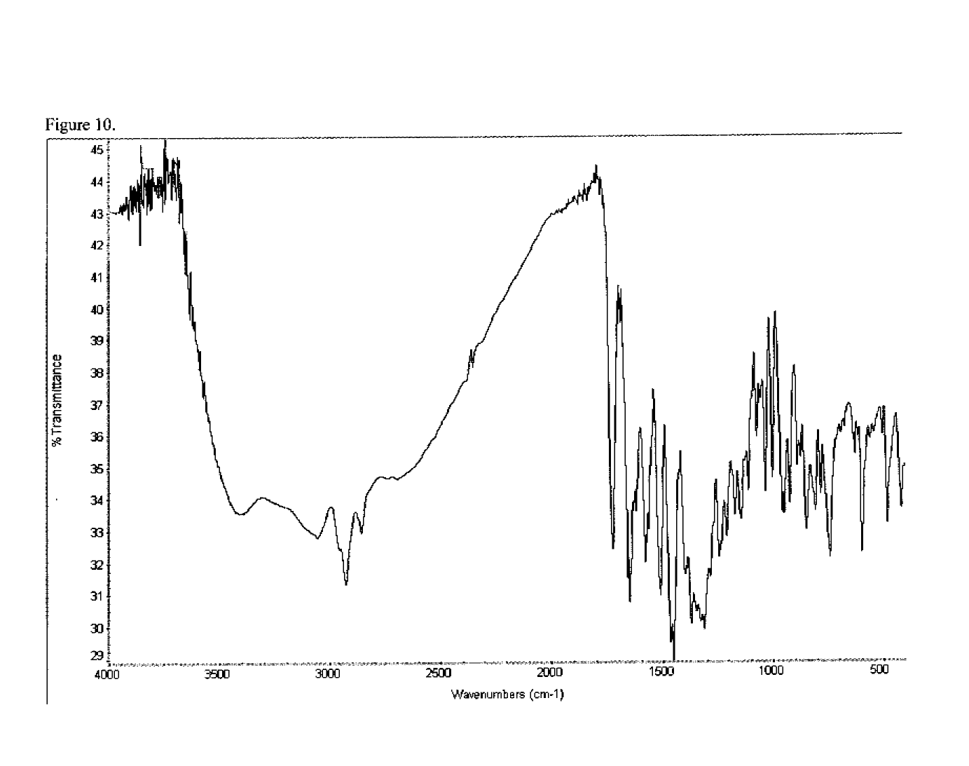

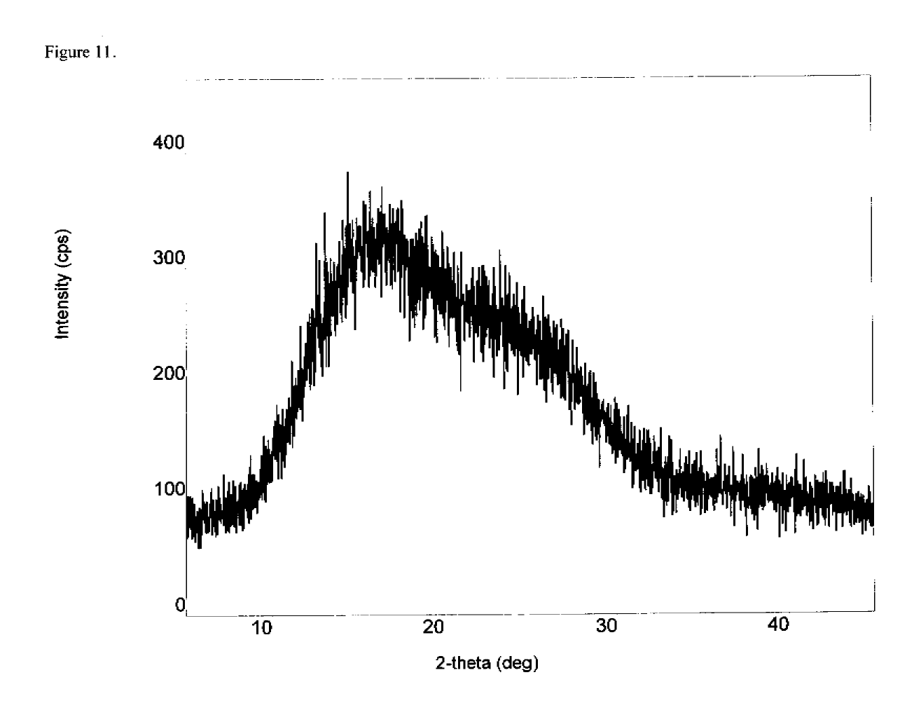

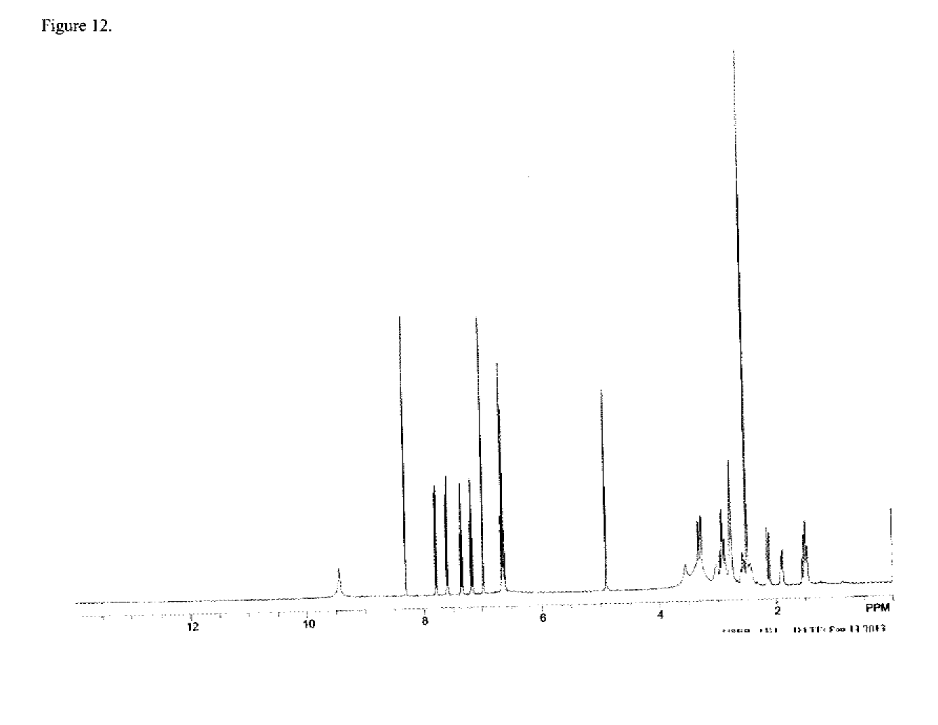

A solution of oxymorphone hydrochloride (2.93 g, 8.67 mmol) in water (31 g) was prepared by charging to a 100 mL beaker and stirring with a magnetic stir bar. The oxymorphone hydrochloride solution was then added dropwise to the sodium xinafoate solution prepared above via addition funnel over 10 minutes. A sticky solid formed and the reaction mixture was heated to 50° C. for 30 minutes which formed an oily layer. The solution was subsequently cooled to ambient temperature and stirred under nitrogen for 3.5 hours yielding a gummy solid in the reaction mixture. The water was decanted and the gum was washed with two 100 mL portions of water with the water decanted off each time. The isolate gum was dried overnight under reduced pressure to provide 1.68 g (40% yield) of a crunchy light yellow solid (KF 5.47% H₂O) which was characterized by DSC (FIG. 9), FTIR (FIG. 10), PXRD (FIG. 11), and ¹H-NMR (FIG. 12). The ¹H NMR spectrum was consistent with the expected 1:1 xinafoate salt and the PXRD diffractogram indicated the salt was amorphous.

### Example 4

### Preparation of Codeine Base Monohydrate

To a 150 mL beaker equipped with a magnetic stir bar was charged codeine sulfate trihydrate (3.0 g, 4.0 mmol) and water (99 g). To the slurry was added ammonium hydroxide solution (28%, 1.26 g, 10.1 mmol) and the solution stirred for 5 minutes.

The solution was transferred to a separatory funnel and extracted with three 70 g portions of ethyl acetate. The rotary evaporation of the combined organic layers yielded a solid which was further dried under reduced pressure to provide 2.31 g (91%) of a white solid which was characterized by DSC, FTIR and PXRD. PXRD indicated the product was crystalline.

### Example 5

### Preparation of Amorphous Codeine Pamoate (2:1)

To a 250 mL 3-neck round-bottom flask equipped with a mechanical stirrer, thermowell, nitrogen inlet and addition funnel was charged disodium pamoate (1.01 g, 2.25 mmol) and water (28 g). The solution's pH was adjusted to about 9 with 1 drop of 1N sodium hydroxide (90 mg) and the solution stirred under nitrogen.

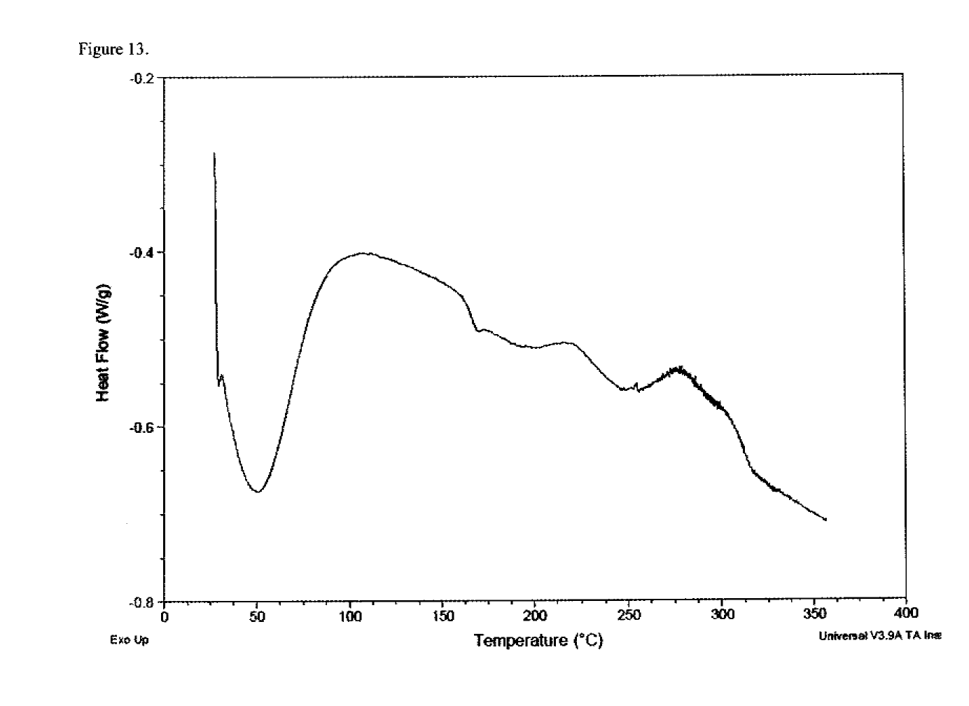

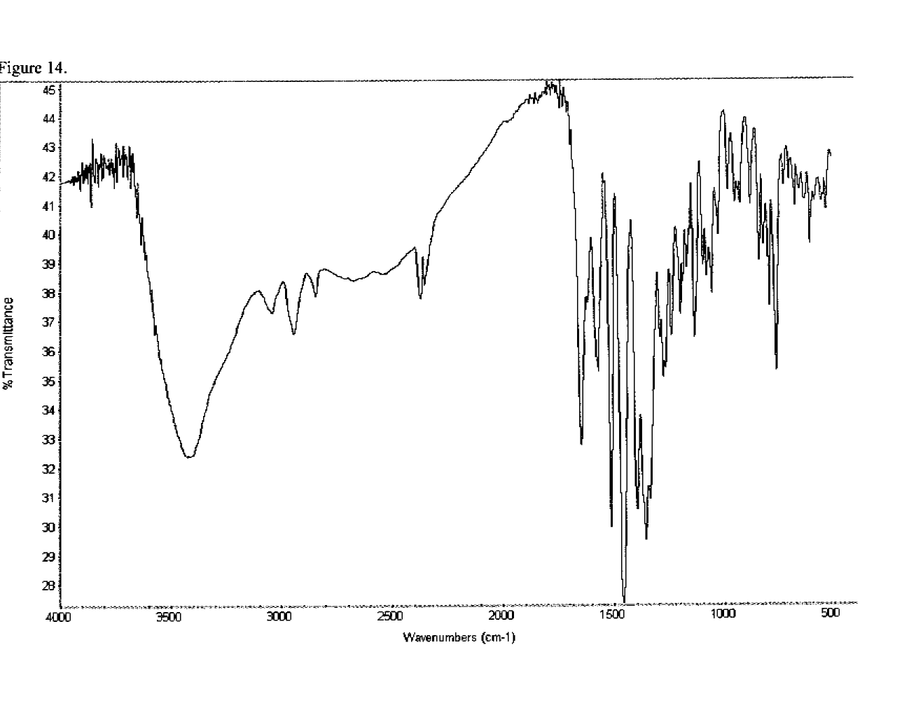

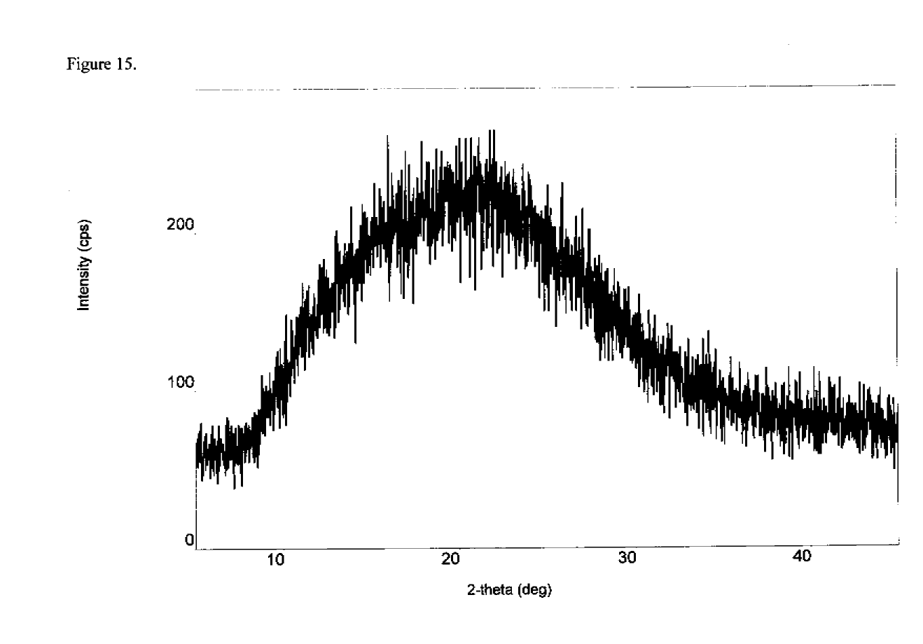

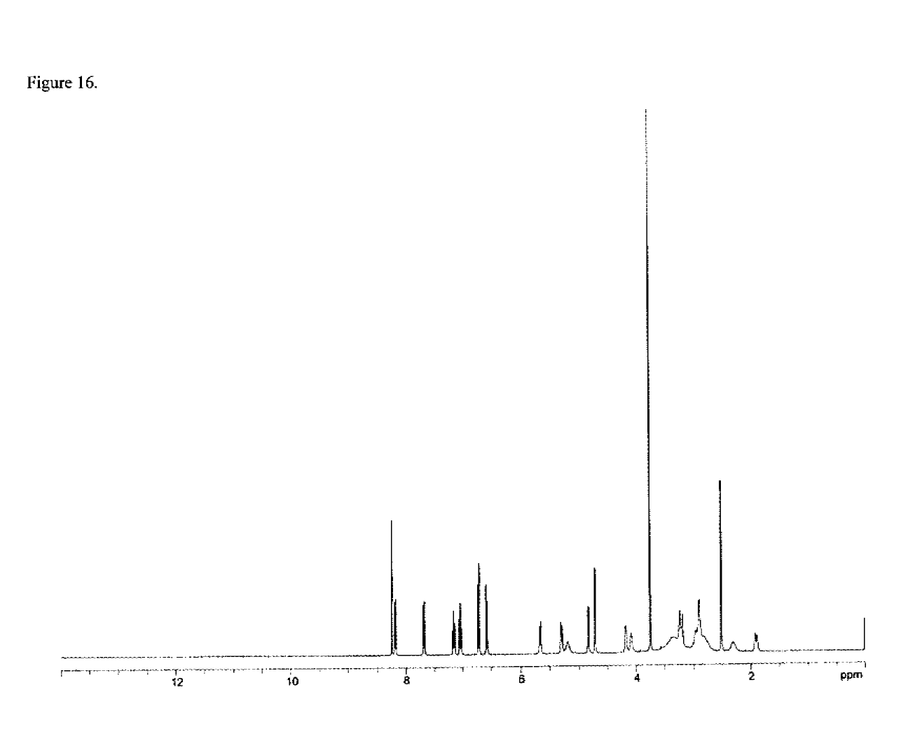

A solution of codeine hydrochloride was prepared by charging codeine base monohydrate (1.5 g, 4.73 mmol) as prepared in Example 4 and water (30 g) to a 100 mL beaker with stirring facilitated with a magnetic stir bar apparatus. A 1N hydrochloric acid solution (4.8 g, 4.73 mmol) was added to form codeine hydrochloride. The codeine hydrochloride solution was added dropwise to the disodium pamoate solution via addition funnel over 5 minutes. The resulting off-white slurry was stirred overnight at ambient temperature and the solids collected by filtration through a medium fritted filter and washed with four 20 mL portions of water. The product was dried overnight under reduced pressure to provide 2.13 g (96% yield) of an off-white solid (KF 6.18% H₂O) which was characterized by DSC (FIG. 13), FTIR (FIG. 14), PXRD (FIG. 15) and ¹H-NMR (FIG. 16). The ¹H-NMR spectrum was consistent with a compound having a 2:1 ratio of codeine to pamoate and the PXRD diffractogram indicated the salt was amorphous.

### Example 6

### Preparation of Codeine Xinafoate

To a 100 mL three-neck round bottom flask equipped with a mechanical stirrer, reflux condenser, thermowell and nitrogen inlet was charged BON acid (beta-oxy-naphthoic acid, also known as 2-hydroxyl-3-carboxy naphthalene) (942.8 mg, 5.01 mmol) and water (23 g). Sodium hydroxide (ACS grade, 200.4 mg, 5.01 mmol) was added and the mixture heated to 50° C. under nitrogen until all the solids dissolved. The sodium xinafoate solution was then cooled to ambient temperature.

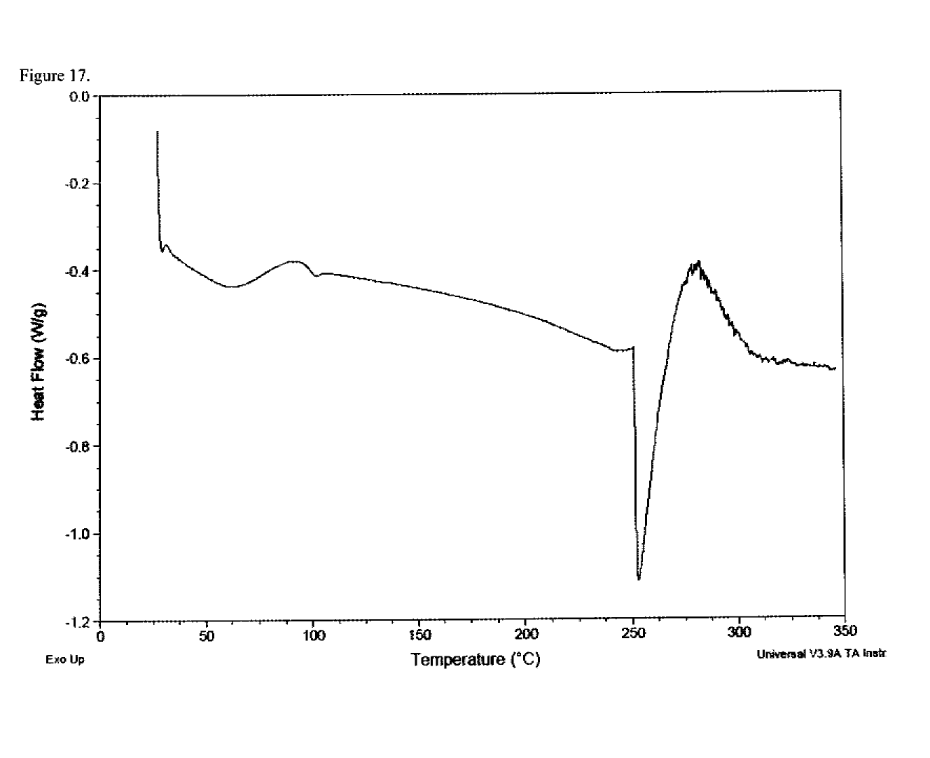

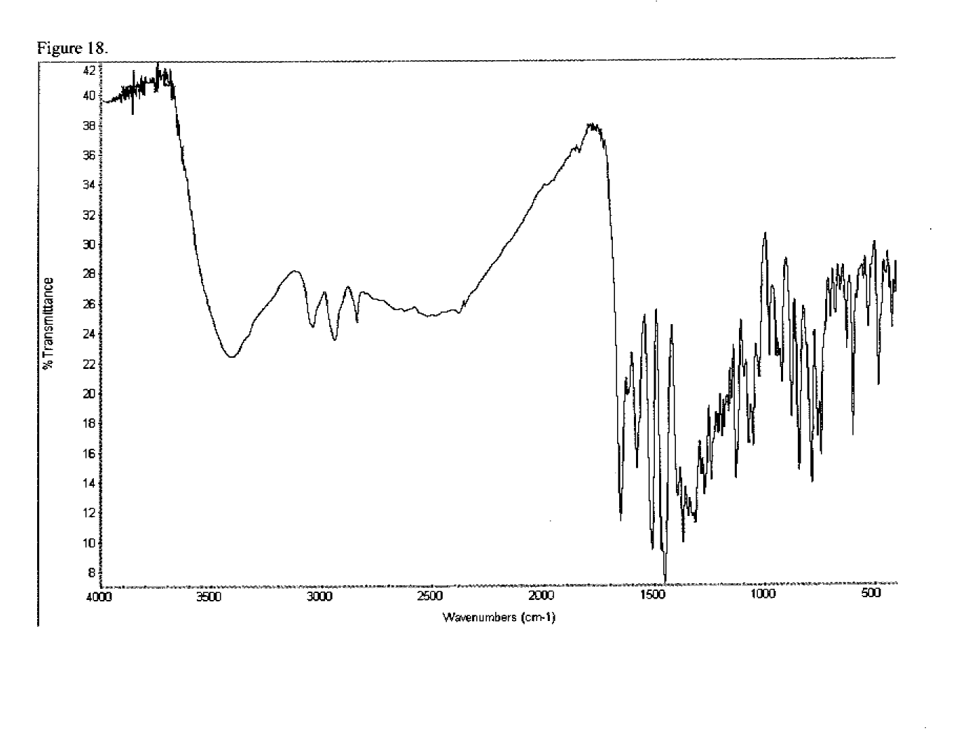

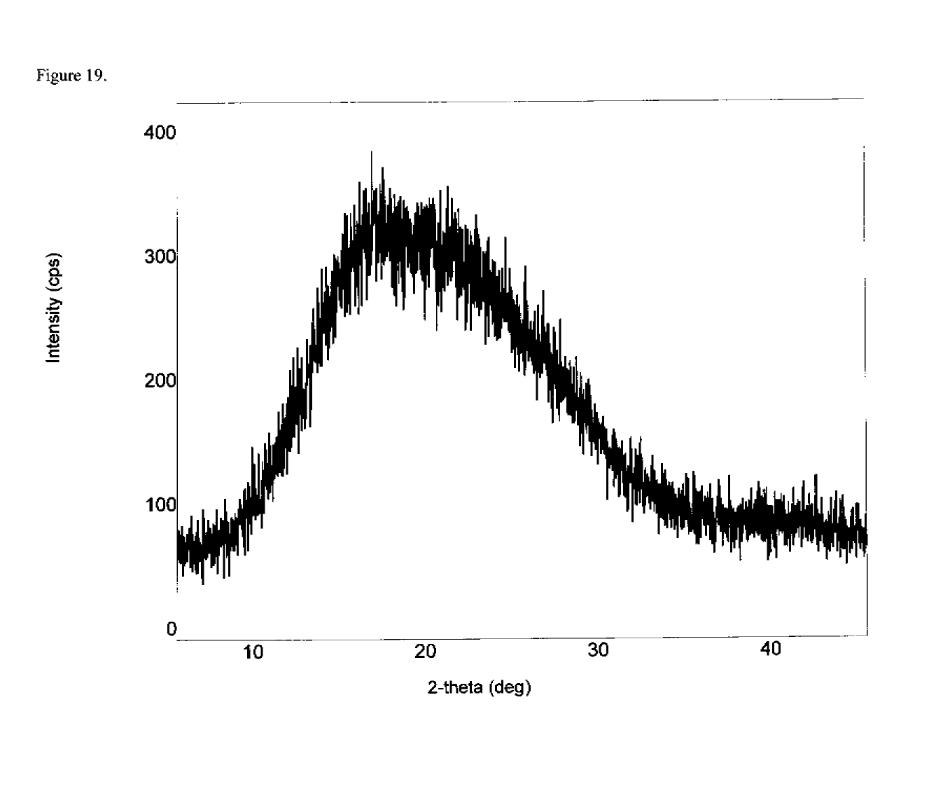

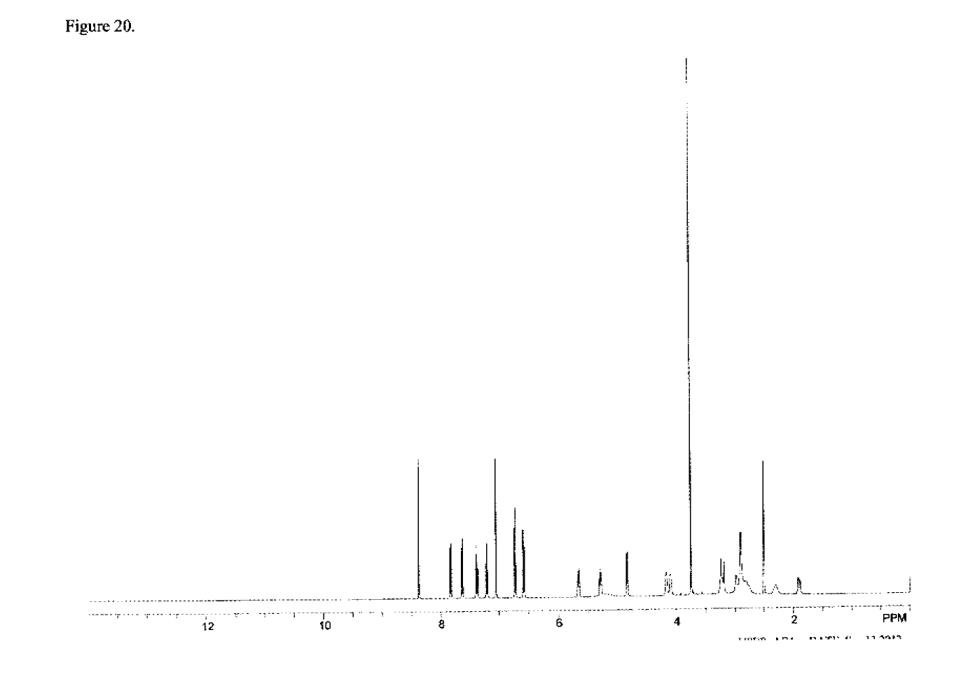

A solution of codeine hydrochloride in water was prepared in a 100 mL beaker by charging codeine base as prepared in Example 4 (1.5 g, 5.01 mmol) and water (15 g) and stirring with a magnetic stir bar apparatus with subsequent addition of 1N hydrochloric acid (5.10 g, 5.01 mmol). The codeine hydrochloride solution was added dropwise to the sodium xinafoate solution above via addition funnel over 5 minutes whereupon a sticky solid was formed. The reaction mixture was heated to 50° C. for 30 minutes which formed an oily layer. The solution was subsequently cooled to ambient temperature and stirred under nitrogen overnight resulting in the formation of a gummy solid. The water was decanted and the gum washed with two 100 mL portions of water. The gum was dried overnight under reduced pressure to provide 1.23 g (53% yield) of a crunchy light yellow solid (KF 3.63% H₂O) which was characterized by DSC (FIG. 17), FTIR (FIG. 18), PXRD (FIG. 19), and ¹H-NMR (FIG. 20). The ¹H-NMR spectrum was consistent with the expected 1:1 xinafoate salt and the PXRD diffractogram indicated the product was amorphous.

### Example 7

### Preparation of Amorphous Levorphanol Pamoate (2:1)

To a 100 mL 3-neck round-bottom flask equipped with a magnetic stir bar, thermowell, nitrogen inlet and addition funnel was charged disodium pamoate (242.1 mg, 0.538 mmol) and water (5.5 g). The solution was stirred under nitrogen.

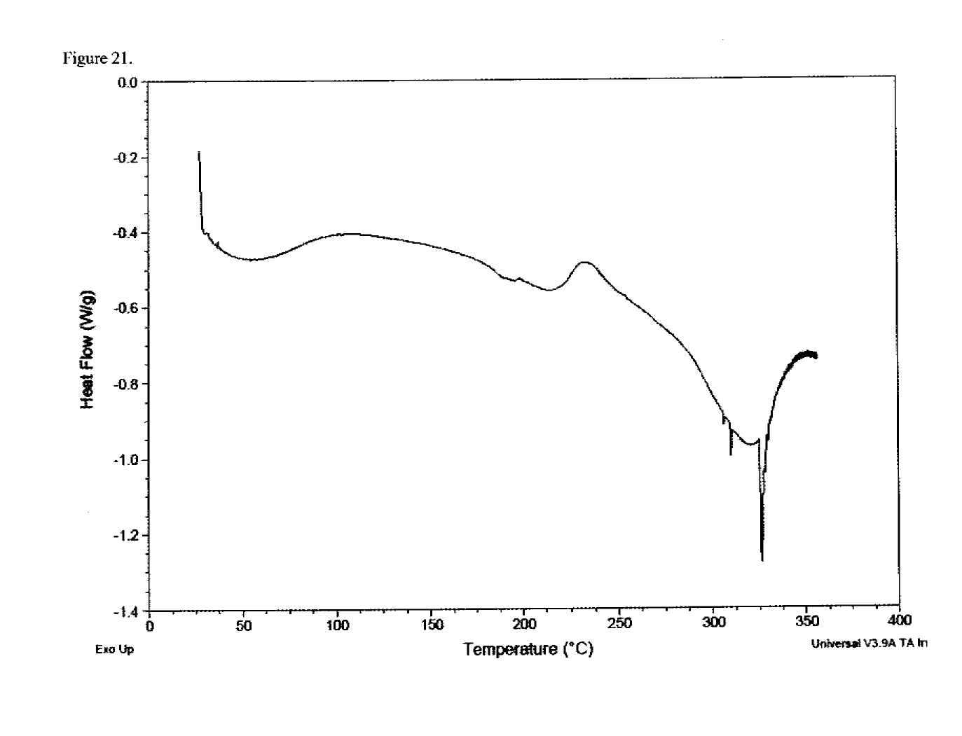

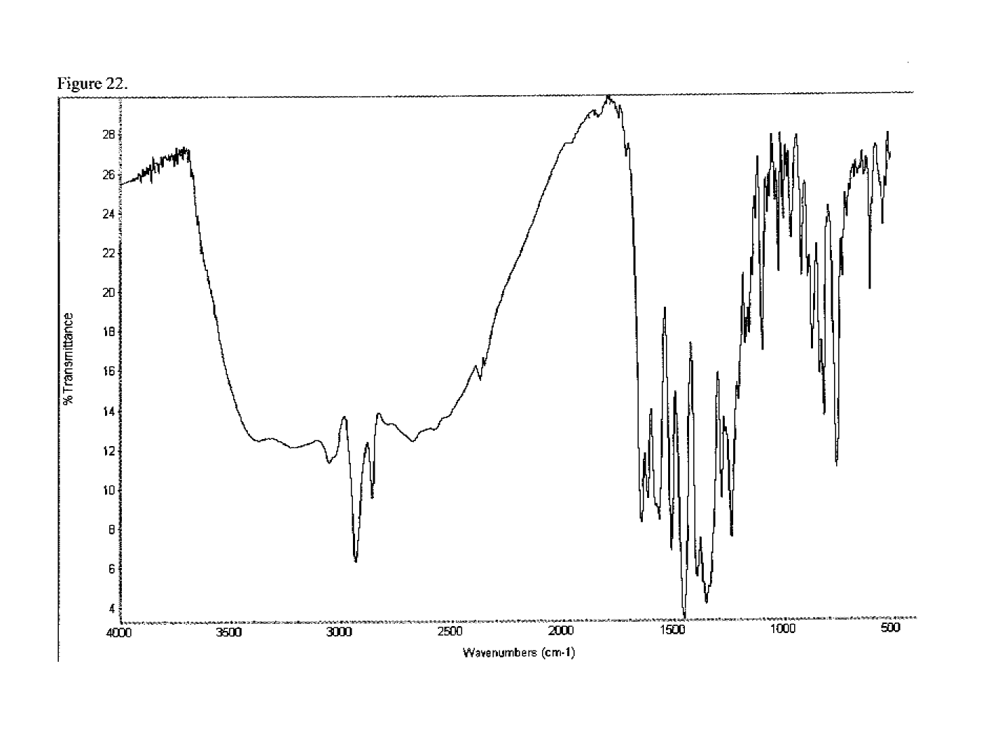

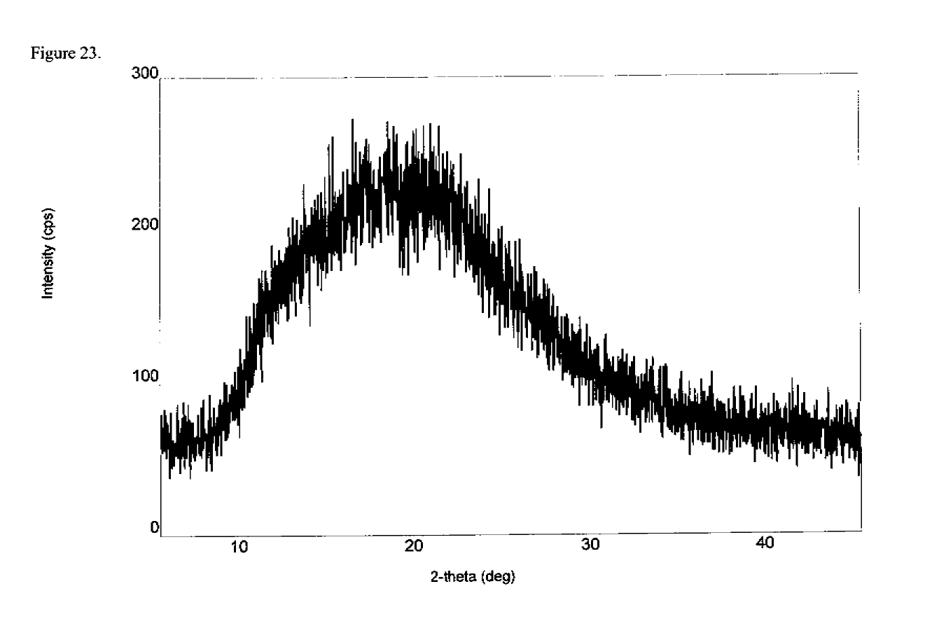

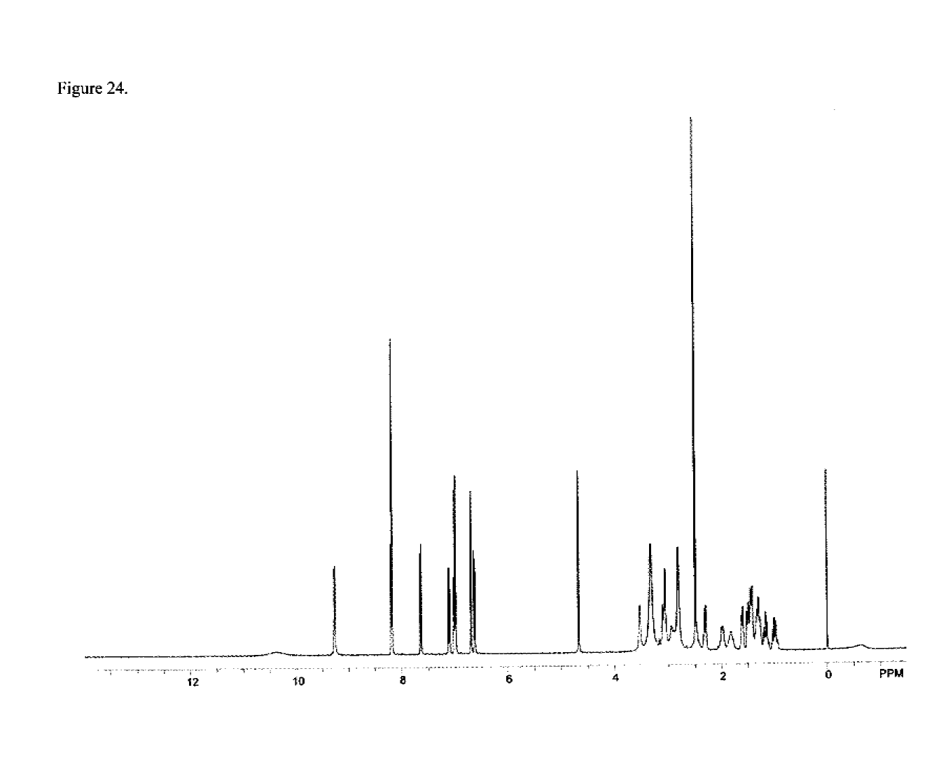

A solution of levorphanol tartrate dihydrate (0.5 g, 1.13 mmol) in water (22.9 g) was prepared by charging to a 50 mL beaker and stirring with a magnetic stir bar. The pH was 2.84. A 1N sodium hydroxide solution (1.21 g) was subsequently added to bring the pH to about 7.5. The resulting levorphanol solution was added dropwise to the disodium pamoate solution via addition funnel over 5 minutes. The resulting white slurry was stirred overnight at ambient temperature and the solids collected by filtration through a medium fritted filter and washed with four 5 mL portions of water. The product was dried overnight under reduced pressure to provide 0.43 g (88% yield) of an off-white solid (KF 3.39% H₂O) which was characterized by DSC (FIG. 21), FTIR (FIG. 22), PXRD (FIG. 23) and ¹H-NMR (FIG. 24). The ¹H-NMR spectrum was consistent with a structure possessing a 2:1 ratio of levorphanol to pamoate and the PXRD diffractogram indicated the salt was amorphous.

### Example 8

### Preparation of Polymorphic Levorphanol Pamoate (2:1)

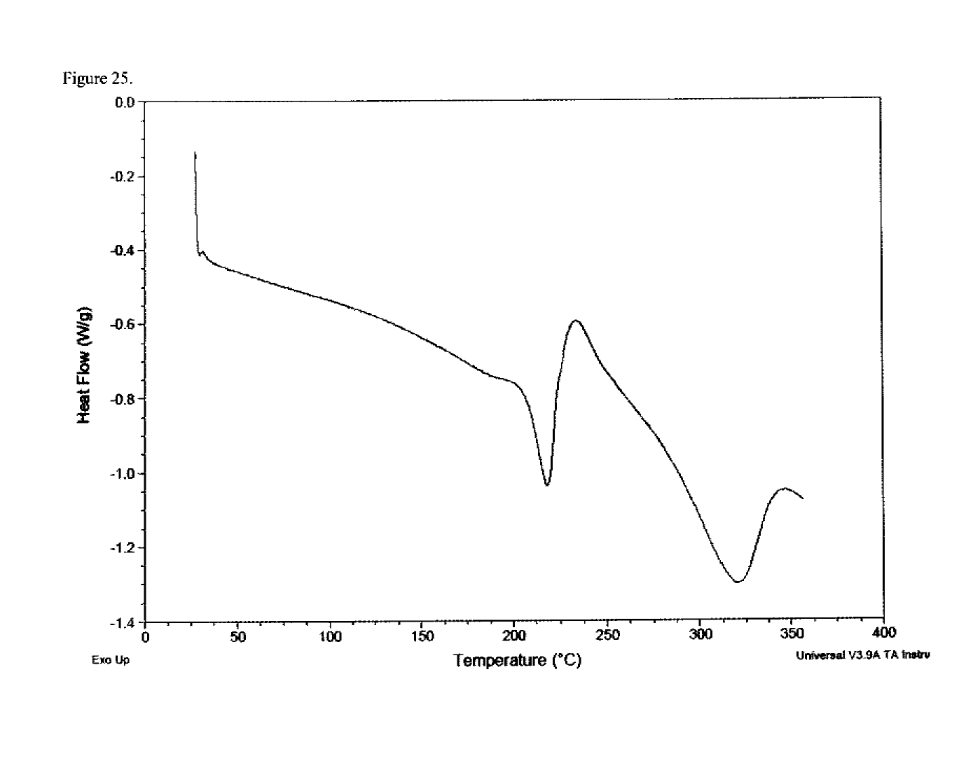

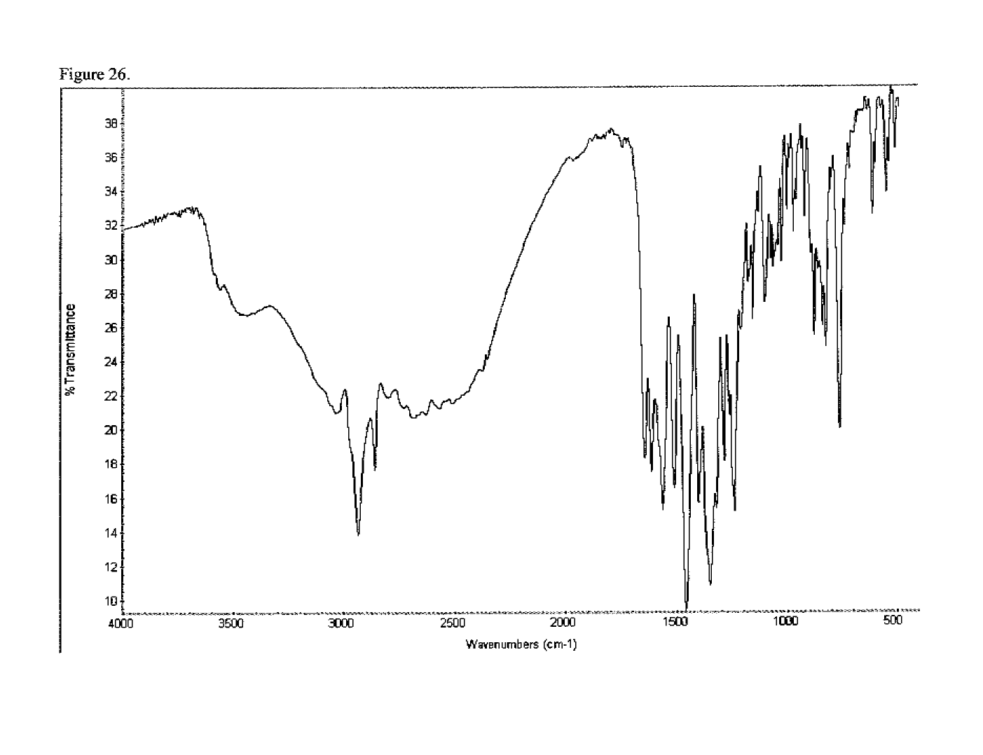

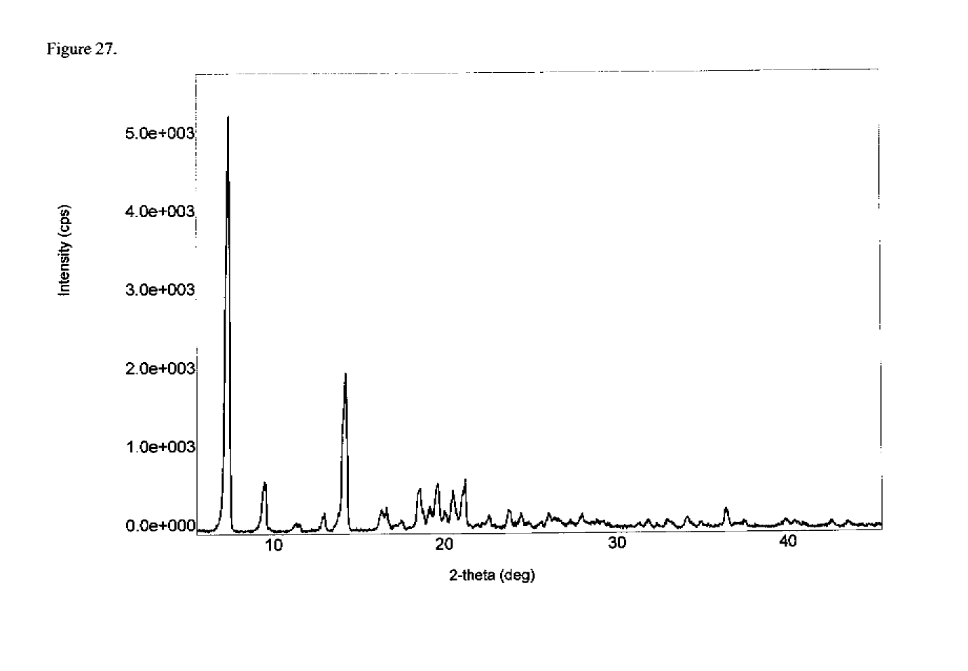

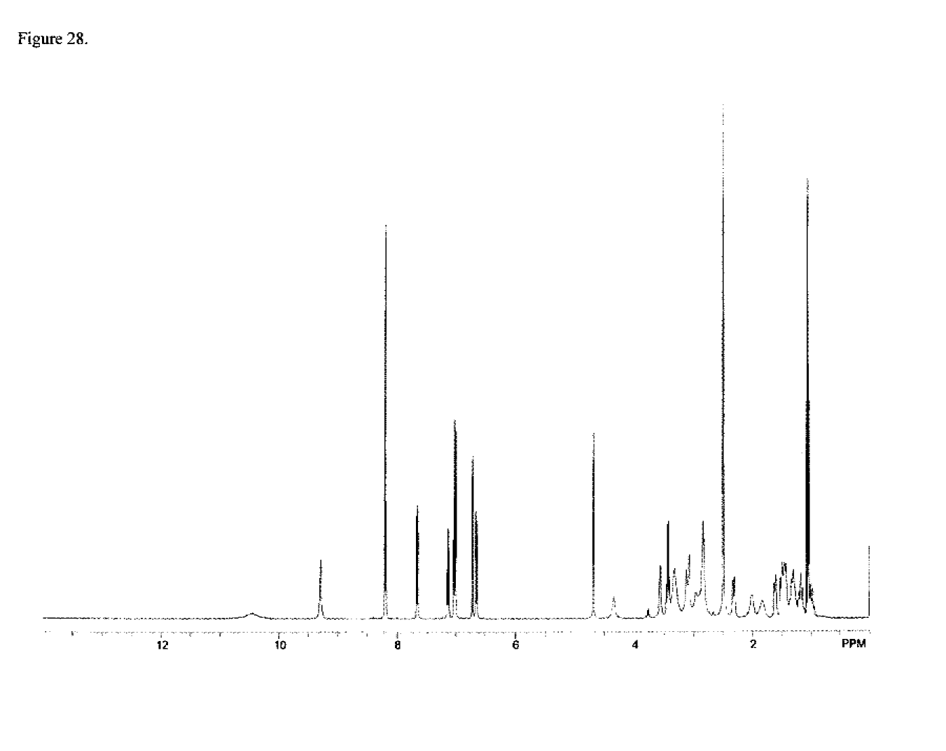

To a 50 mL three-neck round-bottom flask equipped with a magnetic stir bar, thermowell and nitrogen inlet was charged amorphous levorphanol pamoate (0.22 g, 0.24 mmol) and a 98:2 ethanol-water solution (10 g). The combined solution was heated to 76° C. under nitrogen overnight followed by solvent removal by rotary evaporation. An additional portion of 98:2 ethanol-water solution (10 g) was added and the mixture stirred at ambient temperature for several hours. The solids were collected by filtration through a medium fritted filter and washed with a very small portion of ethanol. The product was dried overnight under reduced pressure to provide 0.17 g (77% yield) of an off-white solid (KF 0.63% H₂O) which was characterized by DSC (FIG. 25), FTIR (FIG. 26), PXRD (FIG. 27) and ¹H-NMR (FIG. 28). The ¹H-NMR spectrum was consistent with a compound having a 2:1 ratio of levorphanol to pamoate and the PXRD diffractogram indicated the salt was crystalline.

### Example 9

### Preparation of Levorphanol Xinafoate

To a 100 mL three-neck round bottom flask equipped with a mechanical stirrer, reflux condenser, thermowell and nitrogen inlet was charged BON acid (beta-oxy-naphthoic acid, also known as 2-hydroxyl-3-carboxy naphthalene), (212.6 mg, 1.13 mmol) and water (23 g). Sodium hydroxide (ACS grade, 45.2 mg, 1.13 mmol) was added and the mixture heated to 50° C. under nitrogen until all the solids dissolved. The sodium xinafoate solution was then cooled to ambient temperature.

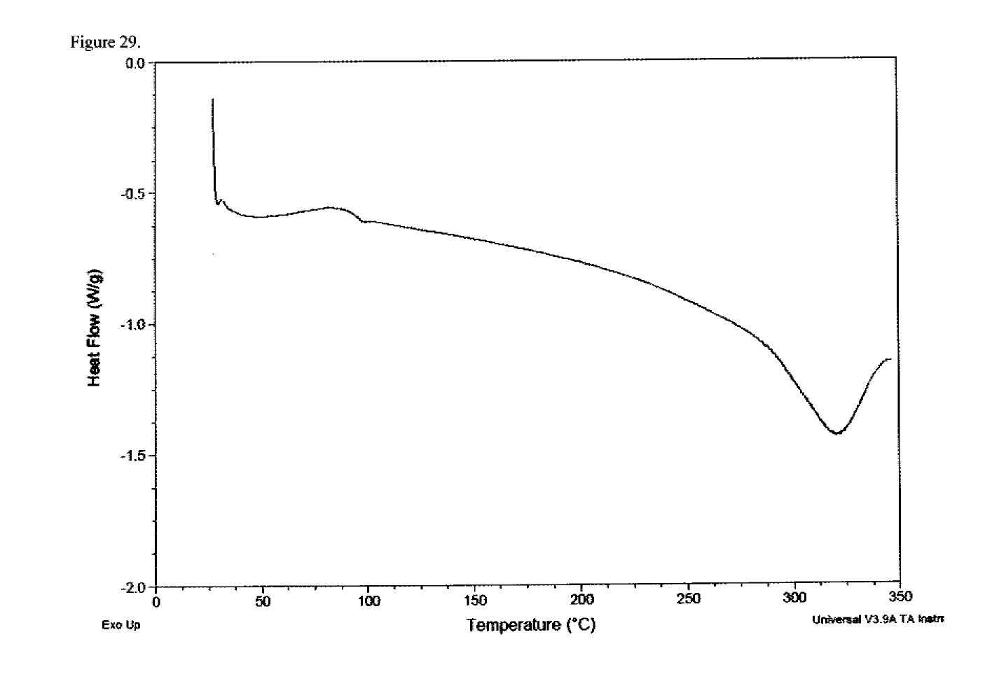

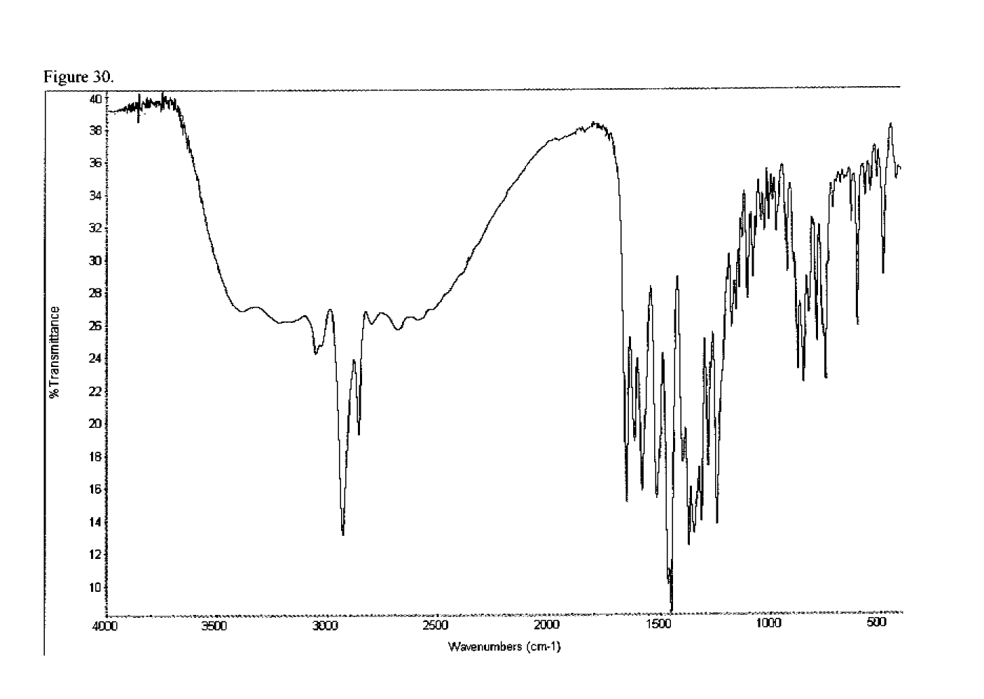

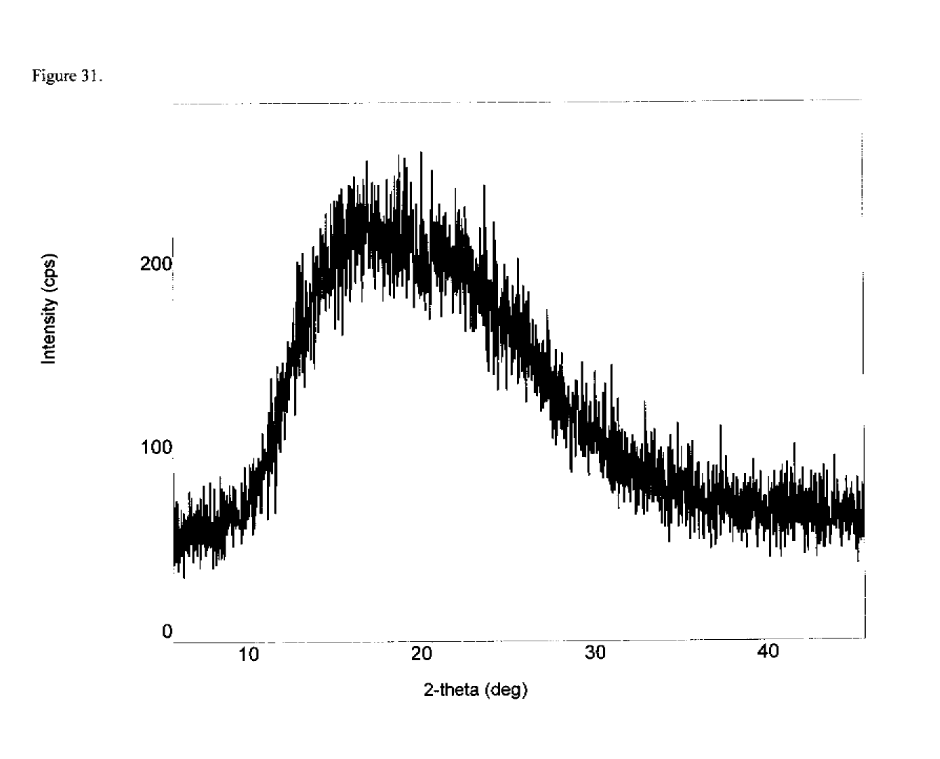

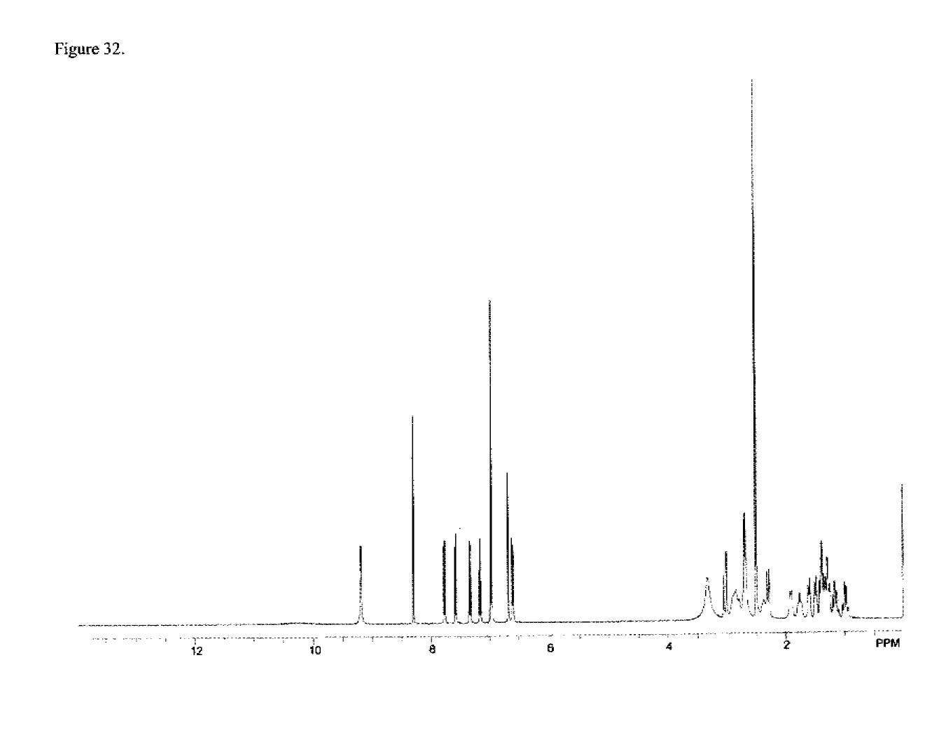

A solution of levorphanol tartrate dihydrate (0.5 g, 1.13 mmol) in water (23 g) was prepared by charging to a 50 mL beaker and stirring with a magnetic stir bar apparatus. The solution pH was about 3.6 and 1N sodium hydroxide solution (1.16 g) was subsequently added to bring the pH to about 7.9. The resulting levorphanol solution was added dropwise to the sodium xinafoate solution via addition funnel over 3 minutes. The resulting white slurry was stirred for 3.5 hours at ambient temperature and the solids collected by filtration through a medium fritted filter and then washed with four 5 mL portions of water. The product was dried overnight under reduced pressure to provide 0.3 g (60% yield) of an off-white solid (KF 3.10% H₂O) which was characterized by DSC (FIG. 29), FTIR (FIG. 30), PXRD (FIG. 31) and ¹H-NMR (FIG. 32). The ¹H-NMR spectrum was consistent with the expected 1:1 salt and the PXRD diffractogram indicated the salt was amorphous.

### Example 10

### Preparation of Amorphous Naltrexone Pamoate (2:1)

To a 100 mL 3-neck round-bottom flask equipped with a mechanical stirrer, thermowell, nitrogen inlet and addition funnel was charged disodium pamoate (567.0 mg, 1.26 mmol) and water (11 g). The reaction mixture pH was adjusted to about 9 with 1 drop of 1 N sodium hydroxide (40 mg) and stirred under nitrogen.

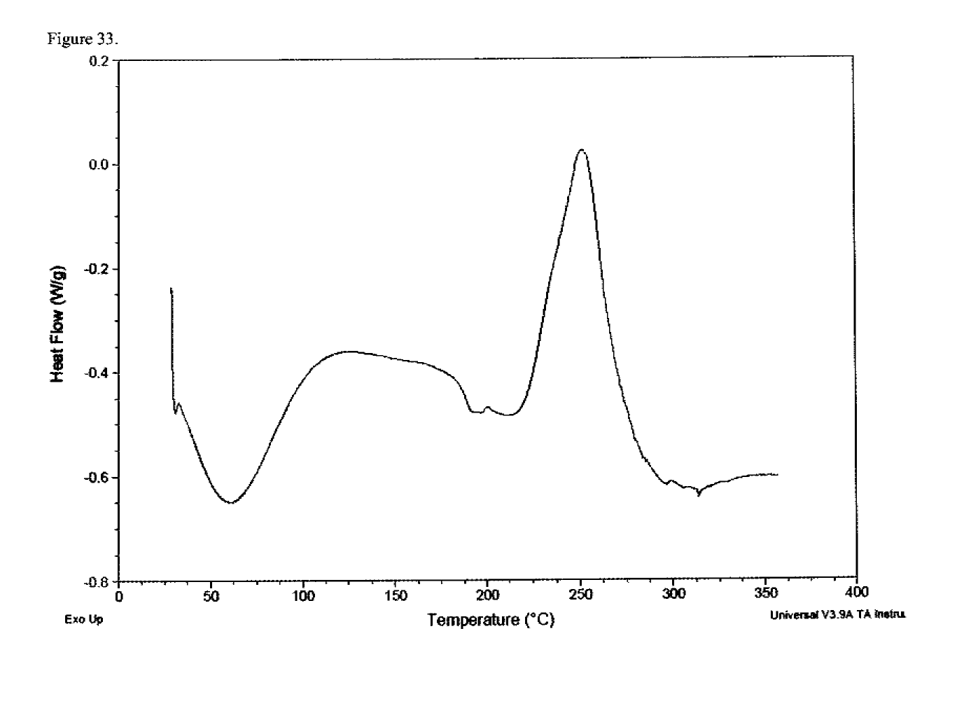

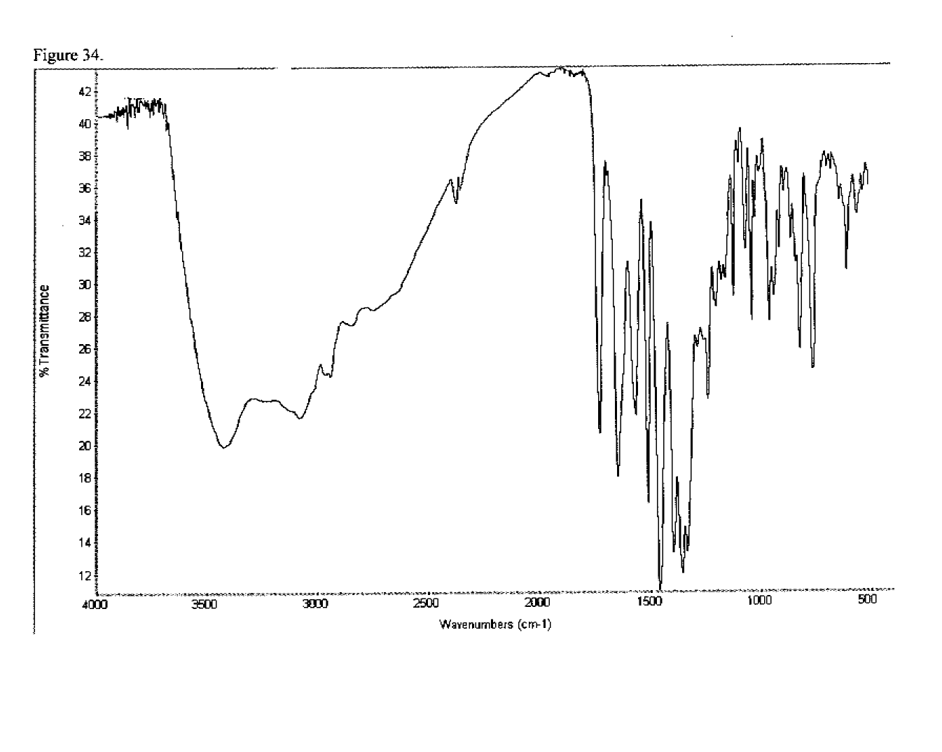

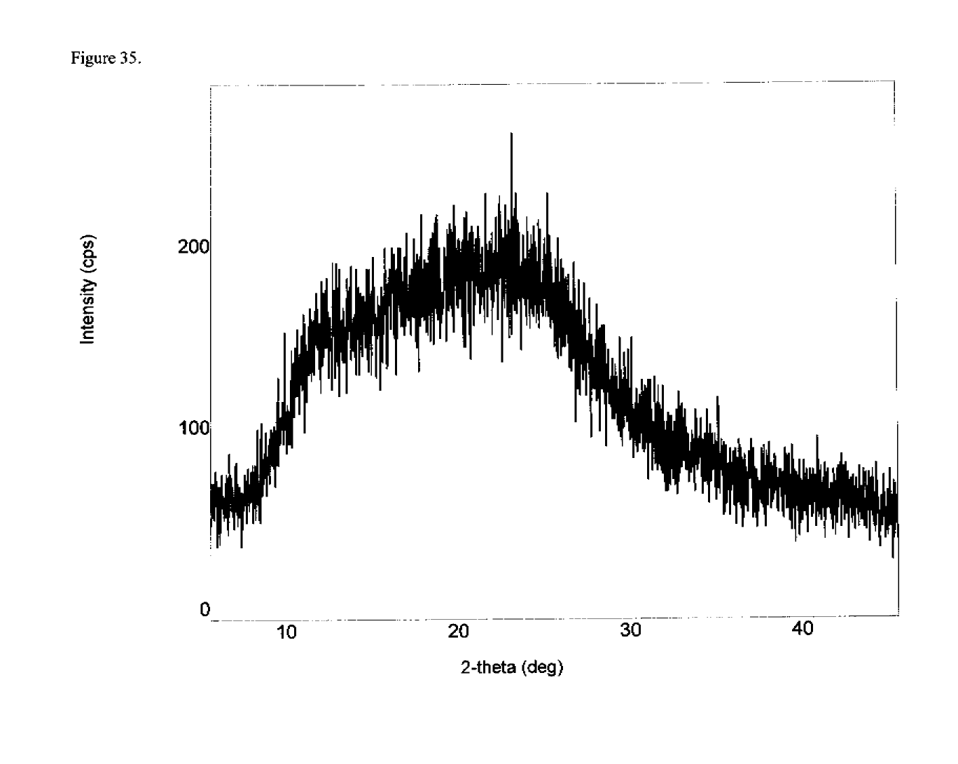

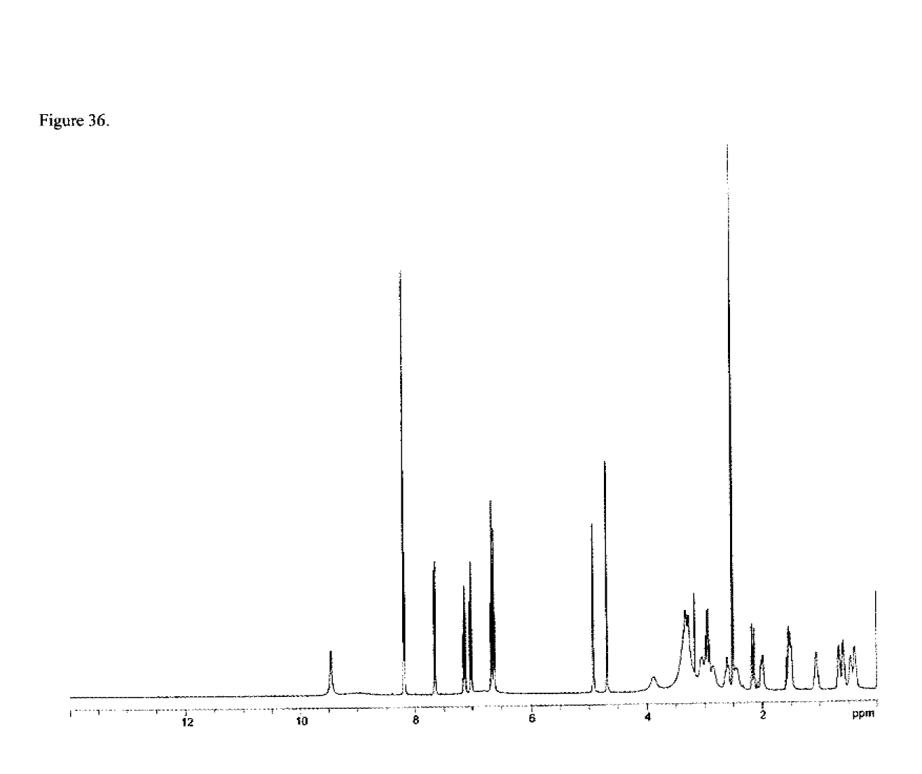

A solution of naltrexone hydrochloride (1.0 g, 2.65 mmol) in water (20 g) was prepared by charging to a 50 mL beaker and stirring with a magnetic stir bar apparatus. The naltrexone hydrochloride solution was added dropwise to the disodium pamoate solution via addition funnel over 5 minutes. The resulting off-white slurry was difficult to stir and an additional 10 g water added to facilitate stirring. The resulting mixture was stirred overnight at ambient temperature and the solids collected by filtration through a medium fritted filter and washed with four 20 mL portions of water. The product was dried overnight under reduced pressure to provide 1.16 g (86% yield) of an off-white solid (KF 6.08% H₂O) which was characterized by DSC (FIG. 33), FTIR (FIG. 34), PXRD (FIG. 35) and ¹H-NMR (FIG. 36). The ¹H-NMR spectrum was consistent with a compound possessing a 2:1 ratio of naltrexone to pamoate and the PXRD diffractogram indicated the salt was amorphous.

### Example 11

### Preparation of Polymorphic Naltrexone Pamoate 2:1

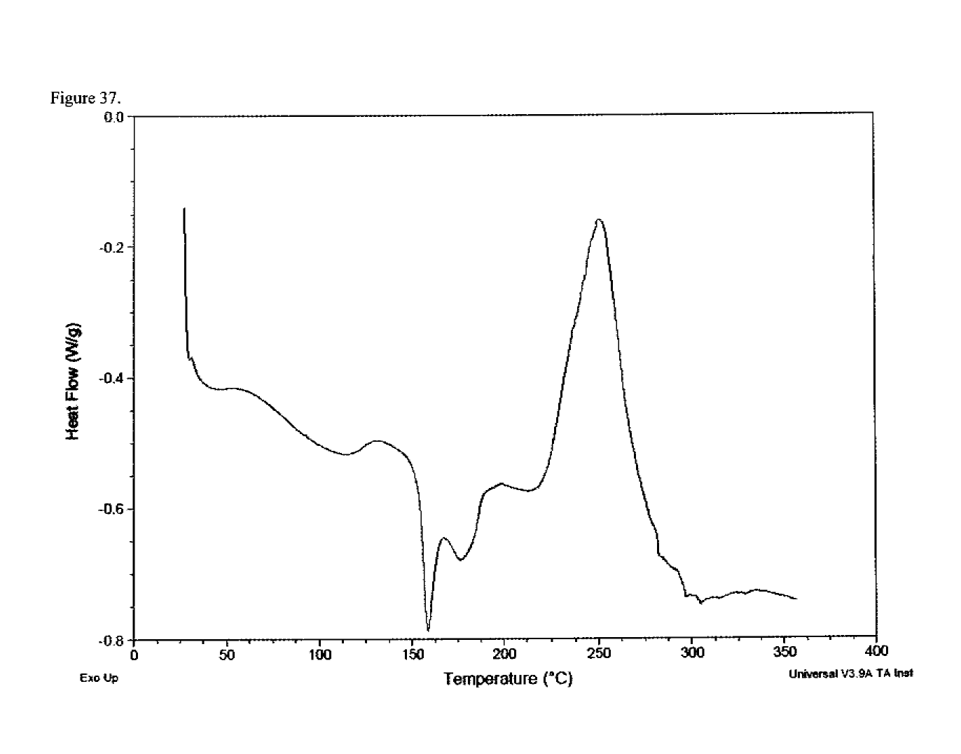

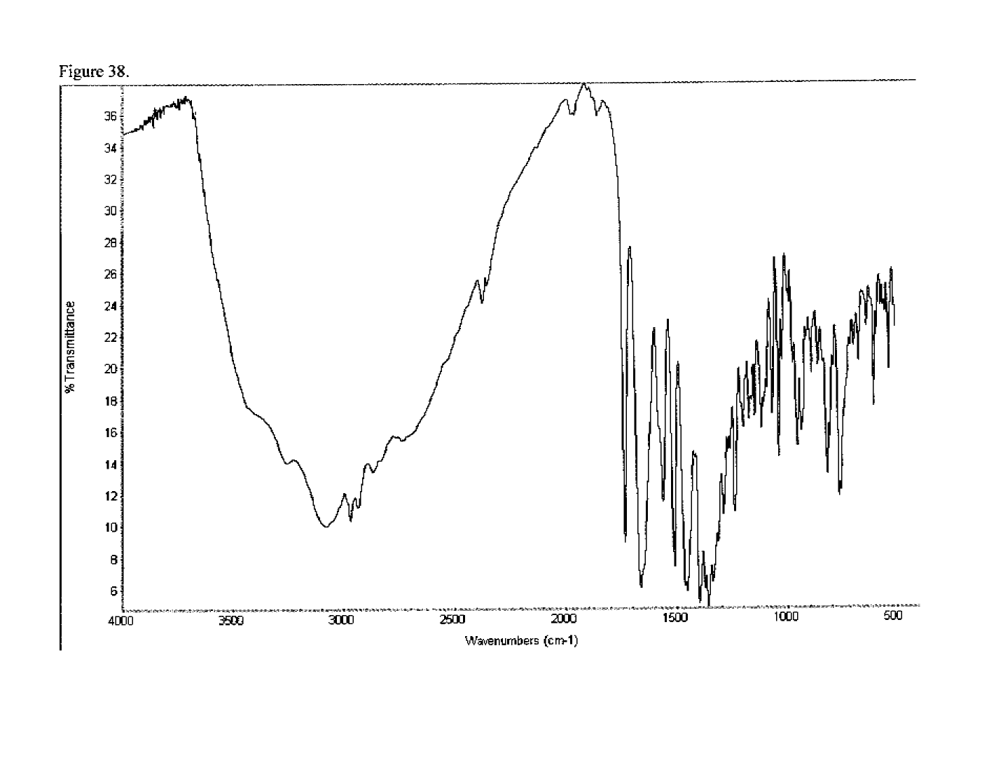

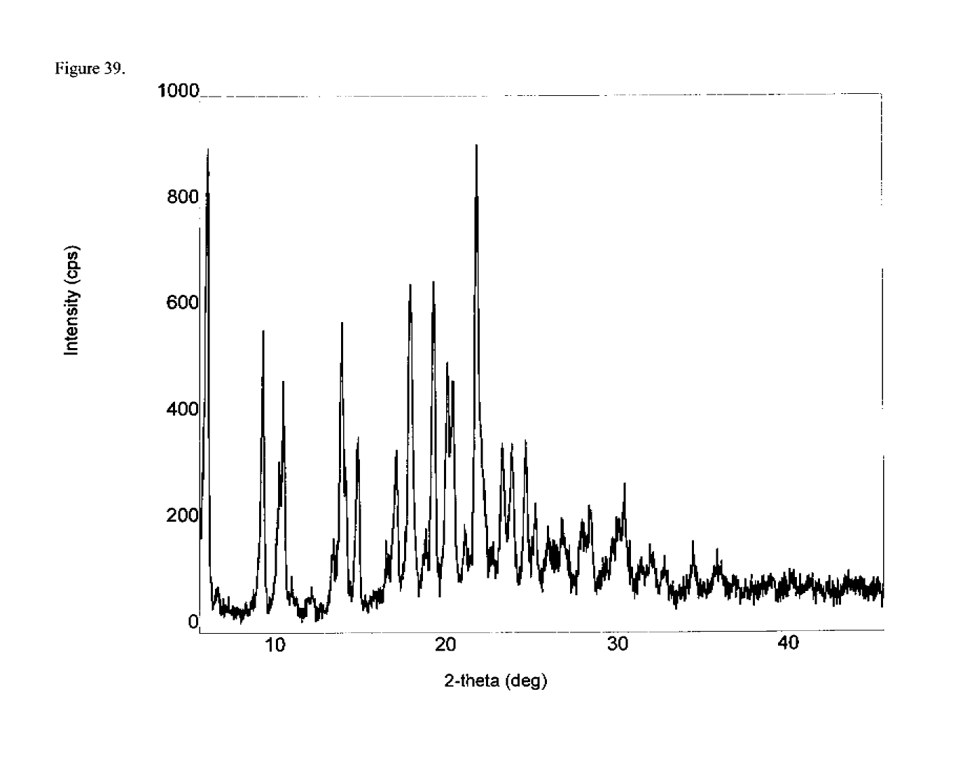

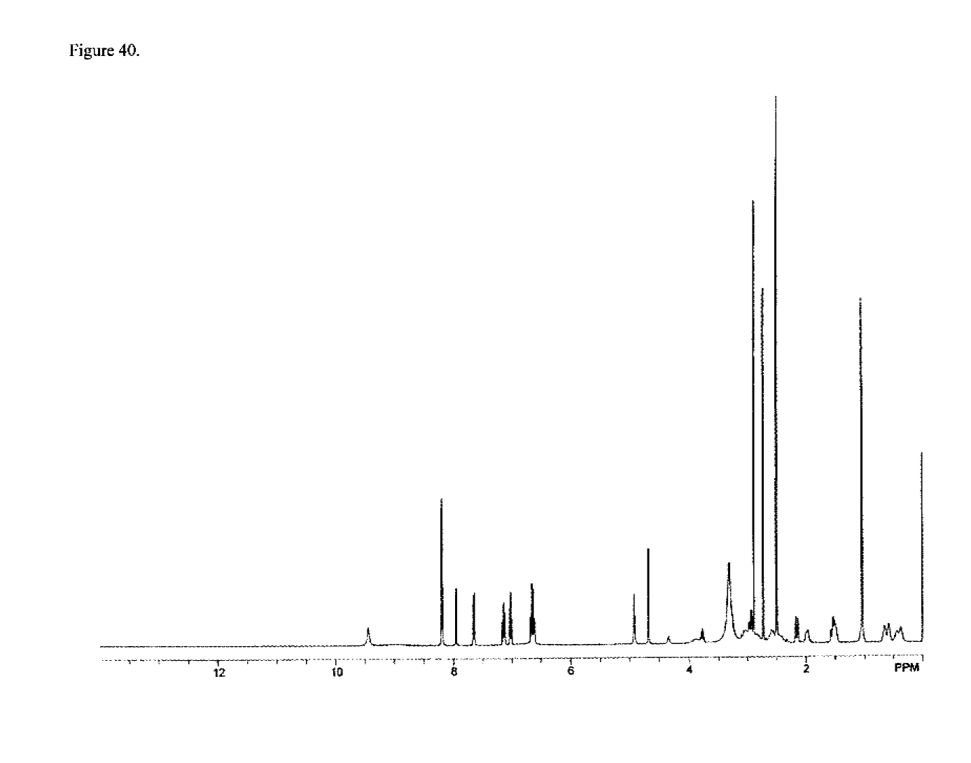

To a 50 mL 3-neck round-bottom flask equipped with a magnetic stir bar, thermowell and nitrogen inlet was charged amorphous naltrexone pamoate (0.41 g, 0.383 mmol) and DMF (1.6 g) to form a solution. Isopropanol (4.9 g) was added dropwise and the resulting slurry was stirred overnight at ambient temperature. The solids were collected by filtration through a medium fritted filter and washed with a very small portion of isopropanol. The product was dried overnight under reduced pressure to provide 0.32 g (78% yield) of an off-white solid (KF 3.06% H₂O) which was characterized by DSC (FIG. 37), FTIR (FIG. 38), PXRD (FIG. 39), and ¹H-NMR (FIG. 40). The ¹H-NMR spectrum was consistent for a compound having a 2:1 ratio of naltrexone to pamoate and the PXRD diffractogram indicated the salt was crystalline.

### Example 12

### Preparation of Naltrexone Xinafoate

To a 100 mL three-neck round bottom flask equipped with a mechanical stirrer, reflux condenser, thermowell and nitrogen inlet was charged BON acid (beta-oxy-naphthoic acid, also known as 2-hydroxyl-3-carboxy naphthalene), (1.63 g, 8.67 mmol) and water (23 g). Sodium hydroxide (ACS grade, 346.8 mg, 8.67 mmol) was added and the mixture heated to 50° C. under nitrogen until all the solids dissolved. The sodium xinafoate solution was then cooled to ambient temperature.

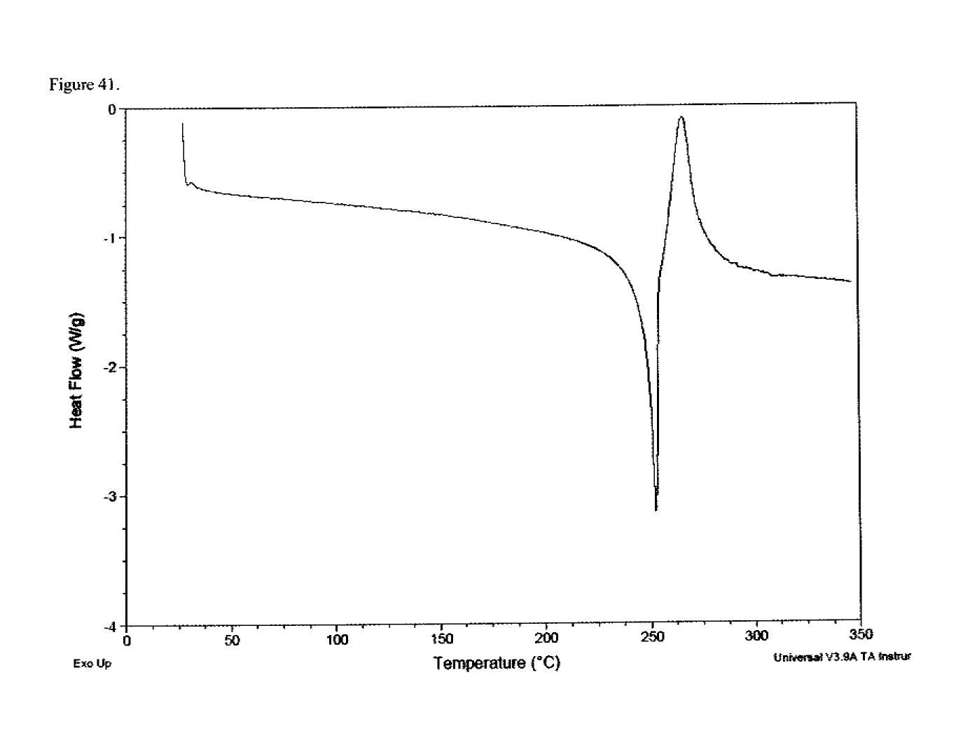

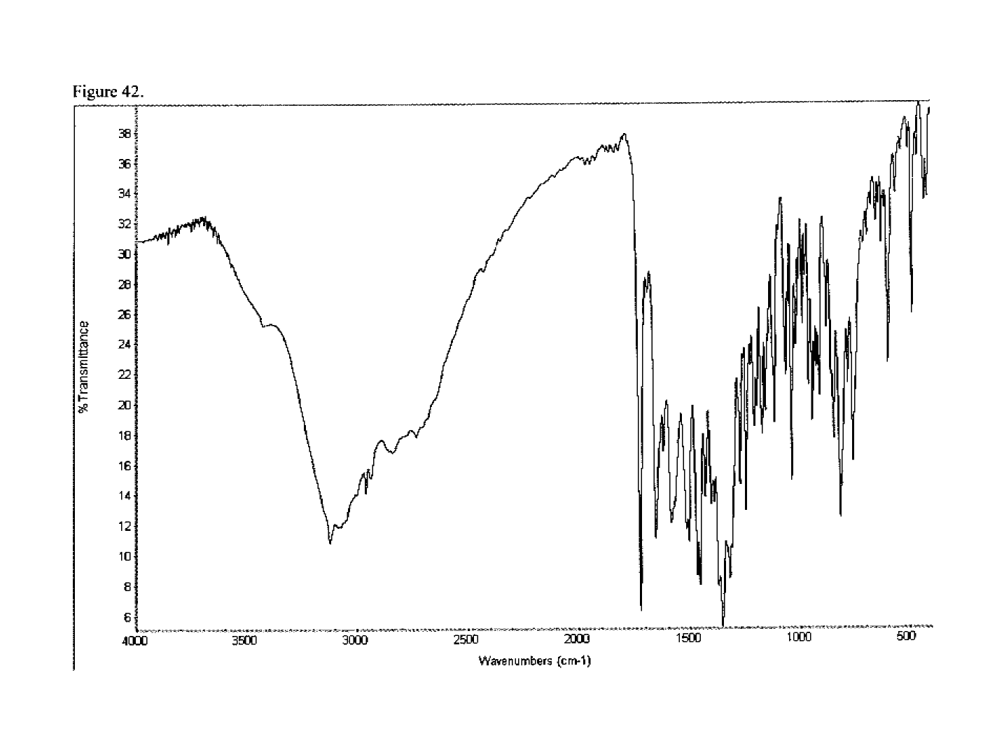

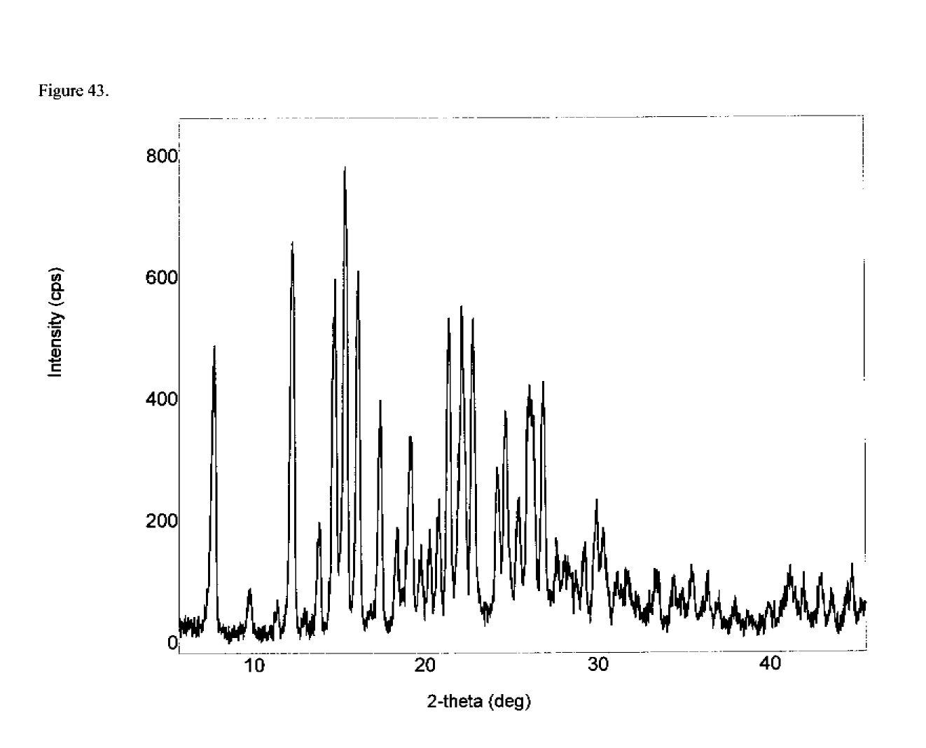

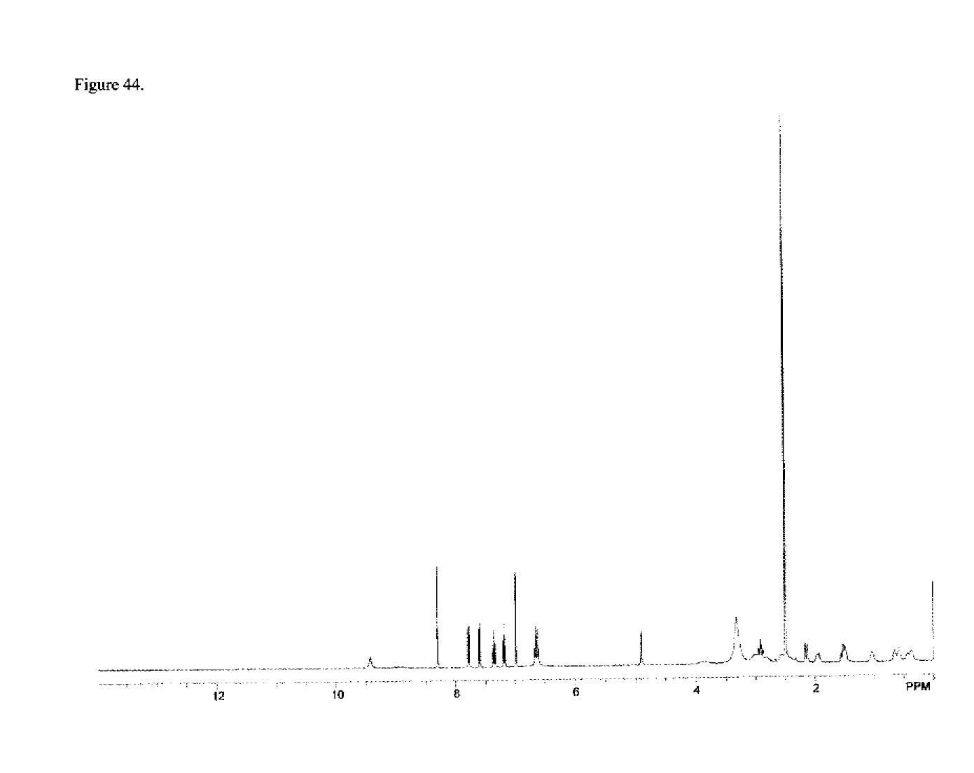

A solution of naltrexone hydrochloride (3.28 g, 8.67 mmol) in water (62 g) was prepared by charging to a 100 mL beaker and stirring with a magnetic stir bar apparatus. The naltrexone hydrochloride solution was then added dropwise to the sodium xinafoate solution prepared above via addition funnel over 10 minutes. The resulting slurry was stirred overnight under nitrogen. The solids were collected by filtration through a medium fritted filter, washed with two 100 mL portions of water and the product dried under reduced pressure to provide 2.86 g (62% yield) of a white solid (KF 0.15% H₂O) which was characterized by DSC (FIG. 41), FTIR (FIG. 42), PXRD (FIG. 43) and ¹H-NMR (FIG. 44). The ¹H-NMR spectrum was consistent with the expected 1:1 salt and the PXRD diffractogram indicated the salt was crystalline

### Example 13

### pH and Dose Dumping Dissolution Procedures

The amine containing organic acid addition salts of the present invention were tested to determine their dissolution profile as a function of pH, and as a function of ethanol concentration in acidic media (dose dumping). To perform these experiments the buffered dissolution media and acidic ethanol solutions were prepared as identified herein, “Preparation of Solutions”. The test procedure was derived from the procedures cited in the United States Pharmacopeia and National Formulary (USP), numbers <1087> and <711>. The dose dumping procedure was adopted from the United States Food and Drug Administration's guidance regarding the dose dumping of oxymorphone. The sampling interval and regimen was defined and each sample analyzed by HPLC. Results from the HPLC analyses were plotted as a function of time and dissolution condition (FIG. 24 through FIG. 37 inclusive). This procedure was used to obtain the pH and dose dumping dissolution profiles disclosed herein. Verb tense within the tense within the procedure description does not indicate a prospective condition but was used to facilitate the method's description herein. All activities within the procedure were conducted and executed for each of the compounds reported herein.

### Dissolution Procedure

The analytical methodology described in detail in the Experimental section for determining the pH and dose dumping dissolution profiles relies on HPLC methodology to quantify the analytes. Typically, the principal analyte being monitored is the specific active ingredient, i.e. oxymorphone; however, the separations methodology of HPLC also allows for quantification of the pamoate moiety too. Interestingly, the pamoate moiety provides an analysis and interpretation complication. Independently graphing the analytes, oxymorphone and pamoate, to provide species-specific dissolution profiles may, at first, offer a conflicting result. Under acidic conditions, the oxymorphone species may show significant release as a function of time whereas the corresponding pamoate dissolution profile indicates limited release. This is easily explained upon recognition that the pamoate moiety precipitates as pamoic acid and consequently its quantification within dissolution samples subjected to HPLC analysis is quite low despite correspondingly higher levels of the active ingredient. Conversely, the pamoate moiety in its ionic form, for instance at buffer pH 6.8 and greater, is reasonably soluble. Discernment is required to realize that oxymorphone pamoate may have an inhibited dissolution profile in this pH range and indeed, monitoring the pamoate dissolution indicates only low levels of release.

The following is a general procedure for intrinsic dissolution experiments.

### Preparation of Solutions:

All reagents are ACS grade or equivalent. All solvents used are a minimal of HPLC grade. Water used in the preparations of all solutions is USP grade. These solution preparations have been taken directly from the USP.

### Preparation of 0.1 N HCl:

To prepare 4 L of solution, add 33.3 mL of concentrated HCl to 977.7 mL of water, then add an additional 3000 mL of water.

### Preparation of pH 4.5 Acetate Buffer:

To prepare 1 L of solution add 2.99 g of sodium acetate tri-hydrate (NaC₂H₃O₂. 3H₂O) to a 1000 mL volumetric flask, then add 14.0 mLs of 2N acetic acid solution. Dissolve and dilute to volume with water.

### Preparation of pH 6.8 Phosphate Buffer:

To prepare 200 mL of solution first prepare a 0.2 M potassium phosphate solution by adding 27.22 g of monobasic potassium phosphate (KH₂PO₄) to a 1000 mL volumetric flask, then dissolve and dilute to volume with water. Add 50 mL of this solution to a 200 mL volumetric flask, then add 22.4 mL of 0.2M NaOH and dilute to volume with water.

### Preparation of 5% Ethanol Solution for Dose Dumping Dissolution Profiles:

To prepare 900 mL of media combine 45 mL of 200 proof ethanol with 855 mL of 0.1 N HCl (see preparation procedure above).

### Preparation of 20% Ethanol Solution for Dose Dumping Dissolution Profiles:

To prepare 900 mL of media combine 180 mL of 200 proof ethanol with 720 mL of 0.1 N HCl (see preparation procedure above).

### Preparation of 40% Ethanol Solution for Dose Dumping Dissolution Profiles:

To prepare 900 mL of media combine 360 mL of 200 proof ethanol with 540 mL of 0.1 N HCl (see preparation procedure above).

### Preparation of Mobile Phase A (0.1% TFA in H₂O):

To prepare 1 L of mobile phase, add 1.0 mL of TFA (trifluoroacetic acid) to 1000 mL of H₂O. Mix well and filter this solution through a 0.45 μM nylon filter.

### Preparation of Mobile Phase B (0.1% TFA in Acetonitrile):

To prepare 1 L of mobile phase, add 1.0 mL of TFA to 1000 mL acetonitrile. Mix well and filter this solution through a 0.45 μM nylon filter.

### Preparation of Mobile Needle/Seal Wash Solution:

To prepare 1 L of solution, add 500 mL H₂O to 500 mL acetonitrile and mix well.

### Procedures:

### Intrinsic Dissolution Profiles:

Note: The following procedures were derived from USP <1087> Intrinsic Dissolution and USP <711> Dissolution methods, as well as manufacturer recommended procedures for use of the Distek Inc. intrinsic dissolution disks.

### Preparation of API Pellet for Intrinsic Dissolution:

The material which is to be subjected to dissolution is weighed using an analytical balance. 45.00-65.00 mgs of the analyte was weighed and transferred to a Distek Inc. fixed/static disk 316 stainless die with a 0.8 cm diameter die cavity. A hardened steel punch was then inserted into the cavity and the material was compressed at 2000 psi for 4-5 minutes using a bench top hydraulic press. A Viton gasket is then placed around the threaded shoulder of the die and a polypropylene cap is threaded onto the die. This process can be repeated to generate as many pellets as is necessary for the experiment. The die is placed in the dissolution vessel such that the 0.5 cm² pellet surface is exposed to the dissolution media.

### Setup of Intrinsic Dissolution Apparatus:

A Hansen Research SR8 Plus Dissolution Test Station was filled with water and set to a temperature of 37.2° C. The vessel cavities were then equipped with four 1 L flat-bottomed Distek dissolution vessels. Four vessels were then filled with 600 mL of the following media: 0.1 N HCl, pH 4.5 acetate buffer, pH 6.8 phosphate buffer, and USP grade water. The solutions were allowed to warm in the water bath for approximately 1 hour, but not exceeding 3 hours, or until the temperature of the media matched that of the water bath. Paddles were then mounted to the Hansen Dissolution Test Station stirring apparatus above the four dissolution vessels such that the distance between the paddle and the die face is 1 inch. The paddle speed is then set to 50 RPM.

Note: The following procedures were derived from the FDA Draft Guidance for Oxymorphone Hydrochloride (recommended in November, 2007).

Preparation of API Pellet for Intrinsic Dissolution Dose Dumping Profile: The material which is to be subjected to dissolution is weighed using an analytical balance. 45.00-65.00 mgs of the analyte was weighed and transferred to an Distek Inc. fixed/static disk 316 stainless die with a 0.8 cm diameter die cavity. A hardened steel punch was then inserted into the cavity and the material was compressed at 2000 psi for 4-5 minutes using a bench top hydraulic press. A Viton gasket is then placed around the threaded shoulder of the die and a polypropylene cap is threaded onto the die. This process can be repeated to generate as many pellets as is necessary for the experiment. The die is placed in the dissolution vessel such that the 0.5 cm² pellet surface is exposed to the dissolution media.

### Setup of Intrinsic Dissolution Apparatus for Dose Dumping Profile:

A Hansen Research SR8 Plus Dissolution Test Station was filled with water and set to a temperature of 37.2° C. The vessel cavities were then equipped with four 1 L flat-bottomed Distek dissolution vessels. The vessels were then filled with 900 mL of the following media: 0.1 N HCl, 5% ethanol solution, 20% ethanol solution, and 40% ethanol solution. The solutions were allowed to warm in the water bath for approximately 1 hour, but not exceeding 3 hours, or until the temperature of the media matched that of the water bath. Paddles were then mounted to the Hansen Dissolution Test Station stirring apparatus above the four dissolution vessels such that the distance between the paddle and the die face is 1 inch. The paddle speed is then set to 50 RPM.

### Performing an Intrinsic Dissolution Experiment (Dose Dumping or pH Media):

The pellet prepared as described above is submerged into a vessel prepared as described above, with the pellet surface facing up (metal die up, polypropylene cap facing down). Forceps are used to aid this process so that the pellet apparatus can be gently placed into the bottom of the vessel. A timer is used to track the sampling intervals, and is started when the pellet is dropped into the solution. The lid to the dissolution apparatus is then lowered and the stirring apparatus is activated. Some planning is required in spacing out pellet drops such that each vessel can be sampled at the desired time intervals. Sampling is done by aspirating 5 mL of the solution using a Popper® Micro-Mate® Interchangeable Hypodermic Syringe equipped with a Vortex Pharma Group 10 micron cannula porous filter. This filter should be replaced after each use. Although sampling intervals can change from experiment to experiment, the following has been heavily utilized for the experiments described herein. Sampling occurring at t=1, 3, 5, 10, 15, 30, 45, 60, 90, 120 (in minutes).

### HPLC Methodology

### HPLC Procedure for Analyzing Organic Acid Addition Salts:

All samples should be analyzed with bracketing standard injections of oxymorphone hydrochloride. The standard used should be from a qualified vendor with a known purity, (e.g. oxymorphone hydrochloride, Mallinckrodt). Standard solutions should be prepared to have a concentration that is approximate to that of the samples being analyzed. All samples were run on a Waters Alliance 2695D Separations Module equipped with a Waters 2487 Dual Wavelength Detector detecting at 282 nm. The instrument was equipped with an Agilent 300 Extend-C18 5 μm 4.6×250 mm Zorbax column. The instrument was then plumbed with the proper solutions mentioned above in the section titled “Preparation of Solutions”. The instrument is then set to initial column conditions (see gradient table below):

| Time | % | % |
|---|---|---|
| (minutes) | A | B |
| 0.00 | 90 | 10 |
| 2.00 | 90 | 10 |
| 8.00 | 25 | 75 |
| 8.01 | 0 | 100 |
| 13.00 | 0 | 100 |
| 13.01 | 90 | 10 |
| 17.00 | 90 | 10 |

This method can be used to generate data which can be plotted to provide a dissolution profile of the analyte in question.

The invention has been described with reference to the preferred embodiments without limit thereto. One of skill in the art would realize additional embodiments and improvements which are not specifically set forth herein but which are within the scope of the invention as more specifically set forth in the claims appended hereto.

1. A drug substance consisting essentially of a pharmaceutically acceptable organic acid addition salt of an amine containing pharmaceutically active compound wherein said drug substance is selected from the group consisting of:
amorphous oxymorphone pamoate characterized by at least one method selected from the group consisting of:
a differential scanning calorimeter thermogram of FIG. 1;
an FTIR of FIG. 2;
an X-ray diffraction diffractogram of FIG. 3; and
a ¹H NMR spectrum of FIG. 4;
polymorphic oxymorphone pamoate characterized by at least one method selected from the group consisting of:
a differential scanning calorimeter thermogram of FIG. 5;
an FTIR of FIG. 6;
an X-ray diffraction diffractogram of FIG. 7; and
a ¹H NMR spectrum of FIG. 8;
oxymorphone xinafoate characterized by at least one method selected from the group consisting of:
a differential scanning calorimeter thermogram of FIG. 9
an FTIR of FIG. 10;
an X-ray diffraction diffractogram of FIG. 11; and
a ¹H NMR spectrum of FIG. 12;
amorphous codeine pamoate characterized by at least one method selected from the group consisting of:
a differential scanning calorimeter thermogram of FIG. 13;
an FTIR of FIG. 14;
an X-ray diffraction diffractogram of FIG. 15; and
a ¹H NMR spectrum of FIG. 16;
codeine xinafoate characterized by at least one method selected from the group consisting of:
a differential scanning calorimeter thermogram of FIG. 17;
an FTIR of FIG. 18;
an X-ray diffraction diffractogram of FIG. 19; and
a ¹H NMR spectrum of FIG. 20;
amorphous levorphanol pamoate characterized by at least one method selected from the group consisting of:
a differential scanning calorimeter thermogram of FIG. 21;
an FTIR of FIG. 22;
an X-ray diffraction diffractogram of FIG. 23; and
a ¹H NMR spectrum of FIG. 24;
polymorphic levorphanol pamoate characterized by at least one method selected from the group consisting of:
a differential scanning calorimeter thermogram of FIG. 25;
an FTIR of FIG. 26;
an X-ray diffraction diffractogram of FIG. 27; and
a ¹H NMR spectrum of FIG. 28;
levorphanol xinafoate characterized by at least one method selected from the group consisting of:
a differential scanning calorimeter thermogram of FIG. 29;
an FTIR of FIG. 30;
an X-ray diffraction diffractogram of FIG. 31; and
a ¹H NMR spectrum of FIG. 32;
amorphous naltrexone pamoate characterized by at least one method selected from the group consisting of:
a differential scanning calorimeter thermogram of FIG. 33;
an FTIR of FIG. 34;
an X-ray diffraction diffractogram of FIG. 35; and
a ¹H NMR spectrum of FIG. 36;
polymorphic naltrexone pamoate characterized by at least one method selected from the group consisting of:
a differential scanning calorimeter thermogram of FIG. 37;
an FTIR of FIG. 38;
an X-ray diffraction diffractogram of FIG. 39; and
a ¹H NMR spectrum of FIG. 40; and
naltrexone xinafoate characterized by at least one method selected from the group consisting of:
a differential scanning calorimeter thermogram of FIG. 41;
an FTIR of FIG. 42;
an X-ray diffraction diffractogram of FIG. 43; and
a ¹H NMR spectrum of FIG. 44.

2. The drug substance of claim 1 wherein said amorphous oxymorphone pamoate exhibits an extended release of said oxymorphone from said pamoate in 0.1 N HCl.

3. The drug substance of claim 1 wherein said amorphous oxymorphone pamoate has a rate of release of said oxymorphone from said pamoate in 0.1 N HCl which is not exceeded by a rate of release of said oxymorphone from said pamoate in 0.1 N HCl with 5% ethanol.

4. The drug substance of claim 1 wherein said polymorphic oxymorphone pamoate exhibits immediate release of said oxymorphone from said pamoate in 0.1 N HCl.

5. The drug substance of claim 1 wherein said polymorphic oxymorphone pamoate exhibits extended release of said oxymorphone from said pamoate at pH 4.5, pH 6.8 and in water.

6. The drug substance of claim 1 wherein said oxymorphone xinafoate exhibits extended release of said oxymorphone from said xinafoate in 0.1 N HCl.

7. The drug substance of claim 1 wherein said oxymorphone xinafoate exhibits immediate release of said oxymorphone from said xinafoate at pH 6.8.

8. The drug substance of claim 1 wherein said amorphous codeine pamoate exhibits extended releases of said codeine from said pamoate in 0.1 N HCl.

9. The drug substance of claim 1 wherein said amorphous codeine pamoate has a rate of release of said codeine from said pamoate in 0.1 N HCl which is not exceeded by a rate of release of said codeine from said pamoate in 0.1 N HCl with 5% ethanol.

10. The drug substance of claim 1 wherein said codeine xinafoate exhibits extended release of said codeine from said xinafoate in 0.1 N HCl.

11. The drug substance of claim 1 wherein said codeine xinafoate exhibits immediate release of said codeine from said xinafoate at pH 6.8.

12. The drug substance of claim 1 wherein said amorphous levorphanol pamoate exhibits an extended release of said levorphanol from said pamoate in 0.1 N HCl.

13. The drug substance of claim 1 wherein said amorphous levorphanol pamoate exhibits an extended release of said levorphanol from said pamoate in 0.1 N HCl with 5% ethanol.

14. The drug substance of claim 1 wherein said polymorphic levorphanol pamoate exhibits slow release of said levorphanol from said pamoate in 0.1 N HCl.

15. The drug substance of claim 1 wherein said polymorphic levorphanol pamoate has a rate of release of said levorphanol from said pamoate in 0.1 N HCl which is not exceeded by a rate of release of said levorphanol from said pamoate in 0.1 N HCl with 20% ethanol.

16. The drug substance of claim 1 wherein said levorphanol xinafoate exhibits extended release of said levorphanol from said xinafoate in 0.1 N HCl.

17. The drug substance of claim 1 wherein said levorphanol xinafoate has a rate of release of said levorphanol from said xinafoate in 0.1 N HCl which is not exceeded by a rate of release of said levorphanol from said xinafoate in 0.1 N HCl with 5% ethanol.

18. The drug substance of claim 1 wherein said amorphous naltrexone pamoate exhibits extended release of said naltrexone from said pamoate in 0.1 N HCl.

19. The drug substance of claim 1 wherein said amorphous naltrexone pamoate exhibits extended release of said naltrexone from said pamoate in 0.1 N HCl with ethanol.

20. The drug substance of claim 1 wherein said polymorphic naltrexone pamoate exhibits extended release of said naltrexone from said pamoate in 0.1 N HCl.

21. The drug substance of claim 1 wherein said polymorphic naltrexone pamoate exhibits extended release of said naltrexone from said pamoate at pH 4.5.

22. The drug substance of claim 1 wherein said naltrexone xinafoate exhibits slow release of said naltrexone from said xinafoate in 0.1 N HCl.

23. The drug substance of claim 1 wherein said naltrexone xinafoate exhibits extended release of said naltrexone from said xinafoate at pH 4.5.

24. A drug product comprising at least one drug substance of claim 1.

25. The drug product of claim 24 further comprising an enteric coating.

26. The drug product of claim 24 comprising at least one drug substance selected from the group consisting of said amorphous oxymorphone pamoate, said polymorphic oxymorphone pamoate, said oxymorphone xinafoate, said amorphous codeine pamoate, said codeine xinafoate, said amorphous levorphanol pamoate, said polymorphic levorphanol pamoate and said levorphanol xinafoate and at least one drug substance selected from the group consisting of said amorphous naltrexone pamoate, said polymorphic naltrexone pamoate and said naltrexone xinafoate.

27. The drug product of claim 24 in a form selected from a tablet, a capsule, a caplet, a suspension and an injectable.

28. The drug product of claim 24 further comprising a compound defined by Formula H: 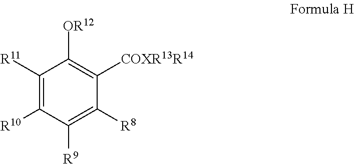
wherein R⁸-R¹¹ are independently selected from H, alkyl or substituted alkyl of 1-6 carbons, adjacent groups may be taken together to form a cyclic alkyl, cyclic alkyl-aryl, or cyclic aryl moiety;
R¹² is selected from H, or an alkali earth cation;
R¹³ and R¹⁴ are independently selected from H, alkyl of 1-6 carbons, an alkali earth cation, and aryl of 6 to 12 carbons, in a number sufficient to complete the valence bonding of X, and
wherein X is selected from nitrogen, oxygen or sulfur.

29. The drug product of claim 28 wherein said Formula H is selected from 3-hydroxy-2-naphthoic acid (BON Acid), disodium pamoate, and pamoic acid.

30. An oral dose drug product exhibiting preferential release in the bowel wherein said drug product comprises a drug substance selected from the group consisting of oxymorphone xinafoate and codeine xinafoate.

31. The drug product of claim 30 wherein the said preferential release occurs at a pH above 4.5.

32. The drug product of claim 30 wherein said drug product does not comprise an enteric coating.

33. The drug product of claim 30 wherein said oxymorphone xinafoate is characterized by at least one method selected from the group consisting of:
a differential scanning calorimeter thermogram of FIG. 9
an FTIR of FIG. 10;
an X-ray diffraction diffractogram of FIG. 11; and
a ¹H NMR spectrum of FIG. 12.

34. The drug product of claim 30 wherein said oxymorphone xinafoate exhibits essentially no release of said oxymorphone from said xinafoate in 0.1 N HCl.

35. The drug product of claim 30 wherein said oxymorphone xinafoate exhibits immediate release of said oxymorphone from said xinafoate at pH 6.8.

36. The drug product of claim 30 wherein said codeine xinafoate is characterized by at least one method selected from the group consisting of:
a differential scanning calorimeter thermogram of FIG. 17;
an FTIR of FIG. 18;
an X-ray diffraction diffractogram of FIG. 19; and
a ¹H NMR spectrum of FIG. 20.

37. The drug product of claim 30 wherein said codeine xinafoate exhibits extended release of said codeine from said xinafoate in 0.1 N HCl.

38. The drug product of claim 30 wherein said codeine xinafoate exhibits immediate release of said codeine from said xinafoate at pH 6.8.

39. The drug product of claim 30 further comprising a compound defined by Formula H: 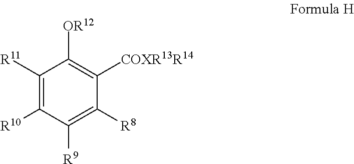
wherein R⁸-R¹¹ are independently selected from H, alkyl or substituted alkyl of 1-6 carbons, adjacent groups may be taken together to form a cyclic alkyl, cyclic alkyl-aryl, or cyclic aryl moiety;
R¹² is selected from H, or an alkali earth cation;
R¹³ and R¹⁴ are independently selected from H, alkyl of 1-6 carbons, an alkali earth cation, and aryl of 6 to 12 carbons, in a number sufficient to complete the valence bonding of X, and
wherein X is selected from nitrogen, oxygen or sulfur.

40. The drug product of claim 39 wherein said Formula H is selected from 3-hydroxy-2-naphthoic acid (BON Acid), disodium pamoate, and pamoic acid.

41. A solid oral dose drug product comprising at least one drug substance selected from the group consisting of:
amorphous oxymorphone pamoate characterized by:
an extended release of said oxymorphone from said pamoate in 0.1 N HCl; or
a rate of release of said oxymorphone from said pamoate in 0.1 N HCl which is not exceeded by a rate of release of said oxymorphone from said pamoate in 0.1 N HCl with 5% ethanol;
polymorphic oxymorphone pamoate characterized by at one of:
immediate release of said oxymorphone from said pamoate in 0.1 N HCl; or
extended release of said oxymorphone from said pamoate at pH 4.5;
oxymorphone xinafoate characterized by at least one of:
extended release of said oxymorphone from said xinafoate in water; or
immediate release of said oxymorphone from said xinafoate at pH 6.8;
amorphous codeine pamoate characterized by at least one of:
extended releases of said codeine from said pamoate in 0.1 N HCl; or
a rate of release of said codeine from said pamoate in 0.1 N HCl which is not exceeded by a rate of release of said codeine from said pamoate in 0.1 N HCl with 5% ethanol;
codeine xinafoate characterized by at least one of:
extended release of said codeine from said xinafoate in water; or
immediate release of said codeine from said xinafoate at pH 6.8;
amorphous levorphanol pamoate characterized by at least one of:
extended release of said levorphanol from said pamoate in 0.1 N HCl; or
extended release of said levorphanol from said pamoate in 0.1 N HCl with 5% ethanol;
polymorphic levorphanol pamoate characterized by at least one of:
extended release of said levorphanol from said pamoate in 0.1 N HCl; or
a rate of release of said levorphanol from said pamoate in 0.1 N HCl which is not exceeded by a rate of release of said levorphanol from said pamoate in 0.1 N HCl with 20% ethanol;
levorphanol xinafoate characterized by at least one of:
extended release of said levorphanol from said xinafoate in 0.1 N HCl; or
a rate of release of said levorphanol from said xinafoate in 0.1 N HCl which is not exceeded by a rate of release of said levorphanol from said xinafoate in 0.1 N HCl with 5% ethanol;
amorphous naltrexone pamoate characterized by at least one of:
extended release of said naltrexone from said pamoate in 0.1 N HCl; or
extended release of said naltrexone from said pamoate in 0.1 N HCl with ethanol;
polymorphic naltrexone pamoate characterized by at least one of:
extended release of said naltrexone from said pamoate in 0.1 N HCl; or
extended release of said naltrexone from said pamoate at pH 4.5; and
naltrexone xinafoate characterized by at least one of:
extended release of said naltrexone from said xinafoate in 0.1 N HCl; or
a rate of release of said naltrexone from said xinafoate in 0.1 N HCl which is not exceeded by a rate of release of said naltrexone from said xinafoate in 0.1 N HCl with 5% ethanol.

42. The solid oral dose drug product of claim 41 wherein said amorphous oxymorphone pamoate exhibits an extended release of said oxymorphone from said pamoate in 0.1 N HCl.

43. The solid oral dose drug product of claim 41 wherein said amorphous oxymorphone pamoate has a rate of release of said oxymorphone from said pamoate in 0.1 N HCl which is not exceeded by a rate of release of said oxymorphone from said pamoate in 0.1 N HCl with 5% ethanol.

44. The solid oral dose drug product of claim 41 wherein said polymorphic oxymorphone pamoate exhibits immediate release of said oxymorphone from said pamoate in 0.1 N HCl.

45. The solid oral dose drug product of claim 41 wherein said polymorphic oxymorphone pamoate exhibits extended release of said oxymorphone from said pamoate at pH 4.5.

46. The solid oral dose drug product of claim 41 wherein said oxymorphone xinafoate exhibits extended release of said oxymorphone from said xinafoate in 0.1 N HCl.

47. The solid oral dose drug product of claim 41 wherein said oxymorphone xinafoate exhibits immediate release of said oxymorphone from said xinafoate at pH 6.8.

48. The solid oral dose drug product of claim 41 wherein said amorphous codeine pamoate exhibits extended releases of said codeine from said pamoate in 0.1 N HCl.

49. The solid oral dose drug product of claim 41 wherein said amorphous codeine pamoate has a rate of release of said codeine from said pamoate in 0.1 N HCl which is not exceeded by a rate of release of said codeine from said pamoate in 0.1 N HCl with 5% ethanol.

50. The solid oral dose drug product of claim 41 wherein said codeine xinafoate exhibits extended release of said codeine from said xinafoate in 0.1 N HCl.

51. The solid oral dose drug product of claim 41 wherein said codeine xinafoate exhibits immediate release of said codeine from said xinafoate at pH 6.8.

52. The solid oral dose drug product of claim 41 wherein said amorphous levorphanol pamoate exhibits extended release of said levorphanol from said pamoate in 0.1 N HCl.

53. The solid oral dose drug product of claim 4 wherein said amorphous levorphanol pamoate exhibits an extended release of said levorphanol from said pamoate in 0.1 N HCl with 5% ethanol.

54. The solid oral dose drug product of claim 41 wherein said polymorphic levorphanol pamoate exhibits extended release of said levorphanol from said pamoate in 0.1 N HCl.

55. The solid oral dose drug product of claim 41 wherein said polymorphic levorphanol pamoate has a rate of release of said levorphanol from said pamoate in 0.1 N HCl which is not exceeded by a rate of release of said levorphanol from said pamoate in 0.1 N HCl with 20% ethanol.

56. The solid oral dose drug product of claim 41 wherein said levorphanol xinafoate exhibits extended release of said levorphanol from said xinafoate in 0.1 N HCl.

57. The solid oral dose drug product of claim 41 wherein said levorphanol xinafoate has a rate of release of said levorphanol from said xinafoate in 0.1 N HCl which is not exceeded by a rate of release of said levorphanol from said xinafoate in 0.1 N HCl with 5% ethanol.

58. The solid oral dose drug product of claim 41 wherein said amorphous naltrexone pamoate exhibits extended release of said naltrexone from said pamoate in 0.1 N HCl.

59. The solid oral dose drug product of claim 41 wherein said amorphous naltrexone pamoate exhibits extended release of said naltrexone from said pamoate in 0.1 N HCl with ethanol.

60. The solid oral dose drug product of claim 41 wherein said polymorphic naltrexone pamoate exhibits extended release of said naltrexone from said pamoate in 0.1 N HCl.

61. The solid oral dose drug product of claim 41 wherein said polymorphic naltrexone pamoate exhibits extended release of said naltrexone from said pamoate at pH 4.5.

62. The solid oral dose drug product of claim 41 wherein said naltrexone xinafoate exhibits extended release of said naltrexone from said xinafoate in 0.1 N HCl.

63. The solid oral dose drug product of claim 41 wherein said naltrexone xinafoate exhibits extended release of said naltrexone from said xinafoate at pH 4.5.

64. The solid oral dose drug product of claim 41 further comprising an enteric coating.

65. The solid oral dose drug product of claim 41 which does not comprise an enteric coating.

66. The solid oral dose drug product of claim 41 further comprising a compound defined by Structure H: 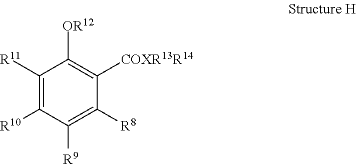
wherein R⁸-R¹¹ are independently selected from H, alkyl or substituted alkyl of 1-6 carbons, adjacent groups may be taken together to form a cyclic alkyl, cyclic alkyl-aryl, or cyclic aryl moiety;
R¹² is selected from H, or an alkali earth cation;
R¹³ and R¹⁴ are independently selected from H, alkyl of 1-6 carbons, an alkali earth cation, and aryl of 6 to 12 carbons, in a number sufficient to complete the valence bonding of X, and
wherein X is selected from nitrogen, oxygen or sulfur.

67. The solid oral dose drug product of claim 66 wherein said Formula H is selected from 3-hydroxy-2-naphthoic acid (BON Acid), disodium pamoate, and pamoic acid.

68. A drug product comprising:
a drug substance consisting of an organic acid addition salt of naltrexone wherein said organic acid addition salt is defined by Structure A: 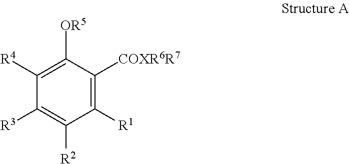
wherein:
R¹-R⁴ are independently selected from H, alkyl or substituted alkyl of 1-6 carbons, adjacent groups may be taken together to form a cyclic alkyl or cyclic aryl moiety;
R⁵ represents H, alkyl, alkylacyl or arylacyl;
R⁶ and R⁷ are independently selected from H, alkyl of 1-6 carbons, aryl of 6-12 carbons, alkylacyl or arylacyl analogues sufficient to satisfy the valence of X;
X is selected from nitrogen, oxygen or sulfur, and when X═O, R⁶+R⁷ may represent an alkali earth cation, ammonium or together form a heterocyclic moiety.

69. The drug product of claim 68 wherein said drug substance is selected from the group consisting of amorphous naltrexone pamoate, polymorphic naltrexone pamoate and naltrexone xinafoate.

70. The drug product of claim 69 wherein said amorphous naltrexone pamoate is characterized by at least one method selected from the group consisting of:
a differential scanning calorimeter thermogram of FIG. 33;
an FTIR of FIG. 34;
an X-ray diffraction diffractogram of FIG. 35; and
a ¹H NMR spectrum of FIG. 36.

71. The drug product of claim 69 wherein said polymorphic naltrexone pamoate is characterized by at least one method selected from the group consisting of:
a differential scanning calorimeter thermogram of FIG. 37;
an FTIR of FIG. 38;
an X-ray diffraction diffractogram of FIG. 39; and
a ¹H NMR spectrum of FIG. 40.

72. The drug product of claim 69 wherein said naltrexone xinafoate is characterized by at least one method selected from the group consisting of:
a differential scanning calorimeter thermogram of FIG. 41;
an FTIR of FIG. 42;
an X-ray diffraction diffractogram of FIG. 43; and
a ¹H NMR spectrum of FIG. 44.

73. The drug product of claim 69 wherein said amorphous naltrexone pamoate is characterized by at least one of:
extended release of said naltrexone from said pamoate in 0.1 N HCl; or
extended release of said naltrexone from said pamoate in 0.1 N HCl with ethanol.

74. The drug product of claim 69 wherein said polymorphic naltrexone pamoate is characterized by at least one of:
extended release of said naltrexone from said pamoate in 0.1 N HCl; or
extended release of said naltrexone from said pamoate at pH 4.5.

75. The drug product of claim 69 wherein said naltrexone xinafoate is characterized by at least one of:
extended release of said naltrexone from said xinafoate in 0.1 N HCl; or
extended release of said naltrexone from said xinafoate at pH 4.5.

76. The drug product of claim 68 further comprising an opioid.

77. The drug product of claim 76 wherein said opioid is selected from the group consisting of codeine, hydrocodone, propoxyphene, fentanyl, hydromorphone, levorphanol, meperidine, methadone, morphine, oxycodone, oxymorphone, buprenorphine, butorphanol, nalbuphine, and pentazocine.

78. The drug product of claim 77 wherein said opioid is selected from the group consisting of oxymorphone, codeine and levorphanol.

79. The drug product of claim 76 wherein said opioid is selected from the group consisting of oxymorphone pamoate, oxymorphone xinafoate, codeine pamoate, codeine xinafoate, levorphanol pamoate, and levorphanol xinafoate.

80. A method of administering an active pharmaceutical comprising:
providing a drug substance selected from the group consisting of amorphous oxymorphone pamoate, amorphous codeine pamoate, polymorphic levorphanol pamoate and levorphanol xinafoate;
forming a drug product comprising said drug substance suitable for achieving a therapeutic dose of said drug substance in a predetermined time; and
wherein when administered said therapeutic dose is not exceeded in said predetermined time by ingestion of alcohol at biological pH.

81. The method of administering an active pharmaceutical of claim 80 wherein said amorphous oxymorphone pamoate characterized by at least one method selected from the group consisting of:
a differential scanning calorimeter thermogram of FIG. 1;
an FTIR of FIG. 2;
an X-ray diffraction diffractogram of FIG. 3; and
a ¹H NMR spectrum of FIG. 4.

82. The method of administering an active pharmaceutical of claim 80 wherein said amorphous codeine pamoate characterized by at least one method selected from the group consisting of:
a differential scanning calorimeter thermogram of FIG. 13;
an FTIR of FIG. 14;
an X-ray diffraction diffractogram of FIG. 15; and
a ¹H NMR spectrum of FIG. 16.

83. The method of administering an active pharmaceutical of claim 80 wherein said polymorphic levorphanol pamoate is characterized by at least one method selected from the group consisting of:
a differential scanning calorimeter thermogram of FIG. 25;
an FTIR of FIG. 26;
an X-ray diffraction diffractogram of FIG. 27; and
a ¹H NMR spectrum of FIG. 28.

84. The method of administering an active pharmaceutical of claim 80 wherein said levorphanol xinafoate is characterized by at least one method selected from the group consisting of:
a differential scanning calorimeter thermogram of FIG. 29;
an FTIR of FIG. 30;
an X-ray diffraction diffractogram of FIG. 31; and
a ¹H NMR spectrum of FIG. 32.

85. The method of administering an active pharmaceutical of claim 80 wherein said amorphous oxymorphone pamoate exhibits an extended release of said oxymorphone from said pamoate in 0.1 N HCl.

86. The method of administering an active pharmaceutical of claim 80 wherein said amorphous oxymorphone pamoate has a rate of release of said oxymorphone from said pamoate in 0.1 N HCl which is not exceeded by a rate of release of said oxymorphone from said pamoate in 0.1 N HCl with 5% ethanol.

87. The method of administering an active pharmaceutical of claim 80 wherein said amorphous codeine pamoate exhibits extended release of said codeine from said pamoate in 0.1 N HCl.

88. The method of administering an active pharmaceutical of claim 80 wherein said amorphous codeine pamoate has a rate of release of said codeine from said pamoate in 0.1 N HCl which is not exceeded by a rate of release of said codeine from said pamoate in 0.1 N HCl with 5% ethanol.

89. The method of administering an active pharmaceutical of claim 80 wherein said polymorphic levorphanol pamoate exhibits extended release of said levorphanol from said pamoate in 0.1 N HCl.

90. The method of administering an active pharmaceutical of claim 80 wherein said polymorphic levorphanol pamoate has a rate of release of said levorphanol from said pamoate in 0.1 N HCl which is not exceeded by a rate of release of said levorphanol from said pamoate in 0.1 N HCl with 20% ethanol.

91. The method of administering an active pharmaceutical of claim 80 wherein said levorphanol xinafoate exhibits extended release of said levorphanol from said xinafoate in 0.1 N HCl.

92. The method of administering an active pharmaceutical of claim 80 wherein said levorphanol xinafoate has a rate of release of said levorphanol from said xinafoate in 0.1 N HCl which is not exceeded by a rate of release of said levorphanol from said xinafoate in 0.1 N HCl with 5% ethanol.

93. A solid oral dose drug product comprising a mixture of polymorphic oxymorphone pamoate and oxymorphone xinafoate drug substances providing an immediate release therapeutic dosage of said oxymorphone from said pamoate within 30 minutes under gastric conditions and providing extended release of said oxymorphone from said oxymorphone xinafoate.

94. A solid oral dose drug product according to claim 93 providing an effective therapeutic dosage of oxymorphone to a patient in need of said oxymorphone for a period of from about thirty minutes after ingestion to about twenty-four hours after ingestion.

95. A solid oral dose drug product according to claim 93 comprising a third drug substance selected from the group of naltrexone hydrochloride, naltrexone pamoate and naltrexone xinafoate.

96. A solid oral dose dug product comprising a mixture of drug substances selected from the group consisting of codeine sulfate, codeine pamoate and codeine xinafoate providing immediate release therapeutic dosage of said codeine released from said codeine sulfate under gastric conditions, a pulsed dosage release corresponding to release of said codeine from said codeine xinafoate at a point from thirty minutes to three hours after ingestion, and an extended release of codeine from said codeine pamoate for patient treatment up to twenty-four hours after said drug product ingestion.

97. A solid oral dose drug product according to claim 96 wherein said drug substances are selected from codeine sulfate and codeine pamoate.

98. A solid oral dose drug product according to claim 96 comprising a fourth drug substance selected from the group of naltrexone hydrochloride, naltrexone pamoate and naltrexone xinafoate.

99. A solid oral dose drug product comprising a mixture of drug substances selected from levorphanol tartrate, levorphanol pamoate, and levorphanol xinafoate providing an immediate release therapeutic dosage of said levorphanol from said levorphanol tartrate under gastric conditions and an extended release of said levorphanol from levorphanol pamoate or xinafoate, said extended release providing therapeutic dosage up to twenty-four hours after ingestion by the patient.

100. A solid oral dose drug product according to claim 99 and further comprising a drug substance selected from the group of naltrexone hydrochloride, naltrexone pamoate and naltrexone xinafoate.
